(19) United States
(12) Patent Application Publication
(10) Pub. No.: US 2008/0275058 A1
(43) Pub. Date: Nov. 6, 2008

(54) N-Substituted-1H-Quinoline-2,4-Diones, Preparation Method Thereof, And Pharmaceutical Composition Containing The Same

(75) Inventors: Churlmin Seong, Seoul (KR); Nosang Park, Yuseong-gu Daejeon (KR); Jinil Choi, Yuseong-gu Daejeon (KR); Wookyu Park, Chungcheongbuk-do (KR); Jaeyang Kong, Daejeon (KR); Chulmin Park, Yuseong-gu Daejeon (KR)

Correspondence Address: LUCAS & MERCANTI, LLP, 475 PARK AVENUE SOUTH, 15TH FLOOR, NEW YORK, NY, 10016, US

(73) Assignee: KOREA RESEARCH INSTITUTE OF CHEMICAL TECHNOLOGY, Daejeon (KR)

(21) Appl. No.: 12/065,565

(22) PCT Filed: Nov. 23, 2005
(86) PCT No.: PCT/KR05/03969

§ 371 Date: Mar. 3, 2008

(30) Foreign Application Priority Data
Sep. 15, 2005 (KR) 10-2005-0086361
Nov. 22, 2005 (KR) 10-2005-0111634

(51) Int. Cl. A61K 31/496; C07D 401/04; A61P 25/28; A61P 25/24

(52) U.S. Cl. 514/253.07; 544/363

(57) ABSTRACT

The present invention relates to compounds of N-substituted -1H-quinoline-2,4-diones acting as a 5HT6 receptor antagonist, a preparation method thereof, and a pharmaceutical composition containing the same for treatment of the central nervous system disorders. The compounds of N-substituted-1H-quinoline-2,4-diones according to the present invention have excellent binding affinity for the 5HT6 receptor and excellent selectivity for the 5HT6 receptor over other receptors. Also, the compounds reverse a disruption of PPI by methamphetamine and don't show rotatod deficit in mice. Thereof the compounds according to the present invention may be valuably used for treatment if a 5HT6 receptor relating disorder

### TECHNICAL FIELD

The present invention relates to N-substituted -1H-quinoline-2,4-diones acting as a 5-HT6 receptor antagonist, a preparation method thereof, and a pharmaceutical composition containing the same for treatment of central nervous system disorders.

### BACKGROUND ART

Although the function of serotonin (5-HT) in the central nervous system is still being clarified, various studies have indicated 5-HT has been implicated in the aetiology of many disease states and may be particularly important in mental illness, such as depression, anxiety, schizophrenia, eating disorders, obsessive compulsive disorder (OCD), migraine and panic disorder. Recent advances in pharmacology, molecular biology, and genetics on the serotonin system hold out the promise of the development of improved pharmacological treatment for some aspects of neurological diseases. Indeed, many currently used treatments of these disorders are thought to act by modulating serotonergic neurons. During the last decade, multiple 5-HT receptor subtypes have been characterized. Initially, receptor subtypes were characterized using pharmacological tools only. On the basis of the receptor binding profiles, common secondary messenger coupling and the functional activity of ligands, four main subgroups of 5-HT receptors, termed 5-HT1, 5-HT2, 5-HT3 and 5-HT4, were identified. More recently, molecular biological techniques have both confirmed this classification, in that each subgroup has been found to have relatively dissimilar protein structures, and led to the identification of novel 5-HT receptors (5-HT1F, 5-HT5, 5-HT6 and 5-HT7) enabling them to be cloned, expressed in cultured cell lines [Hoyer, D. et al., Pharmacol. Biochem. Behav., 2002, 71, 533-554; Kroeze, W. K. et al., Curr. Top. Med. Chem., 2002, 2, 507-528].

Most recently, the 5-HT6 receptor has been cloned from rat cDNA based on its homology to previously cloned G-protein-coupled receptors. The rat receptor consists of 438 amino acids with seven transmembrane domains and is positively coupled to adenylyl cyclase via the Gs G-protein [Monsma, F. J. et al., Mol. Pharmacol., 1993, 43, 320-327]. Human 5-HT6 receptors, a 440 amino acid polypeptide, display 89% overall sequence homology with the rat receptors and is positively coupled to an adenylate cyclase second messenger system [Kohen, R. et al., J. Neurochem., 1996, 66, 47-56]. Rat and human 5-HT6 mRNA is located in the striatum, amygdala, nucleus accumbens, hippocampus, cortex and olfactory tubercle, but has not been found in peripheral organs studied. In pharmacological studies, tritiated 5-HT, tritiated LSD, and [1251]-2-iodo LSD have been used to radiolabel 5-HT6 receptors. 5-HT binds with moderately high affinity(Ki=50-150 nM). Tricyclic antipsychotic agents and some antidepressants bind with significant affinity. A related investigation examined antipsychotics in greater detail and found that representative members of several classes of antipsychotics bind with high affinity. Examples include phenothiazine chlorpromazine, thioxanthene chlorprothixene, diphenylbutylpiperidine pimozide, heterocyclic antipsychotic agent loxapine and clozapine [Roth, B. L. et al., J. Pharmacol. Exp. Ther., 1994, 268, 1403-1410]. These results led to suggestions that 5-HT6 receptors might play a role in certain types of psychoses and that they might represent significant targets for the atypical antipsychotics in particular.

Until selective ligands were developed, exploration of 5-HT6 pharmacology was largely dependent on the use of nonselective agents. In the absence of selective ligands for the receptor, functional studies have been carried out using an antisense approach. 5-HT6 specific antisense produced a specific behavioural syndrome of yawning, stretching and chewing, but had no other discernable action in rats. The non-selective ligands were useful for investigating the pharmacology of 5-HT6 systems in preparations where other 5-HT receptors were absent (e.g., cAMP assays); however, owing to their lack of selectivity, they were of limited value for most other pharmacological studies.

Recent advent of selective agents has greatly benefited 5-HT6 studies, and this field of research has recently exploded. The development of more selective ligands may therefore lead to treatments with increased efficacy and reduced side effects. Alternatively, selective ligands may form completely novel therapies. It was not until 1998 that the first 5-HT6-selective antagonist was described, and this prompted others to quickly report their efforts in this area. Sleight et al. at Hoffman-La Roche Co. identified the bisaryl sulfonamides Ro 04-6790 (1, Ki=55 nM), Ro 63-0563 (2, Ki=12 nM) as very selective 5-HT6 antagonists [Sleight, A. J. et al., Br. J. Pharmacol., 1998, 124, 556-562]. Shortly thereafter, MS-245 (3, Ki=2.3 nM) was reported. Interestingly, although they represented independent discoveries, all three were identified by random screening methods and all three possess a sulfonamide moiety.

One problem associated with these antagonists was their low penetration of the CNS. At the time, Smith-Kline Beecham Co. also pinched out compound 4 via high-throughput screening. It displayed high affinity (Ki=5 nM) for 5-HT6 receptors and >50-fold selectivity over 10 other 5-HT receptors and no measurable affinity for 50 other receptor/binding sites. It was a pure antagonist of cAMP accumulation (pKb=7.8) [Bromidge, S. M. et al., J. Med. Chem., 1999, 42, 202-205]. It was moderately brain penetrant (25%) but subject to rapid blood clearance resulting in low bioavailability.

An ensuing structure activity study identified SB-271046 (5, Ki=1 nM; >200 selectivity over 50 other receptors) retained antagonist activity, and although less brainpenetrant (10%), it showed excellent (>80%) oral bioavailability.

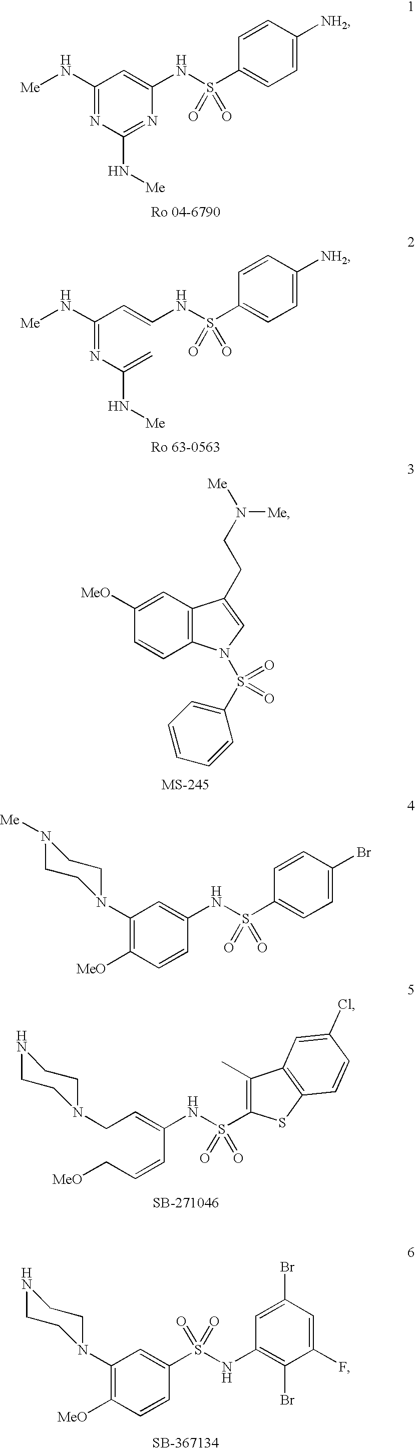

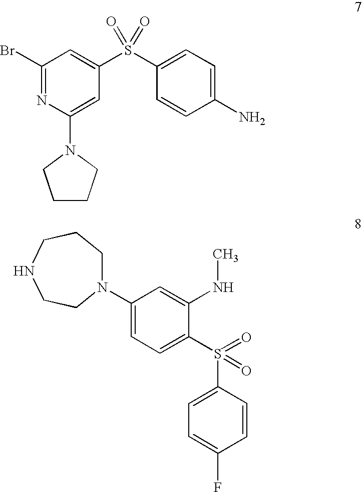

Subsequent studies by this group showed that SB-357134 (6, Ki=3 nM) with a low clearance rate and excellent oral bioavailability. In 1999, Glennon et al. undertook a structure affinity investigation of the binding of tryptamine derivatives at human 5-HT6 receptors [Glennon, R. A. et al., J. Med. Chem., 2000, 43, 1011-10181. MS-245 was found as an antagonist (pA2=8.88) with high affinity (Ki=2.3 nM). In contrast to the above-mentioned sulfonamides or tryptamine derivatives, Hoffmann-LaRoche (7) and Pharmacia-Upjohn (8, Ki=1.4 nM) recently revealed several sulfones [Slassi, A. et al., Expert Opin. Ther. Pat., 2002, 12, 513-527]. Newer agents continue to be developed in attempts to improve pharmacokinetic and pharmacodynamic properties. Now that some tools are available, attention is focusing more and more on the function of 5-HT6 receptors.

A typical antipsychotics, in particular, display high affinity at these receptors (vide supra). In addition, the tritiated atypical antipsychotic agent [3H]clozapine was shown to label two populations of receptors in rat brain and one population was thought to represent 5-HT6 receptors [Glatt, C. E. et al., Mol. Med., 1995, 1, 398-406]. Vogt et al. performed a systematic mutation scan of the coding region of the 5-HT6 receptor gene of 137 individuals (including schizophrenic and depressed patients) and concluded that the gene might be involved in bipolar affective disorder [Vogt, I. R. et al., Am. J. Med. Genet., 2000, 96, 217-221].

Prior to the identification of 5-HT6-selective agents, Bourson et al. demonstrated that intracerebroventricular administration of antisense oligonuceotides produced in rats a specific behavior of yawning, stretching, and chewing, which could be antagonized by atropine [Bourson, A. et al., J. Pharmacol. Exp. Ther., 1995, 274, 173-180]. Sleight et al. demonstrated that Ro 04-6790 (1) was capable of inducing this same effect. Owing to a relationship between cholinergic function and cognition, this led to speculation that 5-HT6 receptors might be involved in memory and cognitive dysfunction [Sleight, A. J. et al., Neuropharmacology, 2001, 41, 210-219; Rogers, D. C. et al., Psychopharmacology (Berlin), 2001, 158, 114-119].

In addition, because antisense oligonucleotide pretreatment and Ro 04-6790 administration both led to decreased food intake by rats, it was suggested that 5-HT6 receptors might be involved in the regulation of feeding. Furthermore, Russell and Dias have questioned the postulate that 5-HT6 antagonists increase cholinergic transmission [Russell, M. G. N.; Dias, R., Curr. Top. Med. Chem., 2002, 2, 643-654].

Despite the mechanistic disagreement, there is evidence for the involvement of 5-HT6 receptors in learning and memory. When a water maze was used with rats as subjects, SB-271046 (5) and SB-357134 (6) showed significant improvement in retention of a previously learned task. Furthermore, SB-271046 (5) increased extracellular glutamate levels in frontal cortex and dorsal hippocampus by several fold, leading to the conclusion that selective enhancement of excitatory neurotransmission by SB-271046 supports a role for 5-HT6 receptor antagonists in the treatment of cognitive disorders and memory dysfunction [Dawson, L. A. et al., Neuropsychopharmacology, 2001, 25, 662-668].

In addition, SB-357134 (6) produced a potent and dose-dependent increase in seizure threshold (rat maximal electroseizure threshold) following oral administration, suggesting possible therapeutic utility in convulsive disorders [Stean, T. O. et al., Pharmacol. Biochem. Behav., 2002, 71, 645-654]. These findings are consistent with an earlier finding that SB-271046 (5) and Ro 04-6790 (1) possess anticonvulsant activity.

Overall, there is some evidence to suggest that 5-HT6 receptors could be involved in psychosis. There is still more evidence that these receptors are involved in cognition and learning and additional evidence that they might have a role in convulsive disorders and appetite control. Although additional studies are certainly warranted, particularly with some of the newer 5-HT6 antagonists that are more brain-penetrant than the earlier agents, the future of 5-HT6 receptor ligands as potential therapeutic agents is quite exciting.

The inventors made an effort to develop a 5-HT6 antagonist having excellent binding affinity and selectivity, and has completed the present invention by discovering that quinoline-2,4-dione derivatives are 5-HT6 antagonists having very excellent binding strength and selectivity compared to sulfonamide or sulfonic structures disclosed in the prior art.

### DISCLOSURE OF INVENTION

### Technical Problem

The present invention provides N-substituted-1H-quinoline-2,4-diones and a pharmaceutically acceptable salt thereof.

Additionally, the present invention provides a preparation method for N-substituted-1H-quinoline-2,4-diones.

Additionally, the present invention provides a pharmaceutical composition including N-substituted-1H-quinoline-2,4-diones, a pharmaceutically acceptable salt thereof or prodrug thereof for treatment of the central nervous system disorders.

### Advantageous Effects

The compounds of N-substituted-1H-quinoline-2,4-diones according to the present invention have excellent binding affinity to the 5-HT6 receptor, excellent selectivity to the 5-HT6 receptor over other receptors, inhibition of the serotonin(5-HT)-stimulated cAMP accumulation and an effect on methamphetamine(2 [symbol] i.p.)-induced disruption of prepulse inhibition (PPI) in rats. Also, the compounds of the present invention below 400 [symbol] don't show any rotarod deficits in mice.

### BRIEF DESCRIPTION OF THE DRAWINGS

FIG. 1 is a graph showing an inhibitory effect of compounds according to the example of the present invention and methiothepin on cAMP accumulation mediated by 5-HT6 receptor of human HeLa cell.

FIG. 2 and FIG. 3 are a graph showing an inhibitory effect of compounds according to the example of the present invention (50 [symbol] i.p.) on hyperactivity of a rat induced by methamphetamine (2 [symbol] i.p.).

### BEST MODE FOR CARRYING OUT THE INVENTION

The present invention provides N-substituted-1H-quinoline-2,4-diones represented by Formula 1, a pharmaceutically acceptable salt and prodrug thereof.

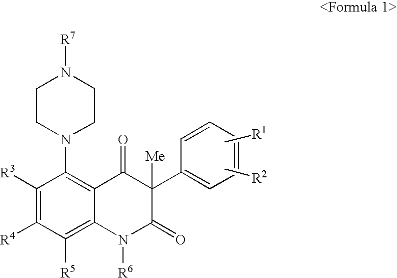

wherein,

R¹ and R²independently represent a hydrogen, halogen, nitro, amino, amino substituted by one or two alkyl, cyclic amino, carboxylic acid, thiol, cyano, alkyl, aryl, heteroaryl, alkoxy, aryloxy, acyloxy, acylamino, arylsulfonylamino, arylsulfonylureido, alkylthio, arylthio, alkylcarboxylate, arylcarboxylate, aralkylcarboxylate, alkylureido, arylureido, alkylamidino or arylamidino.

R³, R⁴ and R⁵ independently represent a hydrogen, halogen, amino, cyclic amino, nitro, cyano, alkyl, haloalkyl, alkoxy, haloalkoxy, piperidinyl or N-methyl piperidinyl.

R⁶ represents alkyl, aryl, cycloalkyl, arylalkyl, heteroaryl or heteroarylalkyl.

R⁷ represents hydrogen, alkyl or aryl.

Term “alkyl” as used herein means straight and branched chain containing from 1 to 7 carbon atoms, and includes methyl, ethyl, propyl, isopropyl, butyl, sec-butyl, and tert-butyl, pentyl, hexyl, cyclopropylmethyl, cyclohexylmethyl group and the like.

The term “cycloalkyl” refers to carbocyclic ring containing from 3 to 7 carbon atoms, and includes cyclopropyl, cyclobutyl, cyclopentyl, cyclohexyl, cycloheptyl group and the like.

Term “alkoxy” as used herein means straight and branched alkoxy groups containing from 1 to 7 carbon atoms, and includes methoxy, ethoxy, propyloxy, iso-propyloxy, butoxy, sec-butoxy, and tert-butoxy, pentoxy, hexyloxy, cyclo-hexylmethoxy group and the like.

Term “haloalkyl” means alkyl groups substituted by one or more fluorine, chlorine, e.g. fluoromethyl, difluoromethyl, trifluoromethyl, pentafluoroethyl, 1,1-difluoroethyl and trichloromethyl group.

The term “aryl” refers to carbocylic aromatic group, includes phenyl, naphthyl, phenanthryl, anthracyl, indenyl, biphenyl, fluorenyl group and the like.

The term “heteroaryl” refers to an aryl group containing from 1 to 3 selected from O, N and S, and includes pyridyl, quinolinyl, isoquinolinyl, pyridazinyl, pyrimidinyl, pyrazinyl, pyrrolyl, indolyl, pyranyl, furyl, benzimidazolyl, benzofuryl, thienyl, benzthienyl, imidazolyl, oxadiazolyl, thiazolyl, thiadiazolyl group and the like.

The “aryl” and “heteroaryl” groups are optionally substituted by 1, 2 or 3 independently selected substituents which include alkyl, alkoxy, halogen, nitro, amino, cyano, hydroxy and cyclic amino group.

The term “heteroarylalkyl” refers to alkyl groups containing above-mentioned heteroaryl groups. As the same way, the term “arylalkyl” refers to alkyl groups containing above-mentioned aryl groups.

The term “amino”includes NH₂, NHR₅ and NR₅R₆, wherein R₅ and R₆ are C₁˜C₄ alkyl group. The term “cyclic amino” includes piperidyl, piperazinyl and morpholinyl group.

Typically, the halogen includes fluorine, chlorine, bromine and iodine.

Preferably,

R¹ and R² are independently a hydrogen, halogen, C₁˜C₄ alkoxy, amino, amino substituted by one or two C₁˜C₄ alkyl, nitro or benzyloxy;

R³, R⁴ and R⁵ are independently a hydrogen, halogen or C₁˜C₄ alkoxy;

R⁶ represents a C₁˜C₄ alkyl; C₃˜C₇ cycloalkyl C₁˜C₂ alkyl; benzyl substituted by a substituent selected from a group comprising of hydrogen, nitro, amino, halogen and C₁˜C₄ alkoxyphenyl; naphthalenylmethyl; or heteroaryl C₁˜C₂ alkyl substituted by a substituent selected from a group comprising of pirydine, quinoline and benzoimidazole; and

R⁷is a hydrogen or C₁˜C₄ alkyl.

More preferably,

R¹ is a hydrogen, fluorine, chlorine, bromine, iodine, methoxy, ethoxy, amino, methylamino, ethylamino, dimethylamino, diethyamino, nitro or benzyloxy.

R²is a hydrogen, fluorine, chlorine, bromine, iodine, methoxy, nitro, amino or benzyloxy.

R³, R⁴ and R⁵ are independently a hydrogen, chlorine, bromine or methoxy.

R⁶represents a methyl, ethyl, cyclohexylmethyl, benzyl, nitrobenzyl, aminobenzyl, methoxybenzyl, bromobenzyl, biphenylmethyl, naphthalenylmethyl, pyridinylmethyl, quinolinylmethyl or benzoimidazolylmethyl.

R⁷ is a hydrogen, methyl or ethyl.

Salts of the compounds of Formula 1 according to the present invention should be a pharmaceutically accepted non-toxic salt in order to be used as a medicine, and other salts may, however, be useful in the preparation of the compounds according to the invention or of their non-toxic pharmaceutically acceptable salts.

The pharmaceutically acceptable salts include alkali metal salts such as lithium, sodium or potassium salts; alkaline earth metal such as calcium or magnesium salts; and salts formed with suitable organic ligands such as quaternary ammonium salts. In the case of acid addition salt, for example, a solution of the compound according to the present invention may be mixed with pharmaceutically acceptable non-toxic acid solution such as hydrochloric acid, fumaric acid, maleic acid, succinic acid, acetic acid, citric acid, tartaric acid, carbonic acid or phosphoric acid.

The compounds according to the present invention include prodrugs of the compounds of Formula 1. Generally, such prodrugs will be functional derivatives of the compounds of Formula 1 which are readily converted in vivo into the required compounds. The suitable prodrugs according to the present invention may be selected and prepared by a conventional method [“Design of Prodrugs”, ed. H. Bundgaard, Elsevier, 1985].

The compounds according to the present invention include various tautomers of the compounds of Formula 1.

Where the compounds according to the invention have at least one asymmetric center, they may accordingly exist as enantiomers. Where the compounds according to the invention possess two or more asymmetric centres, they may additionally exist as diastereoisomers. It is to be understood that all such isomers and mixtures thereof are encompassed within the scope of the present invention.

More preferably, the compounds of Formula 1 according to the present invention, a pharmaceutically acceptable salta and prodrug thereof are selected from the group consisting of:

1-Benzyl-7-chloro-3-(4-methoxy-phenyl)-3-methyl-5-(4-methyl-piperazin-1-yl)-1H -quinoline-2,4-dione;

1-Benzyl-7-chloro-3-(4-hydroxy-phenyl)-3-methyl-5-(4-methyl-piperazin-1-yl)-1H-quinoline-2,4-dione;

1-Benzyl-7-chloro-3-methyl-5-(4-methyl-piperazin-1-yl)-3-(4-nitro-phenyl)-1H-quinoline-2,4-dione;

3-(4-Amino-phenyl)-1-benzyl-7-chloro-3-methyl-5-(4-methyl-piperazin-1-yl)-1H-quinoline-2,4-dione;

1-Benzyl-7-chloro-3-(4-diethylamino-phenyl)-3-methyl-5-(4-methyl-piperazin-1-yl)-1H-quinoline-2,4-dione;

1-Benzyl-7-chloro-3-(4-ethylamino-phenyl)-3-methyl-5-(4-methyl-piperazin-1-yl)-1H-quinoline-2,4-dione;

7-Chloro-3-(4-methoxy-phenyl)-3-methyl-5-(4-methyl-piperazin-1-yl)-1-(3-nitro-benzyl)-1H-quinoline-2,4-dione;

7-Chloro-3-(4-hydroxy-phenyl)-3-methyl-5-(4-methyl-piperazin-1-yl)-1-(3-nitro-benzyl)-1H-quinoline-2,4-dione;

1-(3-Amino-benzyl)-7-chloro-3-(4-methoxy-phenyl)-3-methyl-5-(4-methyl-piperazn-1-yl)-1H-quinoline-2,4-dione;

1-(3-Amino-benzyl)-7-chloro-3-(4-hydroxy-phenyl)-3-methyl-5-(4-methyl-piperazin-1-yl)-1H-quinoline-2,4-dione;

1-Benzyl-7-chloro-3-methyl-5-(4-methyl-piperazin-1-yl)-3-phenyl-1H-quinoline-2,4-dione;

1-Benzyl-3-(4-benzyloxy-3-bromo-phenyl)-7-chloro-3-methyl-5-(4-methyl-piperazin-1-yl)-1H-quinoline-2,4-dione;

1-Benzyl-7-chloro-3-(4-methoxy-phenyl)-3-methyl-5-piperazin-1-yl-1H-quinoline-2,4-dione;

(S)-1-Benzyl-7-chloro-3-(4-methoxy-phenyl)-3-methyl-5-piperazin-1-yl-1H-quinoline-2,4-dione;

(R)-1-Benzyl-7-chloro-3-(4-methoxy-phenyl)-3-methyl-5-piperazin-1-yl-1H-quinoline-2,4-dione;

1-Benzyl-7-chloro-3-(4-hydroxy-phenyl)-3-methyl-5-piperazin-1-yl-1H-quinoline-2,4-dione;

(S)-1-Benzyl-7-chloro-3-(4-hydroxy-phenyl)-3-methyl-5-piperazin-1-yl-1H-quinoline-2,4-dione;

(R)-1-Benzyl-7-chloro-3-(4-hydroxy-phenyl)-3-methyl-5-piperazin-1-yl-1H-quinolin-2,4-dione;

1-Benzyl-7-chloro-3-methyl-3-(4-nitro-phenyl)-5-piperazin-1-yl-1H-quinoline-2,4-dione;

3-(4-Amino-phenyl)-1-benzyl-7-chloro-3-methyl-5-piperazin-1-yl-1H-quinoline-2,4-dione;

1-Benzyl-7-chloro-3-(4-diethylamino-phenyl)-3-methyl-5-piperazin-1-yl-1H-quinoline-2,4-dione;

1-Benzyl-7-chloro-3-(4-ethylamino-phenyl)-3-methyl-5-piperazin-1-yl-1H-quinoline-2,4-dione;

1-Benzyl-7-chloro-3-(4-chloro-phenyl)-3-methyl-5-piperazin-1-yl-1H-quinoline-2,4-dione;

1-Benzyl-3-(4-bromo-phenyl)-7-chloro-3-methyl-5-piperazin-1-yl-1H-quinoline-2,4-dione;

1-Benzyl-7-chloro-3-(4-iodo-phenyl)-3-methyl-5-piperazin-1-yl-1H-quinoline-2,4-dione;

1-Benzyl-7-chloro-3-methyl-3-phenyl-5-piperazin-1-yl-1H-quinoline-2,4-dione;

7-Chloro-3-(4-methoxy-phenyl)-3-methyl-1-(3-nitro-benzyl)-5-piperazin-1-yl-1H-quinoline-2,4-dione;

7-Chloro-3-(4-hydroxy-phenyl)-3-methyl-1-(3-nitro-benzyl)-5-piperazin-1-yl-1H-quinoline-2,4-dione;

1-(3-Amino-benzyl)-7-chloro-3-(4-methoxy-phenyl)-3-methyl-5-piperazin-1-yl-1H-quinoline-2,4-dione;

1-(3-Amino-benzyl)-7-chloro-3-(4-hydroxy-phenyl)-3-methyl-5-piperazin-1-yl-1H-quinoline-2,4-dione;

7-Chloro-1-(3-methoxy-benzyl)-3-(4-methoxy-phenyl)-3-methyl-5-piperazin-1-yl-1-H-quinoline-2,4-dione;

7-Chloro-1-(3-hydroxy-benzyl)-3-(4-hydroxy-phenyl)-3-methyl-5-piperazin-1-yl-1H-quinoline-2,4-dione;

7-Chloro-1-(2-methoxy-benzyl)-3-(4-methoxy-phenyl)-3-methyl-5-piperazin-1H-quinoline-2,4-dione;

7-Chloro-1-(2-hydroxy-benzyl)-3-(4-hydroxy-phenyl)-3-methyl-5-piperazin-1-yl-1H-quinoline-2,4-dione;

7-Chloro-1-(4-methoxy-benzyl)-3-(4-methoxy-phenyl)-3-methyl-5-piperazin-1-yl-1H-quinoline-2,4-dione;

7-Chloro-1-(4-hydroxy-benzyl)-3-(4-hydroxy-phenyl)-3-methyl-5-piperazin-1-yl-1H-quinoline-2,4-dione;

1-(3-Bromo-benzyl)-7-chloro-3-(4-methoxy-phenyl)-3-methyl-5-piperazin-1-yl-1H-quinoline-2,4-dione;

1-(3-Bromo-benzyl)-7-chloro-3-(4-hydroxy-phenyl)-3-methyl-5-piperazin-1-yl-1H-quinoline-2,4-dione;

1-(2-Bromo-benzyl)-7-chloro-3-(4-methoxy-phenyl)-3-methyl-5-piperazin-1-yl-1H-quinolin-2,4-dione;

1-(2-Bromo-benzyl)-7-chloro-3-(4-hydroxy-phenyl)-3-methyl-5-piperazin-1-yl-1H-quinoline-2,4-dione;

7-Chloro-3-(4-methoxy-phenyl)-3-methyl-5-piperazin-1-yl-1-pyridin-3-ylmethyl-1H-quinoline-2,4-dione;

7-Chloro-3-(4-hydroxy-phenyl)-3-methyl-5-piperazin-1-yl-1-pyridin-3-ylmethyl-1H-quinoline-2,4-dione;

7-Chloro-3-(4-methoxy-phenyl)-3-methyl-1-naphthalen-2-ylmethyl-5-piperazin-1-yl-1H-quinoline-2,4-dione;

7-Chloro-3-(4-hydroxy-phenyl)-3-methyl-1-naphthalen-2-ylmethyl-5-piperazin-1-yl-1H-quinoline-2,4-dione;

1-Biphenyl-4-ylmethyl-7-chloro-3-(4-methoxy-phenyl)-3-methyl-5-piperazin-1-yl-1H-quinoline-2,4-dione;

1-Biphenyl-4-ylmethyl-7-chloro-3-(4-hydroxy-phenyl)-3-methyl-5-piperazin-1-yl-1H-quinoline-2,4-dione;

1-(1H-Benzoimidazol-2-ylmethyl)-7-chloro-3-(4-methoxy-phenyl)-3-methyl-5-piperazin-1-yl-1H-quinoline-2,4-dione;

7-Chloro-3-(4-methoxy-phenyl)-3-methyl-5-piperazin-1-yl-1-quinolin-2-ylmethyl-1H-quinoline-2,4-dione;

7-Chloro-3-(4-hydroxy-phenyl)-3-methyl-5-piperazin-1-yl-1-quinolin-2-ylmethyl-1H-quinoline-2,4-dione;

7-Chloro-1-ethyl-3-(4-methoxy-phenyl)-3-methyl-5-piperazin-1-yl-1H-quinoline-2,4-dione;

5-Chloro-1-ethyl-3-(4-hydroxy-phenyl)-3-methyl-7-piperazin-1-yl-1H-quinoline-2,4-dione;

7-Chloro-1-cyclohexylmethyl-3-(4-methoxy-phenyl)-3-methyl-5-piperazin-1-yl-1H-quinoline-2,4-dione;

7-Chloro-1-cyclohexylmethyl-3-(4-hydroxy-phenyl)-3-methyl-5-piperazin-1-yl-1H-quinoline-2,4-dione;

1-Benzyl-7-chloro-3-(3-methoxy-phenyl)-3-methyl-5-piperazin-1-yl-1H-quinoline-2,4-dione; and

1-Benzyl-7-chloro-3-(3-hydroxy-phenyl)-3-methyl-5-piperazin-1-yl-1H-quinoline-2,4-dione.

However, the compounds of Formula 1 according to the present invention are not limited to the above-listed compounds.

Additionally, the present invention provides a preparation method of N-substitute-1H-quinoline-2,4-diones represented by Scheme 1 including the steps of:

(a) preparing an intermediate I by a coupling reaction of the compounds 2 and compounds 3;

(b) preparing an intermediate II by cyclization reaction of the compound of the intermediate I in the presence of a base;

(c) preparing an intermediate III by substitution reaction on N(1) of the intermediate II in the presence of a electrophilic group and a base; and

(d) substituting of the intermediate III using a amine.

Additionally, depending on the R¹-, R²- and R⁶-substituents of the Formula 1, specific functional group transformations may be followed next the step (d) of the Scheme 1.

Hereinafter, a preparation method for the N-substituted-1H-quinoline-2,4-diones according to the present invention will be described in detail.

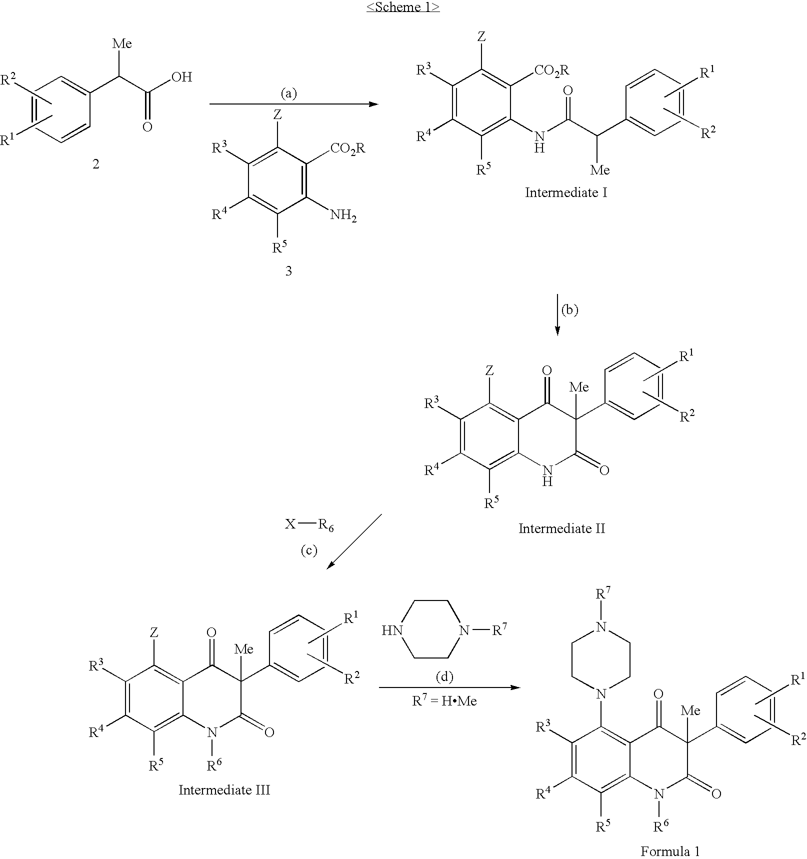

(wherein,

R¹˜R⁷ are same as the aforementioned definition in Formula 1, and R is a methyl, ethyl, or propyl group, and Z represents a halogen such as fluorine, chlorine, bromine and iodine, and X is chlorine, bromine, iodine, o-methylsulfonyl or o-toluenesulfonyl.)

First, in the step (a), the intermediate I may be obtained by coupling reaction of compound 2 and compound 3.

The compound 2 is preferably 2-phenylpropionic acids and the compound 3 is preferably anthranilic acid esters in the present invention, and they may be commercially available or where they are not commercially available, may be prepared by the procedure described herein or by the analogous procedures for known compounds from the art of organic synthesis.

The coupling reaction includes the steps of: 1) forming an acid chloride by reacting the compound 2 with chlorinating agent such as SOCl₂, (COCl)₂, PCl₅, or BOP-Cl (bis(2-oxo-diazolindinyl)phosphinic chloride) in an inert solvent; 2) coupling the acid chloride of a compound 2 and a compound 3 in an inert solvent by mixing and heating them.

The an inert solvent is dichloromethane, 1,2-dichloroethane or methylene chloride. The step 1) may be performed at room temperature and the step 2) may be performed at about 0° C.

Then, in the step (b), cyclization of the intermediates I prepared in step (a) provides the corresponding intermediates II(quinoline-1H-diones) with high yield.

The cyclization is performed under the presence of proper base, and is completed with mild acid[Bioorg. Med. Chem. Lett., 5, 2643(1995); J. Med. Chem., 36, 3386(1993)]. The proper base includes sodium, potassium, sodium hydride, lithium hexamethyldisilazide, and potassium hexamethyldisilazide. Also, the preferable reaction solvent is tetrahydrofuran(THF) and the preferable reaction temperature is −78° C.—reflux temperature.

Then, in the step (c), the intermediate III is obtained by substitution on N(1) of the intermediate II prepared in the above step (b) in the presence of a electrophilic substituent and base.

The introduction of the substituent R⁶ on N(1) of the intermediate II is usually carried out using a electrophilic group, X—R⁶ in the presence of a suitable base such as Na₂CO₃, K₂CO₃ or NaH in aprotic solvent such as acetonitrile, N,N-dimethylformamide etc. at ambient temperature. In this process, X as a leaving group is preferably Cl, Br, I, o-methylsulfonyl, o-toluenesulfonyl etc.

Subsequently, in the step (d), N-substituted-1H-quinoline represented by formula 1 is obtained by reaction of the intermediate II prepared in the step (c) and a appropriate amine.

The appropriate amine is N-methylpiperazine or piperazine and the reaction is nucleophilic substitution reaction of the intermediate III, and the displacement is done using Na₂CO₃, K₂CO₃ in aprotic solvent such as acetonitrile, N,N-dimethylformamide, in only basic solvent like pyridine, or in neat condition at reflux temperature.

And then, after the step (d), depending on the R¹-, R²- and R⁶-substituents of the formula 1, specific functional group transformations may be performed.

A methoxy group may be transformed into a hydroxy group by treatment with a boron tribromide in methylene chloride. A nitro group may be reduced to amino group using tin(II) dihydrate in refluxing protic solvent such as MeOH, EtOH and acetic acid. The reductive alkylation on an amino group may be also performed using the appropriate aldehydes such as formaldehyde, acetaldehyde in the presence of sodium cyanoborohydride as a reducing agent.

Where the above described processes for the preparation of the compounds according to the invention give rise to mixtures of stereoisomers, these isomers may be separated by conventional techniques such as preparative chromatography. The compounds may be prepared in racemic form, or individual enantiomers may be prepared either by asymmetric synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. The present invention extends to cover all structural and optical isomers of the various compounds as well as racemic mixture thereof.

Additionally, the present invention provides a pharmaceutical composition of a 5-HT6 antagonist including the compound of formula 1 and pharmaceutically acceptable salts thereof.

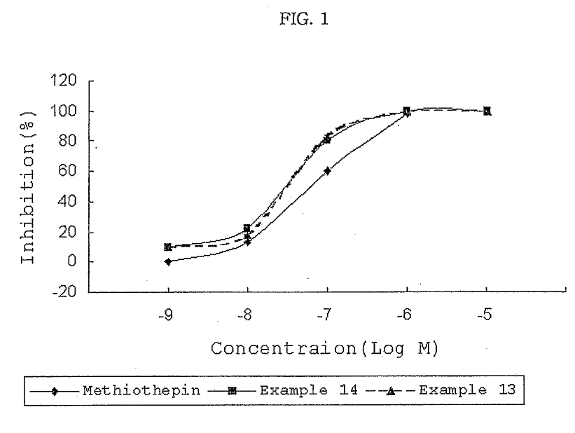

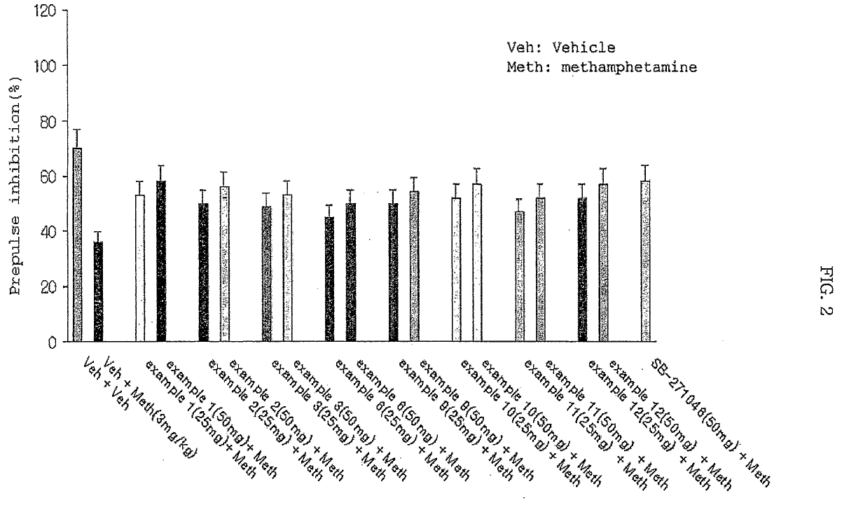

The compound according to the present invention has excellent binding affinity to a serotonin 5-HT6 receptor (Refer to Table 2), excellent selectivity to a 5-HT6 receptor with respect to other receptors (Table 4), and the inhibitory effect on intracellular serotonin(5-HT)-induced cAMP accumulation (FIG. 1) and hyperactivity in rats induced by methamphetamine (2 mg/kg, i.p.) (FIG. 2). In addition to, the compound according to the present invention don't show any rotarod deficit below 400 [symbol] . Therefore, it may be effectively used as a 5-HT6 antagonist.

The 5-HT6 receptor is known to be positively coupled to the adenylyl cyclase system, so agonists of the receptor would increase in a significant way the levels of intracellular cAMP. Thus a substance inhibiting the intracellular serotonin(5-HT)-induced cAMP accumulation may be determined as 5-HT6 receptor antagonist.

The 5-HT6 receptor is known to be positively coupled th the adenylyl cyclase system, so agonists of receptor would increase in a significant way the levels of intracellular cAMP. Thus, a substance inhibiting the intracellular serotonin(5-HT)-induced cAMP accumulation may be determined as a 5HT6 receptor antagonist.

Prepulse inhibition (PPI) of acoustic startle in animals for study the inhibitory effect on hyperactivity in rats is one of the most intensively studied behavioral models with predictive validity for antipsychotic properties of drugs. PPI is an occurrence that reduction or cease of the amplitude of the startle reaction when the main startle stimulus is preceded by the presentation of a weaker stimulus. PPI deficits have been reported in schizophrenic and presumably psychosis-prone subjects [Braff et al., 1992; Simons and Giardina, 1992].

Accordingly, a pharmaceutical composition according to the present invention may be used for treatment 5-HT6 receptor related disorders of the central nervous system, and particularly for cognitive disorders, Alzheimer disease, anxiety, depression, schizophrenia, stress disorder, panic disorder, phobic disorder, obsessive compulsive disorder, post-traumatic-stress syndrome, immune system depression, psychosis, paraphrenia, mania, convulsive disorder, migraine, drug addition, alcoholism, obesity, eating disorder, or sleep disorder.

The compound according to the present invention may be supplied in various formulations such as oral or parenteral administration, or may be preferably administered by intravenous infusion. In pharmaceutical preparation, exipients and diluent such as a filler, bulking agent, binding agent, wetting agent, disintergrant and surfactant may generally be added. The pharmaceutical compositions of the present invention are preferably in unit dosage forms such as tablets, pills, capsules, powders, granules, sterile solutions or suspensions, or suppositories, for oral, intravenous, parenteral or rectal administration. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms. This solid preformulation composition is then subdivided into unit dosage forms of the type described above containing from 0.1 to about 500 [symbol] of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. The liquid forms in which the novel compositions of the present invention may be incorporated for administration orally or by injection include aqueous solutions, syrups, aqueous or oil suspensions, and emulsions with edible oils such as cottonseed oil, sesame oil, coconut oil or peanut oil, as well as elixir and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The preferable dosage level of the pharmaceutical composition of the present invention is about 0.01 to 250 [symbol] per day, preferably about 0.05 to 100 [symbol] per day, and especially about 0.05 to 5 [symbol] per day. The compounds may be administered on a regimen of 1 to 4 times per day. In a particular embodiment, the compounds may be conveniently administered by intravenous infusion.

### Mode for the Invention

Hereinafter, example embodiments of the present invention will be described in detail. Although the following preparation methods and examples are disclosed to illustrate the present invention, this invention should not be construed as limited to the following examples.

### PREPARATION EXAMPLE I

### 1) Intermediate I-1

### 2,4-Dichloro-6-(2-phenyl-propionylamino)-benzoic acid methyl ester

A mixture of 2-phenylpropionic acid (1.35 g, 9.00 mmol) and thionyl chloride (2.34 [symbol] 27.0 mmol) in dichloromethane (15 [symbol] ) was stirred at room temperature for 1 h and then refluxed overnight under a nitrogen atmosphere. The reaction mixture was cooled to room temperature. The resulting solution was concentrated under reduced pressure to give an intermediate acid chloride. Without further purification, the acid chloride was dissolved in dried methylene chloride (15 [symbol] ). To the above solution was added dropwise a solution of methyl 3,5-dichloro anthranilate (1.95 g, 8.88 mmol) in dried methylene chloride (20 [symbol] ) at ice bath. After the 30 min stirring at 0° C., the reaction was warmed up to room temperature and continued to stir overnight. The resulting mixture was diluted with methylene chloride (50 [symbol] ) and washed with water (50 [symbol] ×2), brine (50 [symbol] ×2) and the saturated NaHCO₃ solution, and dried over MgSO₄. After evaporation of the solvent, the residue was purified by a flash chromatography (n-hexane:EtOAc=10:1) to give a title compound (2.88 g, 92%) as a pale yellow oil:

¹H NMR (200 MHz, CDCl₃) δ 1.57 (d, J=7.0 Hz, 3H, CH₃), 3.75 (s, 3H, CO₂CH₃), 3.72 -3.88 (m, 1H, CH), 7.10 (d, J=2.0 Hz, 1H, ArH), 7.24 -7.41 (m, 5H, ArH), 8.38 (d, J=2.0 Hz, 1H, ArH), 8.97 (s, 1H, NH); mp 83-84° C.; MS(EI) m/e 353[M⁺+2], 320, 246, 105; HRMS m/e cacld. for C₁₇H₁₅NO₃Cl₂ 351.0429, found 351.0430.

### 2) Intermediate I-2

### 2,4-Dichloro-6-[2-(4-methoxy-phenyl)-propionylamino]-benzoic acid methyl ester.

The title compound was prepared by the same procedure for the intermediate I-1, using a 2-(4-methoxy-phenyl)-propionic acid (1.45 g, 8.0 mmol), thionyl chloride (2.09 [symbol] 24.0 mmol) and methyl 3,5-dichloroanthranilate (1.54 g, 7.0 mmol). After normal workup, the pure title compound (2.27 g, 85%) was obtained as a slightly yellow syrup by a flash column chromatography (n-hexane:EtOAc=10:1):

¹H NMR (200 MHz, CDCl₃) δ 1.56 (d, J=7.0 Hz, 3H, CH₃), 3.77 (s, 3H, CO₂CH₃), 3.81 (s, 3H, OCH₃), 3.84-3.95 (m, 1H, CH), 6.83-6.97 (m, 2H, ArH), 7.12 (d=2.0 Hz, 1H, ArH), 7.21-7.27 (m, 2H, ArH), 8.41 (d, J=2.0 Hz, 1H, ArH), 8.98 (s, 1H, NH); HRMS(EI) calcd. for C₁₈H₁₇O₄NCl₂ m/e 381.0536[M⁺], found 381.0539.

### 3) Intermediate I-3

### 2,4-Dichloro-6-[2-(4-nitro-phenyl)-propionylamino]-benzoic acid methyl ester.

The title compound was prepared by the same procedure for the intermediate I-1, using a 2-(4-nitro-phenyl)-propionic acid (1.40 g, 7.17 mmol), thionyl chloride (5.1 [symbol] 71.7 mmol) and methyl 3,5-dichloro anthranilate (1.6 g, 5.74 mmol). After normal workup, the pure title compound (2.26 g, 99%) was obtained as a pale yellow solid by a recrystallization from a 1:5 ratio mixture of ethyl acetate and ethyl ether:

¹H NMR (200 MHz, CDCl₃) δ1.63 (d, J=7.1 Hz, 3H, CH₃), 3.85-3.86 (m, 4H, CO₂CH₃ & CH), 7.18 (d, J=2.0 Hz, 1H, ArH), 7.54 (d, J=8.7 Hz, 2H, ArH), 8.24 (d, J=8.7 Hz, 2H, ArH), 8.45 (d, J=2.0 Hz, 1H, ArH), 9.59 (br s, 1H, ArH); mp 148-149° C.;MS(EI) m/e 396[M⁺], 365, 246; HRMS m/e cacld. for C₁₇H₁₄N₂O₅Cl₂ 396.0279, found 396.0276.

### 4) Intermediate I-4

### 2-[2-(4-Bromo-phenyl)-propionylamino]-4,6-dichloro-benzoic acid methyl ester.

The title compound was prepared by the same procedure for the intermediate I-1, using a 2-(4-bromo-phenyl)-propionic acid (11.7 g, 48.3 mmol), thionyl chloride (35.0 [symbol] 480 mmol) and methyl 3,5-dichloroanthranilate (10.1 g, 45.9 mmol). After normal workup, the pure title compound (9.31 g, 55%) was obtained as a white solid by a flash column chromatography (n-hexane:EtOAc=10:1):

¹H NMR (200 MHz, CDCl₃) δ 1.58 (d, J=7.1 Hz, 3H, CH₃), 3.75 (q, J=7.1 Hz, 1H, CH), 3.81(s, 3H, CO₂CH₃), 7.15 (d, J=2.0 Hz, 1H, ArH), 7.20-7.53 (m, 4H, ArH), 8.41 (d, J=1.7 Hz, 1H, ArH), 9.16 (br s, 1H, NH); mp 79-80° C.;MS(EI) m/e 431 [M⁺], 400, 246; HRMS m/e cacld. for C₁₇H₁₄N₁O₃Cl₂Br 430.9728, found 430.9728.

### 5) Intermediate I-5

### 2,4-Dichloro-6-[2-(3-methoxy-phenyl)-propionylamino]-benzoic acid methyl ester.

The title compound was prepared by the same procedure for the intermediate I-1, using a 2-(3-methoxy-phenyl)-propionic acid (3.20 g, 8.40 mmol), thionyl chloride (1.8 [symbol] 25.0 mmol) and methyl 3,5-dichloroanthranilate (1.50 g, 6.70 mmol). After normal workup, the pure title compound (2.10 g, 84%) was obtained as a white solid by a flash column chromatography (n-hexane:EtOAc=5:1):

¹H NMR (200 MHz, CDCl₃) δ 1.59 (d, J=6.8 Hz, 3H, CH₃), 3.66 (q, J=6.8 Hz, 1H, CH), 3.79 (s, 3H, OCH₃), 3.85 (s, 3H, OCH₃), 6.86-6.96 (m, 4H, ArH), 7.15 (d,. J=1.8 Hz, 1H, ArH), 8.42 (d, J=1.8 Hz, 1H, ArH), 8.99 (br s, 1H, NH); MS(EI) m/e 381[M⁺], 246, 214; HRMS m/e cacld. for C₁₈H₁₇NO₄Cl₂ 381.0535, found 381.0541.

### 6) Intermediate I-6

### 2-[2-(4-Benzyloxy-3-bromo-phenyl)-propionylamino]-4,6-dichloro-benzoic acid methyl ester.

The title compound was prepared by the same procedure for the intermediate I-1, using a 2-(4-benzyloxy-3-bromo-phenyl)-propionic acid (1.00 g, 2.98 mmol), thionyl chloride (0.60 [symbol] 8.33 mmol) and methyl 3,5-dichloroanthranilate (0.53 g, 2.41 mmol). After normal workup, the pure title compound (1.06 g, 82%) was obtained as a white solid by a flash column chromatography (n-hexane:EtOAc=5:1):

¹H NMR (200 MHz, CDCl₃) δ 1.62 (d, J=6.4 Hz, 3H, CH₃), 3.66 (q, J=6.4 Hz, 1H, CH), 3.88 (s, 3H, OCH₃), 5.07 (s, 2H, OCH₂Ph), 6.73 (d, J=2.0 Hz, 1H, ArH), 6.81 (d, J=9.0 Hz, 1H, ArH), 6.97 (dd, J=8.6 Hz, 2.4 Hz, 1H, ArH), 7.12 (d, J=2.4 Hz, 1H, ArH), 7.23-7.35 (m, 5H, ArH); 8.68 (br s, 1H, NH); MS(EI) m/e 537[M⁺], 445, 386.

### PREPARATION EXAMPLE II

### 1) Intermediate II-1

### 5,7-Dichloro-3-methyl-3-phenyl-1H-quinoline-2,4-dione

To a precooled (−78 ° C.) solution of the intermediate I-1 (0.82 g, 2.30 mmol) in dry THF (70 [symbol] ) was added dropwise LiHMDS [prepared by treatment of a hexamethyldisilazide (1.47 [symbol] 6.90 mmol) in dry THF (25 [symbol] ) with n-BuLi (3.70 mmol, 2.5M in hexane) at −78° C. for 1 h. The reaction mixture was stirred for 1 h and then refluxed overnight under a nitrogen atmosphere. The reaction was cooled down to room temperature and was quenched by the addition of 1 N HCl aqueous solution. The resulting mixture was extracted with ethyl acetate (150 [symbol] ×3), the organic phase was washed with brine (150 [symbol] ×2) and water (150 [symbol] ×2), dried over MgSOAfter evaporation of the solvent, the residue was purified by a flash chromatography (n-hexane:EtOAc=4:1) to give a title compound (0.57 g, 78%) as a yellowish solid:

¹H NMR (200 MHz, CD₃OD+DMSO-d₆) δ 1.61 (s, 3H, CH₂), 6.96 (m, 1H, ArH), 7.08-7.34 (m, 6H, ArH); m.p. 222-225° C.; MS(EI) m/e 319[M⁺], 285, 132, 104.

### 2) Intermediate II-2

### 5,7-Dichloro-3-(4-methoxy-phenyl)-3-methyl-1H-quinoline-2,4-dione

The title compound was prepared by the same procedure for the intermediate II-1, using the intermediate I-2 (1.44 g, 3.77 mmol) and LiHMDS (11.0 mmol, 1M solution in THF). After normal workup, the pure title compound (0.55 g, 42%; a yellow solid) was obtained by a flash column chromatography (n-hexane:EtOAc=10:1):

¹H NMR (200 MHz, CDCl₃) δ 1.52 (s, 3H, CH₃), 3.67 (s, 3H, CO₂CH₃), 6.89 (d, J=8.9 Hz, 2H, ArH), 6.99-7.08 (m, 3H, ArH), 7.23 (d, J=1.9 Hz, 1H, ArH), 11.25 (s, 1H, NH); m.p. 210-212° C.; MS(EI) m/e 349[M⁺], 162, 134; HRMS m/e cacld. for C₁₇H₁₃NO₃Cl₂ 349.0272, found 349.0278.

### 3) Intermediate II-3

### 5,7-Dichloro-3-methyl-3-(4-nitro-phenyl)-1H-quinoline-2,4-dione

To a suspension of a sodium hydride (50 [symbol] 1.25 mmol, 60% in mineral oil) in dry tetrahydrofuran (20 [symbol] ) was added a solution of the intermediate I-3 (0.20 g, 0.50 mmol) in dry tetrahydrofuran (5 [symbol] ) at 0° C. The reaction mixture was stirred for 5 h. The reaction was quenched by the addition of 0.5M HCl solution (30 [symbol] ). The resulting mixture was extracted with ethyl acetate (50 [symbol] ×3), washed with water (50 [symbol] ×2) and brine (50 [symbol] ×2) and dried over anhydrous MgSO₄. After evaporation of the solvent, the residue was purified by a recrystallization from a 3:1 ratio mixture of methylene chloride and ethyl acetate to provide the pure title compound (0.18 g, 99%) as a pale yellow solid:

¹H NMR (200 MHz, CDCl₃) δ 1.79 (s, 3H, CH₃), 6.80 (d, J=1.8 Hz, 1H, ArH), 7.15 (d, J=1.8 Hz, 1H, ArH), 7.38 (d, J=9.0 Hz, 2H, ArH), 8.18 (d, J=9.0 Hz, 2H), 8.43 (s, 1H, NH); mp 264-265° C.; MS(EI) m/e 364[M⁺]; HRMS m/e cacld. for C₁₆H₁₀ N₂O₄Cl₂ 364.0017, found 364.0010.

### 4) Intermediate II-4

### 3-(4-Amino-phenyl)-5,7-dichloro-3-methyl-1H-quinoline-2,4-dione

To a solution of the intermediate II-3 (1.0 g, 2.74 mmol) in methanol (30 [symbol] ) was added SnCl₂·2H₂O (1.85 g, 8.22 mmol). The resulting solution was stirred at reflux temperature overnight. After the reaction was completed, the solvent was evaporated under reduced pressure to produce a yellow colored residue. The residue was diluted with 1N HCl solution (200 [symbol] ) and was extracted with ethyl acetate (200 [symbol] ×3). The combined organic layer was washed with brine (200 [symbol] ×2) and water (200 [symbol] ×2), and dried over MgSO₄. After evaporation of the solvent, the resulting residue was purified by a flash column chromatography (n-hexane:ethyl acetate=4:1) to produce the pure title compound (0.61 g, 66%) as a pale yellow solid:

¹H NMR (200 MHz, CDCl₃) δ 1.68 (s, 3H, CH₃), 3.68 (br s, 2H, NH₂), 6.58 (d, J=8.8 Hz, 2H, ArH), 6.73 (d, J=1.8 Hz, 1H, ArH), 6.96 (d, J=8.8 Hz, 2H, ArH), 7.08 (d, J=1.8 Hz, 1H, ArH), 8.21 (br s, 1H, NH); mp 226-227° C.; MS(EI) m/e 335[M⁺+1]; HRMS m/e cacld. for C₁₆H₁₂N₂O₂Cl₂ 334.0276, found 334.0282.

### 5) Intermediate II-5

### 5,7-Dichloro-3-(4-iodo-phenyl)-3-methyl-1H-quinoline-2,4-dione

To a solution of the intermediate II-4 (84 [symbol] 0.25 mmol) in dry acetonitrile (10 [symbol] ) was added t-BuONO (50 [symbol] 0.38 mmol) at 0° C. After stirring for 15 min, a CuI₂(119 [symbol] 0.63 mmol) was added and the cold solution was allowed to reach room temperature and then was refluxed for additional 30 min. The resulting suspension was poured into ice water (100 [symbol] ) and was extracted with ethyl acetate (100 [symbol] ×3). The organic layer was washed with water (100 [symbol] ) and brine (100 [symbol] ×2), dried over anhydrous MgSO4, and concentrated in vacuo. The residue was purified by a flash column chromatography (n-hexane:ethyl acetate=5:1) to give the pure title compound (42 [symbol] 38%) as a white solid:

¹H NMR (200 MHz, CDCl₃) δ 1.72 (s, 3H, CH₃), 6.77 (d, J=1.8 Hz, 1H, ArH), 6.91-6.97 (m, 2H, ArH), 7.12 (d, J=1.8 Hz, 1H, ArH), 7.61-7.68 (m, 2H, ArH), 8.37 (br s, 1H, NH); MS(EI) m/e 445[M⁺], 258, 230, 103.

### 6) Intermediate II-6

### 5,7-Dichloro-3-(4-chloro-phenyl)-3-methyl-1H-quinoline-2,4-dione

The title compound was prepared by the same procedure for the intermediate II-5, using the intermediate II-4 (168 mg, 0.50 mmol), t-BuONO (100 [symbol] 0.75 mmol) and CuCl₂ (168 [symbol] 1.25 mmol). After normal workup, the pure title compound (91 [symbol] 52%) was obtained as a white solid by a flash column chromatography (n-hexane:ethyl acetate=5:1):

¹H NMR (200 MHz, CDCl₃) δ 1.73 (s, 3H, CH₃), 6.81 (d, J=1.8 Hz, 1H, ArH), 7.11-7.30 (m, 5H, ArH), 8.82 (br s, 1H, NH); m.p 226-227° C.; MS(EI) m/e 353[M⁺], 318, 187, 166, 138; HRMS m/e cacld. for C₁₆H₁₀NO₂Cl₃ 352.9777, found 352.9764.

### 7) Intermediate II-7

### 3-(4-Bromo-phenyl)-5,7-dichloro-3-methyl-1H-quinoline-2,4-dione

The title compound was prepared by the same procedure for the intermediate II-1, using the intermediate I-4 (400 [symbol] 0.93 mmol) and LiHMDS (2.20 mmol, 1M solution in THF). After normal workup, the pure title compound (240 [symbol] 72%; a white solid) was obtained by a flash column chromatography (n-hexane:EtOAc=5:1):

¹H NMR (200 MHz, CDCl₃+CD₃OD) δ 1.69 (s, 3H, CH₃), 6.91-7.48 (m, 6H, ArH); m.p. 237-238° C.; MS(EI) m/e 397 [M⁺]; HRMS m/e cacld. for C₁₆H₁₀NO₂ Cl₂Br 396.9272, found 396.9268.

### 8) Intermediate II-8

### 5,7-Dichloro-3-(3-methoxy-phenyl)-3-methyl-1H-quinoline-2,4-dione

The title compound was prepared by the same procedure for the intermediate I-1, using the intermediate I-5 (1.0 g, 2.6 mmol) and LiHMDS (7.8 [symbol] 1M solution in THF). After normal workup, the pure title compound (0.55 g, 66%; a white solid) was obtained by a flash column chromatography (n-hexane:EtOAc=5:1) as a white solid:

¹H NMR (200 MHz,CDCl₃) δ 1.74 (s, 3H, CH₃), 3.74 (s, 3H, OCH₃), 6.72-6.82 (m, 4H, ArH), 7.08 (d, J=1.8 Hz, 1H, ArH), 7.18 (dd, J=7.8 Hz, 8.8 Hz, 1H, ArH); m.p. 192-194° C.; MS(EI) m/e 349[M⁺], 335, 315.

### 9) Intermediate II-9

### 3-(4-Benzyloxy-3-bromo-phenyl)-5,7-dichloro-3-methyl-1H-quinoline-2,4-dione

The title compound was prepared by the same procedure for the intermediate II-1, using the intermediate I-6 (0.80 g, 1.49 mmol) and LiHMDS (6.0 [symbol] 1M solution in THF). After normal workup, the pure title compound (0.51 g, 67% a white solid) was obtained by a flash column chromatography (n-hexane:EtOAc=5:1) as a white solid:

¹H NMR (200 MHz,CDCl₃) δ1.71 (s, 3H, CH₃), 5.09 (s, 2H, OCH₂Ph), 6.78-6.79 (d, J=2.0 Hz, 1H, ArH), 6.82-6.87 (d, J=9.0 Hz, 1H, ArH), 7.02-7.07 (dd, J=8.6 Hz, 2.4 Hz, 1H, ArH), 7.11-7.12 (d, J=2.4 Hz, 1H, ArH), 7.33-7.43 (m, 5H, ArH); m.p. 189-190° C. MS(EI) m/e 504 [M⁺+1] HRMS m/e cacld. for C₂₃H₁₆NO₃Cl₂Br 502.9691, found 502.968.

### PREPARATION EXAMPLE III

### 1) Intermediate III-1

### 1-Benzyl-5,7-dichloro-3-(4-methoxy-phenyl)-3-methyl-1H-quinoline-2,4-dione

A mixture of the intermediate II-2 (0.53 g, 1.51 mmol), benzyl bromide (0.2 [symbol] 2.30 mmol) and ₂CO₃(0.63 g, 4.53 mmol) in DMF (15 [symbol] ) was stirred at ambient temperature overnight. The solvent was evaporated under reduced pressure and the residue was suspended with 0.5N HCl aqueous solution (100 [symbol] ). The suspension was extracted with dichloromethane (100 [symbol] ×3). The organic layer was washed with water (100 [symbol] ×2) and brine (100 [symbol] ×2), dried over anhydrous MgSO₄ and evaporated in vacuo. The residue was purified by a recrystallization from dichloromethane to afford the title compound (0.56 g, 84%) as a white solid:

¹H NMR (200 MHz, DMSO-d₆) δ 1.75 (s, 3H, CH₃), 3.68 (s, 3H, OCH₃), 5.30-5.33 (m, 2H, NCH₂Ph), 6.86-6.97 (m, 4H, ArH), 7.14-7.33 (m, 6H, ArH); m.p. 159-160° C.; MS(EI) m/e 439 [M⁺+1]; HRMS m/e cacld. for C₂₄H₁₉NO₃Cl₂ 439.0742, found 439.0738.

### 2) Intermediate III-1-chiral 1

### 3-(S)-1-Benzyl-5,7-dichloro-3-(4-methoxy-phenyl)-3-methyl-1H-quinoline-2,4-dione

The title compound was prepared by the same procedure for the intermediate III-1, using the intermediate II-2-chiral 1 (0.12 g, 0.34 mmol), benzyl bromide (61 [symbol] 0.51 mmol) and K₂CO₃ (0.14 g, 1.0 mmol). After normal workup, the pure title compound (0.11 g, 73%; a white solid) was obtained by a flash column chromatography (n-hexane:EtOAc=8:1):

Analytical data are identical to those of a racemic intermediate III-1, except the melting temperature (m.p. 154-156° C.).

### 3) Intermediate III-1-chiral 2

### 3-(R)-1-Benzyl-5,7-dichloro-3-(4-methoxy-phenyl)-3-methyl-1H-quinoline-2,4-dione

The title compound was prepared by the same procedure for the intermediate III-1, using the intermediate II-2-chiral 2 (0.15g, 0.43 mmol), benzyl bromide (50 [symbol] 0.64 mmol) and K₂CO₃(0.18 g, 1.30 mmol). After normal workup, the pure title compound (0.13 g, 72%; a white solid) was obtained by a flash column chromatography (n-hexane:EtOAc=8:1):

Analytical data are identical to those of a racemic intermediate III-1, except the melting temperature (m.p. 156˜159° C.).

### 4) Intermediate III-2

### 1-Benzyl-5,7-dichloro-3-methyl-3-(4-nitro-phenyl)-1H-quinoline-2,4-dione

The title compound was prepared according to the same procedure as for the intermediate III-1, using the intermediate II-3 (0.37 g, 1.0 mmol), benzyl bromide (143 [symbol] , 1.20 mmol) and K₂CO₃ (0.17 g, 1.20 mmol). After normal workup, the crude was purified by a flash column chromatography (n-hexane:ethyl acetate=6: 1) to afford the pure title compound (0.31 g, 69%) as a white solid:

¹H NMR (200M Hz, CDCl₃) δ1.78 (s, 3H, CH₃), 5.08 (d, J=16.4 Hz, 1H, NCH HPh), 5.45 (d, J=16.4 Hz, 1H, NCHHPh), 6.91 (d, J=1.8 Hz, 1H, ArH), 7.12-7.17 (m, 3H, ArH), 7.26-7.36 (m, 5H, ArH), 8.15 (d, J=9.2 Hz, 2H, ArH); ° C.; MS(EI) m/e 454 [M⁺]; HRMS m/e cacld. for C₂₃H₁₆N₂O₄Cl₂ 454.0487, found 454.0490.

### 5) Intermediate III-3

### 3-(4-Amino-phenyl)-1-benzyl-5,7-dichloro-3-methyl-1H-quinoline-2,4-dione

To a solution of the intermediate III-2 (1.00 g, 2.19 mmol) in MeOH (25 [symbol] ) was added SnCl₂·2H₂O (1.49 g, 6.59 mmol) and the resulting solution was refluxed. After the reaction was completed, the solvent was evaporated under reduced pressure to produce a yellow colored residue. The residue was diluted with 1N HCl solution (100 [symbol] ) and was extracted with ethyl acetate (100 [symbol] ×3). The combined organic layer was washed with brine (100 [symbol] ×2) and water (100 [symbol] ×2), and dried over MgS₄O After evaporation of the solvent, the resulting residue was purified by column chromatography to give the pure title compound (0.50 g, 54%) as a white solid:

¹H NMR (200 MHz, CDCl₃) δ1.68 (s, 3H, CH₃), 3.67 (br s, 2H, NH₂), 4.97 (d, J=16.4 Hz, 1H, NCHHPh), 5.42 (d, J=16.4 Hz, 1H, NCHHPh), 6.54-6.59 (m, 2H, ArH), 6.82-6.90 (m, 3H, ArH), 7.05 (d, J=1.8 Hz, 1H, ArH), 7.13-7.33 (m, 5H, ArH); mp 209-210° C. MS(EI) m/e 424[M⁺], 333, 307, 291; HRMS m/e cacld. for C₂₃ H₁₈N₂O₂Cl₂ 424.0745, found424.0752.

### 6) Intermediate III-4 & III-5

### 1-Benzyl-5,7-dichloro-3-(4-diethylamino-phenyl)-3-methyl-1H-quinoline-2,4-dione (Intermediate III-4).

### 1-Benzyl-5,7-dichloro-3-(4-ethylamino-phenyl)-3-methyl-1H-quinoline-2,4-dione (Intermediate III-5)

To a solution of the intermediate III-3 (1.00 g, 2.35 mmol) in MeOH (25 [symbol] ) was added NaBH₃CN (0.44 g, 7.05 mmol), CH₃CHO (0.49 [symbol] 7.05 mmol) and the acidity of the above mixture was adjusted by addition of acetic acid at pH 5˜6. The resulting solution was allowed to stir at room temperature for 10 hours. The solvent was evaporated under reduced pressure and the resulting residues were diluted with 1M Na₂ CO₃ solution (100 [symbol] ). The aqueous layer was extracted with ethyl acetate (100 [symbol] ×3) and the organic layer was washed with brine (100 [symbol] ×2), dried over anhydrous MgSO₄ and concentrated in vacuo. The residue was purified by flash column chomatography to give two separable title compounds, intermediate III-4 (0.46 g, 41%) and Ei-5 (0.38 g, 36%), as slightly yellow solids:

Intermediate III-4: ¹H NMR (200MHz, CDCl₃) δ1.08 (t, J=6.9 Hz, 6H, 2×NCH CH₃), 1.69 (s, 3H, CH₃), 3.23 (q, J=7.3 Hz, 4H, 2×NCH₂CH₃), 4.97 (d, J=16.4 Hz, 1H, NCHHPh), 5.43 (d, J=16.4 Hz, 1H, NCHHPh), 6.50-6.54 (m, 2H, ArH), 6.80 (d, J=1.8 Hz, 1H, ArH), 6.87-6.93 (m, 2H, ArH), 7.05 (d, J=1.8 Hz, 1H, ArH), 7.12-7.17 (m, 2H, ArH), 7.26-7.32 (m, 3H, ArH); 173-174° C.; MS(EI) m/e 480[M⁺], 465, 391; HRMS m/e cacld. for C₂₇H₂₆NO₂Cl₂ 480.1371, found 480.138,

Intermediate III-5: ¹H NMR (200MHz, CDCl₃) δ1.06 (t, J=6.8 Hz, 3H, NCH₂CH₃), 1.68 (s, 3H, CH₃), 3.04 (m, 2H, NCH₂CH₃), 4.87 (d, J=16.4 Hz, 1H, NCHHPh), 5.42 (d, J=16.4 Hz, 1H, NCHHPh), 6.48-6.52 (m, 2H, ArH), 6.76 (d, J=1.8 Hz, 1H, ArH), 6.84-6.90 (m, 2H, ArH), 7.02 (d, J=1.8 Hz, 1H, ArH), 7.15-7.18 (m, 2H, ArH), 7.25-7.34 (m, 3H, ArH); mp 148-149° C.; MS(EI) m/e 452[M⁺], 437; HRMS m/e cacld. for C₂₅H₂₂N₂O₂Cl₂ 452.1058, found 452.106.

### 7) Intermediate I-6

### 1-Benzyl-5,7-dichloro-3-(4-chloro-phenyl)-3-methyl-1H-quinoline-2,4-dione

The title compound was prepared according to the same procedure as for the intermediate III-1, using the intermedate 11-6 (1.00 g, 2.82 mmol), benzyl bromide (0.51 [symbol] 4.23 mmol) and K₂CO₃ (0.58 g, 4.23 mmol). After normal workup, the crude was purified by a flash column chromatography (n-hexane:ethyl acetate=8:1) to afford the pure title compound (1.04 g, 83%) as a white solid:

¹H NMR (200M Hz, CDCl₃) δ 1.72 (s, 3H, CH₃), 4.99 (d, J=16.5 Hz, 1H, NCH HPh), 5.42 (d, J=16.5 Hz, 1H, NCHHPh), 6.87 (d, J=1.6 Hz, 1H, ArH), 7.02-7.15 (m, 5H, ArH), 7.24-7.34 (m, 5H, ArH); mp 169-170° C.; MS(EI) m/e 443[M⁺], 352, 324; HRMS m/e cacld. for C₂₃H₁₆NO₂Cl₃ 443.0246, found 443.0247.

### 8) Intermediate I-7

### 1-Benzyl-3-(4-bromo-phenyl)-5,7-dichloro-3-methyl-1H-quinoline-2,4-dione

The title compound was prepared according to the same procedure as for the intermediate III-1, using the intermedate II-7 (0.92 g, 2.31 mmol), benzyl bromide (0.41 [symbol] 3.45 mmol) and K₂CO₃ (0.48 g, 3.45 mmol). After normal workup, the crude was purified by a flash column chromatography (n-hexane:ethyl acetate=8:1) to afford the pure title compound (0.77 g, 68%) as a white solid:

¹H NMR (200M Hz, CDCl₃) δ 1.74 (s, 3H, CH₃), 5.01 (d, J=16.8 Hz, 1H, NCH HPh), 5.43 (d, J=16.8 Hz, 1H, NCHHPh), 6.90 (d, J=1.4 Hz, 1H, ArH), 7.11-7.18 (m, 3H, ArH), 7.29-7.48 (m, 5H, ArH); mp 192-193° C.; MS(EI) m/e 489[M⁺+2], 398, 317; HRMS m/e cacld. for C₂₃H₁₆NO₂Cl₂Br 486.9741, found 486.9742.

### Intermediate III-8

### 1-Benzyl-5,7-dichloro-3-(4-iodo-phenyl)-3-methyl-1H-quinoline-2,4-dione

The title compound was prepared according to the same procedure as for the intermediate III-1, using the intermedate III-5 (1.00 g, 2.24 mmol), benzyl bromide (0.40 [symbol] 3.36 mmol) and K₂C₃ O (0.46 g, 3.36 mmol). After normal workup, the crude was purified by a flash column chromatography (n-hexane:ethyl acetate=8:1) to afford the pure title compound (1.10 g, 85%) as a white solid:

¹H NMR (200M Hz, CDCl₃) δ 1.74 (s, 3H, CH₃), 5.01 (d, J=16.6 Hz, 1H, NCH HPh), 5.43 (d, J=16.6 Hz, 1H, NCHHPh), 6.85-6.90 (m, 3H, ArH), 7.11-7.18 (m, 3H, ArH), 7.29-7.39 (m, 3H, ArH), 7.62-7.68 (m, 2H, ArH); mp 195-196° C.; MS(EI) m/e 535[M⁺], 445, 408; HRMS m/e cacld. for C₂₃H₁₆NO₂Cl₁I 534.9603, found 534.960.

### 10) Intermediate III-9

### 1-Benzyl-5,7-dichloro-3-methyl-3-phenyl-1H-quinoline-2,4-dione

The title compound was prepared according to the same procedure as for the intermediate III-1, using the intermediate II-1 (1.00 g, 3.12 mmol), benzyl bromide (0.45 [symbol] 3.75 mmol) and K2CO3 (0.52 g, 3.75 mmol). After normal workup, the crude was purified by a flash column chromatography (n-hexane:ethyl acetate=8:1) to afford the pure title compound (0.96 g, 75%) as a white solid:

¹H NMR (200MHz, CDCl₃) δ 1.74 (s, 3H, CH₃), 4.99 (d, J=16.3 Hz, 1H, NCH HPh), 5.43 (d, J=16.3 Hz, 1H, NCHHPh), 6.83 (d, J=1.6 Hz, 1H, ArH), 7.06-7.33 (m, 11H, ArH); m.p 150-151° C.; MS(EI) m/e 409[M⁺], 396, 375; HRMS m/e cacld. for C₂₃H₁₇NO₂Cl₂ 409.0636, found 409.063

### 11) Intermediate III-10

### 5,7-Dichloro-3-(4-methoxy-phenyl)-3-methyl-1-(3-nitro-benzyl)-1H-quinoline-2,4-dione

The title compound was prepared according to the same procedure as for the intermediate III-1, using the intermediate II-2 (2.20 g, 6.30 mmol), 3-nitrobenzyl bromide (1.62 g, 9.40 mmol) and K₂CO₃ (2.60 g, 19.0 mmol) as a base. After normal workup, the crude was purified by a flash column chromatography (n-hexane:ethyl acetate=3:1) to afford the pure title compound (2.20 g, 73%) as a pale yellow solid:

¹H NMR (200 MHz, CDCl₃) δ 1.71 (s, 3H, CH₃), 3.764 (s, 3H, OCH₃), 4.87 (d, J=16.6 Hz, 1H, CHHPh), 5.72 (d, J=16.6Hz, 1H, NCHHPh), 6.67 (d, J=1.6 Hz, 1H, ArH), 6.81-6.87 (m, 2H, ArH), 7.01-7.06 (m, 2H, ArH), 7.12 (d, J=1.6 Hz, 1H, ArH), 7.48-7.58 (m, 2H, ArH), 8.01 (s, 1H, ArH), 8.14-8.19 (m, 1H, ArH); m.p 161-163° C.; MS(EI) m/e 484[M⁺], 450, 348; HRMS m/e cacld. for C₂₄H₁₈N₂O₅Cl₂ 484.0593, found 484.0595.

### 12) Intermediate III-11

### 1-(3-Amino-benzyl)-5,7-dichloro-3-(4-methoxy-phenyl)-3-methyl-1H-quinoline-2,4-dione

To a solution of the intermediate III-10 (0.50 g, 1.03 mmol) in methanol (20 [symbol] ) was added SnCl₂·2H₂O (0.70 g, 3.10 mmol). The resulting solution was heated to reflux temperature for 4 hr. After the reaction was completed, the solvent was evaporated under reduced pressure to produce a yellow residue. The residue was diluted with 1N HCl solution (100 [symbol] ) and was extracted with ethyl acetate (100 [symbol] ×3). The combined organic layer was washed with brine (100 [symbol] ×2) and water (100 [symbol] ×2), and dried over MgSO₄. After evaporation of the solvent, the resulting residue was purified by a flash column chromatography (n-hexane:ethyl acetate=2:1) to produce the pure title compound (0.23 g, 50%) as a pale yellow solid:

¹H NMR (200 MHz, CDCl₃) δ 1.57 (s, 3H, CH₃), 3.68 (s, 3H, OCH₃), 5.03-5.26 (m, 2H, NCH₂Ph), 6.31-6.45 (m, 3H, ArH), 6.86-6.98 (m, 5H, ArH), 7.12-7.14 J=2.2 Hz, 1H, ArH), 7.34-7.34 (d, J=1.6 Hz, 1H, ArH); m.p 205-206° C.; MS(EI) m/e 454[M⁺], 420, 348; HRMS m/e cacld. for C₂₄H₂₀N₂O₃Cl₂ 454.0851, found 454.0861.

### 13) Intermediate III-12

### 5,7-Dichloro-1-(3-methoxy-benzyl)-3-(4-methoxy-phenyl)-3-methyl-1H-quinoline-2,4-dione

To a suspension of NaH (60% dispersion in mineral oil; 44 [symbol] 1.14 mmol) in dry DMF (7 [symbol] ) was added the intermediate II-2 (0.20 g, 0.57 mmol) in dry DMF (7 [symbol] ) at 0° C. After 30 min stirring, a solution of 3-methoxybenzyl chloride (0.13 g, 0.86 mmol) in dry DMF (2 [symbol] ) by syringe to the above mixture at 0° C. The resulting mixture was allowed to stir at room temperature overnight. After the reaction was completed, the mixture was quenched with cold water (100 [symbol] ) and extracted with ethyl acetate (100 [symbol] ×3). The organic layer was washed with water (100 [symbol] ×2) and brine (100 [symbol] ×2), dried over anhydrous MgSO₄, and evaporated in vacuo. The residue was purified by a flash column chromatography (n-hexane:ethyl acetate=10:1) to afford the title compound (0.18 g, 67%) as a white solid:

¹H NMR (200 MHz, CDCl₃) δ 1.71 (s, 3H, CH₃), 3.43 (s, 6H, 2×OCH₃), 4.92 (d, J=16.6 Hz, 1H, NCHHPh), 5.48 (d, J=16.6 Hz, 1H, NCHHPh), 6.67-6.84 (m, 6H, ArH), 7.02-7.08 (m, 2H, ArH), 7.19-7.27 (m, 1H, ArH).

### 14) Intermediate III-13

### 5,7-Dichloro-1-(2-methoxy-benzyl)-3-(4-methoxy-phenyl)-3-methyl-1H-quinoline-2,4-dione

The title compound was prepared according to the same procedure as for the intermediate III-1, using the intermediate II-2 (0.2 g, 0.57 mmol), 2-methoxybenzyl chloride (0.11 [symbol] 0.74 mmol) and K₂CO₃ (0.24 g, 1.70 mmol) as a base. After normal workup, the crude was purified by a flash column chromatography (n-hexane:ethyl acetate=4: 1) to afford the pure title compound (0.25 g, 93%) as a white solid:

¹H NMR (200 MHz, CDCl₃) δ 1.71 (s, 3H, CH₃), 3.78 (s, 3H, OCH₃), 3.94 (s, 3H, OCH₃), 5.08 (d, J=16.8 Hz, 1H, NCHHPh), 5.38 (d, J=16.8 Hz, 1H, NCHHPh), 6.79-b 6.86 (m, 2H, ArH), 6.88-6.98 (m, 4H, ArH), 7.03-7.11 (m, 3H, ArH), 7.24-7.32 (m, 1H, ArH); mp 170-171° C.; MS(EI) m/e 469[M⁺], 435, 348; HRMS m/e cacld. for C₂₅H₂₁NO₄Cl₂ 469.0848, found469.085.

### 15) Intermediate III-14

### 5,7-Dichloro-1-(4-methoxy-benzyl)-3-(4-methoxy-phenyl)-3-methyl-1H-quinoline-2,4-dione

The title compound was prepared according to the same procedure as for the intermediate III-1, using the intermediate II-2 (1.00 g, 2.85 mmol), 4-methoxybenzyl bromide (0.69 g, 3.43 mmol) and K₂CO₃ (0.47 g, 3.43 mmol) as a base. After normal workup, the crude was purified by a flash column chromatography (n-hexane:ethyl acetate=4:1) to afford the pure title compound (1.11 g, 83%) as a white solid:

¹H NMR (200MHz, CDCl₃) δ1.69 (s, 3H, CH₃), 3.74 (s, 3H, OCH₃) 3.78 (s, 3H, OCH₃), 4.94 (d, J=16.5 Hz, 1H, NCHHPh), 5.40 (d, J=16.5 Hz, 1H, NCHHPh), 6.76-6.88 (m, 3H, ArH), 6.99-7.10 (m, 3H, ArH); m.p 134-135° C.; MS(EI) m/e 469[M⁺]348, 214; HRMS m/e cacld. for C₂₅H₂₁NO₄Cl₂ 469.0848, found 469.0847.

### 16) Intermediate III-15

### 1-(3-Bromo-benzyl)-5,7-dichloro-3-(4-methoxy-phenyl)-3-methyl-1H-quinoline-2,4-dione

The title compound was prepared according to the same procedure as for the intermediate III-1, using the intermediate II-2 (0.2 g, 0.57 mmol), 3-bromobenzyl bromide (0.21 g, 0.85 mmol) and K₂CO₃ (0.24 g, 1.70 mmol) as a base. After normal workup, the crude was purified by a flash column chromatography (n-hexane:ethyl acetate=10: 1) to afford the pure title compound (0.29 g, 96%) as a bright yellow solid:

¹H NMR (200 MHz, CDCl₃) δ 1.70 (s, 3H, CH₃), 3.75 (s, 3H, OCH₃), 4.77 (d, J=16.6 Hz, 1H, NCHPh), 5.54 (d, J=16.6 Hz, 1H, NCHHPh), 6.73 (d, J=1.6 Hz, 1H, ArH), 6.81-6.88 (m, 2H, ArH), 7.01-7.06 (m, 2H, ArH), 7.10 (d, J=1.6 Hz, 1H, ArH), 7.17-7.26 (m, 3H, ArH), 7.41 (d, J=7.8 Hz, 1H, ArH); m.p. 165-166° C.; MS(EI) m/e 519[M⁺], 348, 320.

### 17) Intermediate III-16

### 1-(2-Bromo-benzyl)-5,7-dichloro-3-(4-methoxy-phenyl)-3-methyl-1H-quinoline-2,4-dione

The title compound was prepared according to the same procedure as for the intermediate III-1, using the intermediate II-2 (1.00 g, 2.85 mmol), 2-bromobenzyl bromide (0.86 g, 3.43 mmol) and K₂CO₃ (0.47 g, 3.43 mmol) as a base. After normal workup, the crude was purified by a flash column chromatography (n-hexane:ethyl acetate=10:1) to afford the pure title compound (1.19 g, 81%) as a yellow solid:

¹H NMR (200MHz, CDCl₃) δ1.74 (s, 3H, CH₃), 3.78 (s, 3H, OCH₃), 4.98 (d, J=16.5 Hz, 1H, NCHHPh), 5.45 (d, J=16.5 Hz, 1H, NCHHPh), 6.65 (d, J=1.8 Hz, 1H, ArH), 6.71-6.79 (m, 1H, ArH), 6.83-6.89 (m, 2H, ArH), 7.05-7.19 (m, 5H, ArH), 7.60-7.66 (m, 1H, ArH); m.p 200-201° C.; MS(EI) m/e 519[M⁺], 438, 348; HRMS m/e cacld. for C₂₄H₁₈NO₃Cl₂Br 518.9692, found 518.9692.

### 18) Intermediate III-17

### 5,7-Dichloro-3-(4-methoxy-phenyl)-3-methyl-1-pyridin-3-ylmethyl-1H-quinoline-2,4-dione

The title compound was prepared according to the same procedure as for the intermediate III-1, using the intermediate II-2 (0.20 g, 0.57 mmol), 3-picolyl chloride hydrochloride (0.14 g, 0.86 mmol) and K₂CO₃ (0.24 g, 1.70 mmol) as a base. After normal workup, the crude was purified by a flash column chromatography (nhexane:ethyl acetate=4: 1) to afford the pure title compound (2.20 g, 73%) as a pale yellow solid:

¹H NMR (200 MHz, CDCl₃) δ 1.70 (s, 3H, CH₃), 3.75 (s, 3H, OCH₃), 5.04 (d, J=16.6 Hz, 1H, NCHHPh), 5.43-5.51 (d, J=16.6 Hz, 1H, NCHHPh), 6.72 (d, J=1.6 Hz, 1H, ArH), 6.75-6.84 (m, 2H, ArH), 6.93-7.02 (m, 2H, ArH), 7.11 (d, J=1.6 Hz, 1H, ArH), 7.23-7.26 (m, 1H, ArH), 7.43-7.47 (m, 1H, ArH), 8.53 (m, 2H, ArH); m.p 133-134° C.; MS(EI) m/e 440[M⁺], 348, 134; HRMS m/e cacld. for C₂₃H₁₈N₂O₃Cl₂ 440.0694, found 440.0682.

### 19) Intermediate III-18

### 5,7-Dichloro-3-(4-methoxy-phenyl)-3-methyl-1-naphthalen-2-ylmethyl-1H-quinoline-2,4-dione

The title compound was prepared according to the same procedure as for the intermediate II-12, using the intermediate II-2 (0.20 g, 0.57 mmol), 2-bromomethyl naphthalene (0.19 g, 0.86 mmol) and NaH (60% dispersion in mineral oil; 44 mg, 1.14 mmol) as a base. After normal workup, the crude was purified by a flash column chromatography (n-hexane:ethyl acetate=10:1) to afford the pure title compound (0.16 g, 57%) as a white solid:

¹H NMR (200 MHz, CDCl₃) δ 1.67 (s, 3H, CH₃), 3.73 (s, 3H, OCH₃), 4.99 (d, J=16.6 Hz, 1H, NCHHPh), 5.64-5.72 (d, J=16.6 Hz, 1H, NCHHPh), 6.75-6.82 (m, 3H, ArH), 6.98-7.04 (m, 3H, ArH), 7.25-7.30 (m, 1H, ArH), 7.35-7.45 (m, 3H, ArH), 7.53-7.60 (m, 1H, ArH), 7.75-7.81 (m, 2H, ArH); m.p 232-234° C.; MS(EI) m/e 491[M⁺+2], 381, 348; HRMS m/e cacld. for C₂₈H₂₁N₁O₃Cl₂ 489.0898, found 489.0904.

### 20) Intermediate III-19

### 1-Biphenyl-4-ylmethyl-5,7-dichloro-3-(4-methoxy-phenyl)-3-methyl-1H-quinoline-2,4-dione

The title compound was prepared according to the same procedure as for the intermediate II-12, using the intermediate II-2 (0.10 g, 0.29 mmol), 4-(chloromethyl)biphenyl (0.09 g, 0.43 mmol) and NaH (60% dispersion in mineral oil; 22 [symbol] 0.057 mmol) as a base. After normal workup, the crude was purified by a flash column chromatography (n-hexane:ethyl acetate=10:1) to afford the pure title compound (83 [symbol] 55%) as a pale yellow solid:

¹H NMR (200 MHz, CDCl₃) δ 1.72 (s, 3H, CH₃), 3.75 (s, 3H, OCH₃), 5.04 (d, J=16.4 Hz, 1H, NCHHPh), 5.52 (d, J=16.4 Hz, 1H, NCHHPh), 6.79-6.83 (m, 2H, ArH), 6.88-6.89 (d, J=2.0 Hz, 1H, ArH), 7.02-7.09 (m, 3H, ArH), 7.22 (d, J=8.0 Hz, 2H, ArH), 7.35-7.48 (m, 3H, ArH), 7.52-7.59 (m, 4H, ArH); m.p 170-171° C.; MS(EI) m/e 517[M⁺+2], 515[M⁺], 348, 167; HRMS m/e cacld. for C₃₀H₂₃N₁O₃Cl₂ 515.1055, found 515.1062.

### 21) Intermediate III-20

### 1-(1H-Benzoimidazol-2-ylmethyl)-5,7-dichloro-3-(4-methoxy-phenyl)-3-methyl-1H-quinoline-2,4-dione

The title compound was prepared according to the same procedure as for the intermediate III-1, using the intermediate II-2 (1.00 g, 2.85 mmol), 2-chloromethyl-1H-benzoimidazole (0.57 [symbol] 3.43 mmol) and YCO (0.47 g, 3.43 mmol) as a base. After normal workup, the crude was purified by a flash column chromatography (n-hexane:ethyl acetate=2: 1) to afford the pure title compound (0.93 g, 68%) as a pale yellow solid:

¹H NMR (200MHz, CDCl₃) δ1.74 (s, 3H, CH₃), 3.67 (s, 3H, OCH₃), 5.04 (d, J=16.5 Hz, 1H, NCHHPh), 5.60 (d, J=16.5 Hz, 1H, NCHHPh), 6.65-6.75 (m, 2H, ArH), 6.85-6.93 (m, 2H, ArH), 7.10 (d, J=1.83 Hz, 1H, ArH), 7.25-7.34 (m, 2H, ArH), 7.44-7.48 (m, 1H, ArH), 7.74-7.80 (m, 2H, ArH), 9.85 (br s, 1H, ArH).

### 22) Intermediate III-21

### 5,7-Dichloro-3-(4-methoxy-phenyl)-3-methyl-1-quinolin-2-ylmethyl-1H-quinoline-2,4-dione

The title compound was prepared according to the same procedure as for the intermediate III-1, using the intermediate II-2 (1.00 g, 2.85 mmol), 2-chloromethylquinoline (0.61 g, 3.42 mmol) and K₂CO₃ (0.47 g, 3.43 mmol) as a base. After normal workup, the crude was purified by a flash column chromatography (n-hexane:ethyl acetate=2:1) to afford the pure title compound (1.19 g, 85%) as a pale yellow solid:

¹H NMR (200MHz, DMSO-d₆) δ81.61 (s, 3H, CH₃), 3.72 (s, 3H, OCH₃), 5.32 (d, J=17.2 Hz, 1H, NCHHPh), 5.73 (d, J=17.2 Hz, 1H, NCHHPh), 6.88-6.95 (m, 2H, ArH), 7.19-7.36 (m, 4H, ArH), 7.58-7.64 (m, 2H, ArH), 7.78-7.83 (m, 2H, ArH), 7.98-8.02 (m, 1H, ArH), 8.38-8.47 (m, 1H, ArH); m.p 201-202° C.; MS(EI) m/e 490[M⁺], 456, 348; HRMS m/e cacld. for C₂₇H₂₀N₂O₃Cl₂ 490.0850, found 490.0840.

### 23) Intermediate III-22

### 5,7-Dichloro-1-ethyl-3-(4-methoxy-phenyl)-3-methyl-1H-quinoline-2,4-dione

The title compound was prepared according to the same procedure as for the intermediate III-1, using the intermediate II-2 (0.20 g, 0.57 mmol), bromoethane (64 [symbol] , 0.86 mol) and K₂CO₃ (0.22 g, 1.60 mmol) as a base. After normal workup, the crude was purified by a flash column chromatography (n-hexane:ethyl acetate=8:1) to afford the pure title compound (0.14g, 65%) as a white solid:

¹H NMR (200 MHz,CDCl₃) δ 1.27 (t, J=6.4 Hz, 3H, CH₂CH₃), 1.64 (s, 3H, CH₃), 3.70 (s, 3H, OCH₃), 3.79-4.33 (m, 2H, NCH₂Ph), 6.74-6.80 (m, 2H, ArH), 6.89 (d, J=1.6 Hz, 1H, ArH), 6.93-6.99 (m, 2H, ArH), 7.03 (d, J=1.6 Hz, 1H, ArH); m.p. 152-153° C.; MS(EI) m/e 377[M⁺], 364, 348; HRMS m/e cacld. for C₁₉H₁₇NO₃Cl₂ 377.0584, found 377.058.

### 24) Intermediate III-23

### 5,7-Dichloro-1-cyclohexylmethyl-3-(4-methoxy-phenyl)-3-methyl-1H-quinoline-2,4-dione

The title compound was prepared according to the same procedure as for the intermediate III-1, using the intermediate II-2 (0.2 g, 0.57mmol), bromomethyl cyclohexane (0.54 [symbol] 0.85 mmol) and K₂CO₃ (0.24 g, 1.7 mmol) as a base. After normal workup, the crude was purified by a flash column chromatography (n-hexane:ethyl acetate=10:1) to afford the pure title compound (0.14 g, 65%) as a white solid:

¹H NMR (200 MHz, CDCl₃) δ 1.01-1.28 (m, 5H, cyclohexyl), 1.49-1.55 (m, 1H, cyclohexyl), 1.65 (s, 3H, CH₃), 1.66-1.75 (m, 4H, cyclohexyl), 3.66-3.76 (m, 1H, NCHH), 3.73 (s, 3H, OCH₃), 4.01-4.12 (m, 1H, NCHH), 6.75-6.79 (m, 2H, ArH), 6.89 (d, J=2.4 Hz, 1H, ArH), 6.96-7.00 (m, 2H, ArH), 7.07 (d, J=2.4 Hz, 1H, ArH); m.p. 166-167° C.; MS(EI) m/e 445[M⁺], 411, 349; HRMS m/e cacld. for C₂₄H₂₅HNO₃Cl₂ 445.1211, found 445.1200.

### 25) Intermediate III-24

### 1-Benzyl-5,7-dichloro-3-(3-methoxy-phenyl)-3-methyl-1H-quinoline-2,4-dione

The title compound was prepared according to the same procedure as for the intermediate III-1, using the intermediate II-8 (0.55 g, 1.60 mmol), benzylbromide (0.28 [symbol] 2.40 mmol) and K₂CO₃ (0.66 g, 4.80 mmol) as a base. After normal workup, the crude was purified by a flash column chromatography (n-hexane:ethyl acetate=5:1) to afford the pure title compound (0.48 g, 67%) as a white solid:

¹H NMR (200 MHz, CDCl₃) δ 1.74 (s, 3H, CH₃), 3.71 (s, 3H, OCH₃), 4.95 (d, J=16.8 Hz, 1H, NCHHPh), 5.45 (d, J=16.8 Hz, 1H, NCHHPh), 6.64 (dd, J=2.2, 2.2 Hz, 1H, ArH), 6.68-6.73 (m, 1H, ArH), 6.77-6.82 (m, 1H, ArH), 6.83 (d, J=1.4 Hz, 1H, ArH), 7.07 (d, J=1.4 Hz, 1H, ArH), 7.14-7.20 (m, 3H, ArH), 7.24-7.33 (m, 3H, ArH); m.p. 124-125° C.: MS(EI) m/e 439[M⁺], 411, 348; HRMS m/e cacld. for C₂₄H₁₉ NO₃Cl₂ 439.0742, found 439.07.

### 26) Intermediate III-25

### 1-Benzyl-3-(4-benzyloxy-3-bromo-phenyl)-5,7-dichloro-3-methyl-1H-quinoline-2,4-dione

The title compound was prepared according to the same procedure as for the intermediate III-1, using the intermediate II-9 (0.15 g, 0.30 mmol), benzylbromide (53 [symbol] 0.45 mmol) and K₂CO₃ (124 [symbol] 0.90 mmol) as a base. After normal workup, the crude was purified by a flash column chromatography (n-hexane:ethyl acetate=10:1) to afford the pure title compound (130 [symbol] 72%) as a white solid:

¹H NMR (200 MHz, CDCl₃) δ 1.70 (s, 3H, CH₃), 4.88-4.99 (d, J=16.6 Hz, 1H, NCHHPh), 5.09 (s, 2H, OCH₂Ph), 5.51-5.55 (d, J=16.6 Hz, 1H, NCHHPh), 6.81-6.86 (m, 2H, ArH), 6.95 (dd, J=8.6, 2.4 Hz, 1H, ArH), 7.13-7.17 (m, 3H, ArH), 7.25-7.45 (m, 9H, ArH); m.p. 184-185° C.; MS(EI) m/e 594 [M⁺+1].

### EXAMPLE 1

### 1-Benzyl-7-chloro-3-(4-methoxy-phenyl)-3-methyl-5-(4-methyl-piperazin-1yl)-1H-quinoline-2,4-dione

A mixture of the intermediate III-1 (0.10 g, 0.23 mmol) and 1-methyl piperazine (5.0 [symbol] ) was heated at reflux temperature overnight. The excessively used 1-methyl piperazine was removed in vacuo. The resulting residue was suspended with water (100 [symbol] ) and extracted with ethyl acetate (100 [symbol] ×3). The organic layer was washed with water (100 [symbol] ×2) and brine (100 [symbol] ×2), dried over anhydrous MgS₄O and evaporated in vacuo. The residue was purified by a flash column chromatography (CH₂Cl₂:MeOH=30:1) to provide the pure title compound (0.07 g, 60%) as a yellow solid:

¹H NMR (200 MHz, CDCl₃) δ 1.78 (s, 3H, CH₃), 2.36 (s, 3H, NCH₃), 2.25-2.60 (m, 4H, 2×NCH₂), 2.80-2.91 (m, 2H, 2×NCHH), 3.06-3.17 (m, 2H, 2×NCHH), 3.74 (s, 3H, OCH₃), 5.10 (d, J=16.2 Hz, 1H, NCHHPh), 5.42 (d, J=16.2 Hz, 1H, NCHHPh), 6.61 (d, J=1.6 Hz, 1H, ArH), 6.65 (d, J=1.6 Hz, 1H, ArH), 6.75 (d, J=8.4 Hz, 2H, ArH), 7.04 (d, J=8.4 Hz, 2H, ArH), 7.17-7.36 (m, 5H, ArH); m.p. 66-67° C.; MS(EI) m/e 503 [M⁺]; HRMS m/e cacld. for C₂₉H₃₀N₃O₃Cl₁ 503.1976, found 503.197.

### EXAMPLE 2

### 1-Benzyl-7-chloro-3-(4-hydroxy-phenyl)-3-methyl-5-(4-methyl-piperazin-1-yl)-1H-quinoline-2,4-dione

To a solution of the example 1 (0.14 g, 0.28 mmol) in dichloromethane (5 [symbol] ) was added BBr₃ (0.83 mmol, in 1M dichloromethane) at −78° C. under nitrogen atmosphere. The resulting mixture was allowed to warm up to room temperature. After 4 hours, the reaction mixture was quenched with cold water and extracted with ethyl acetate (100 [symbol] ×3). Combined organic layer was washed with 2N sodium thiosulfate solution (100 [symbol] ), water (100 [symbol] ×2) and brine (100 [symbol] ), dried over anhydrous Mg₄SO and evaporated in vacuo. The residue was purified by a flash column chromatography (CH₂ Cl₂:MeOH=20:1) to provide the pure title compound (75 mg, 55%) as a yellow solid:

¹H NMR (200 MHz, CDCl₃) δ 1.79 (s, 3H, CH₃), 2.40 (s, 3H, NCH₃), 2.62-2.74 (m, 4H, 2×NCH₂), 2.84-3.00 (m, 4H, 2×NCH₂), 5.21 (d, J=16.6 Hz, 1H, NCH HPh), 5.36 (d, J=16.2 Hz, 1H, NCHHPh), 6.56-6.63 (m, 4H, ArH), 6.95-6.99 (d, J=8.6 Hz, 2H, ArH), 7.19-7.36 (m, 5H, ArH); m.p 258-259° C; MS(EI) m/e 489[M⁺], 446, 432; HRMS m/e cacld. for C₂₈H₂₈N₃O₃Cl₁ 489.1819, found 489.1819.

### EXAMPLE 3

### 1-Benzyl-7-chloro-3-methyl-5-(4-methyl-piperazin-1-yl)-3-(4-nitro-phenyl)-1H-quinolin-2,4-dione

The title compound was prepared according to the same procedure as for the example 1, using the intermediate III-2 (57 mg, 0.11 mmol) and 1-methyl piperazine (33 [symbol] 0.33 mmol). After normal workup, the crude was purified by a flash column chromatography (CH₂Cl₂:MeOH=50:1) to afford the pure title compound (32 [symbol] 55%) as a yellow solid:

¹H NMR (200 MHz, CDCl₃) δ 1.83 (s, 3H, CH₃), 2.38 (s, 3H, NCH₃), 2.56-2.74 (m, 4H, NCH₂), 2.86-2.96 (m, 2H, NCH₂), 3.19-3.27 (m, 2H, NCH₂), 5.10 (d, J=16.4 Hz, 1H, NCHHPh), 5.38 (d, J=16.4 Hz, 1H, NCHHPh), 6.65 (d, J=1.6 Hz, 1H, ArH), 6.72 (d, J=1.6 Hz, 1H, ArH), 7.16-7.20 (m, 2H, ArH), 7.25-7.37 (m, 5H, ArH), 8.11 (d, J=9.0 Hz, 2H, ArH); m.p 179-180° C.; MS(EI) m/e 518[M⁺]; HRMS m/e cacld. for C₂₈H₇N₄O₄ Cl 518.1721, found 518.1716.

### EXAMPLE 4

### 3-(4-Amino-phenyl)-1-benzyl-7-chloro-3-methyl-5-(4-methyl-piperazin-1-yl)-1H-quinoline-2,4-dione

The title compound was prepared according to the same procedure as for the example 1, using the intermediate III-3 (85 [symbol] 0.20 mmol) and 1-methyl piperazine (60 [symbol] 0.60 mmol). After normal workup, the crude was purified by a flash column chromatography (CH₂Cl₂:MeOH=20:1) to afford the pure title compound (38 [symbol] 73%) as a yellow solid:

¹H NMR (200 MHz, CDCl₃) δ 1.76 (s, 3H, CH₃), 2.37 (s, 3H, NCH₃), 2.60-2.67(m, 4H, NCH₂), 2.81-2.92 (m, 2H, NCH₂), 3.06-3.16 (m, 2H, NCH₂), 3.65 (br, s, 2H, NH₂), 5.12 (d, J=16.4 Hz, 1H, NCHHPh), 5.42 (d, J=16.4 Hz, 1H, NCHHPh), 6.50-6.65 (m, 4H, ArH), 6.89 (d, J=8.6 Hz, 2H, ArH), 7.19-7.37 (m, 5H, ArH); MS(EI) m/e 488[M⁺].

### EXAMPLE 5

### 1-Benzyl-7-chloro-3-(4-diethylamino-phenyl)-3-methyl-5-(4-methyl-piperazin-1-yl)-1H-quinoline-2,4-dione

To a solution of the intermediate III-4 (1.00 g, 2.07 mmol) in MeCN (20 [symbol] ) was added 1-methyl piperazine (0.62 g, 6.23 mmol) and K₂CO₃ (0.34 g, 2.48 mmol). The resulting solution was allowed to reflux temperature for overnight. The reaction mixture was poured into water (200 [symbol] ) and extracted with ethyl acetate (200 [symbol] ×3). The organic phase was washed with water (200 [symbol] ×2) and brine (200 [symbol] ), dried over anhydrous MgSO₄, and evaporated in vacuo. The residue was purified by flash column chromatography (CH₂Cl₂:CH₃OH=10:1) to give the pure title compound (0.85 g, 75%) as a bright yellow solid:

¹H NMR (200 MHz, CDCl₃) δ1.08 (t, J=6.96 Hz, 6H, 2×NCH₂CH₃), 1.69 (s, 3H, CH₃), 2.37(s, 3H, NCH₃), 2.59-2.64 (m, 4H, CH₂ of piperidine), 2.84-2.90 (m, 2H, CH of piperidine), 3.04-3.10 (m, 2H, CH₂ of piperidine), 3.22 (q, J=7.3 Hz, 4H, 2×NCH₂CH₃), 5.07 (d, J=16.5 Hz, 1H, NCHHPh), 5.38 (d, J=16.5 Hz, 1H, NCHH Ph), 6.46-6.50 (m, 2H, ArH), 6.59-6.62 (m, 2H, ArH), 6.90-6.95 (m, 2H, ArH), 7.23-7.32 (m, 5H, ArH). m.p 204-206° C.; MS(EI) m/e 544[M⁺], 501, 487; HRMS m/e cacld. for C₃₂H₃₇N₄O₂Cl 544.2605, found 544.2611.

### EXAMPLE 6

### 1-Benzyl-7-chloro-3-(4-ethylamino-phenyl)-3-methyl-5-(4-methyl-piperazin-1-yl)-1H-quinoline-2,4-dione

The title compound was prepared according to the same procedure as for the example 5, using the intermediate III-5 (1.00 g, 2.20 mmol) and 1-methyl piperazine (60 [symbol] 0.60 mmol) and K₂CO₃ (0.36 g, 2.64 mmol). After normal workup, the crude was purified by a flash column chromatography (CH₂Cl₂:MeOH=10:1) to afford the pure title compound (0.85 g, 75%) as a pale yellow solid:

¹H NMR (200 MHz, CDCl₃) δ 81.19 (t, J=6.9 Hz, 3H, NCH₂CH₃), 1.78 (s, 3H, CH₃), 2.39 (s, 3H, NCH₃), 2.61-2.66 (m, 4H, CH₂ of piperidine), 2.83-2.93 (m, 2H, CH₂ of piperidine), 3.05-3.15 (m, 4H, CH₂ of piperidine & ArNHCH CH₃), 5.09 (d, J=16.5 Hz, 1H, NCHHPh), 5.39 (d, J=16.5 Hz, 1H, NCHHPh), 6.41-6.49 (m, 2H, ArH), 6.61-6.65 (m, 2H, ArH), 6.89-6.96 (m, 2H, ArH), 7.21-7.39 (m, 5H, ArH). m.p 153-155° C.; MS(EI) m/e 516[M⁺], 473, 459, 446, 368; HRMS m/e cacld. for C₃₀ H₃₃N₄O₂Cl 516.2292, found 516.2287.

### EXAMPLE 7

### 7-Chloro-3-(4-methoxy-phenyl)-3-methyl-5-(4-methyl-piperazin-1-yl)-1-(2-nitro-benzyl)-1H-quinoline-2,4-dione

The title compound was prepared according to the same procedure as for the example 1, using the intermediate III-10 (0.10 g, 0.21 mmol) and 1-methyl piperazine (5 [symbol] ). After normal workup, the crude was purified by a flash column chromatography (CH₂Cl₂:MeOH=20:1) to afford the pure title compound (2.20 g, 73%) as a yellow solid:

¹H NMR (200 MHz, CDCl₃) δ 1.77 (s, 3H, CH₃), 2.37 (s, 3H, NCH₃), 2.58-2.65 (m, 4H, 2×NCH₂), 2.90-2.93 (m, 2H, 2×NCHH), 3.12-3.16 (m, 2H, 2×NCHH), 3.75 (s, 3H, OCH₃), 4.97-5.06 (d, J=17.0 Hz, 1H, NCHHPh), 5.57-5.65 (d, J=17.0 Hz, 1H, NCHHPh), 6.41 (d, J=1.6 Hz, 1H, ArH), 6.70 (d, J=1.6 Hz, 1H, ArH), 6.76-6.81 (m, 2H, ArH), 7.01-7.06 (m, 2H, ArH), 7.50-7.53 (m, 2H, ArH), 8.05 (s, 1H, ArH), 8.14-8.15 (m, 1H, ArH); m.p 171-174° C.; MS(EI) m/e 548[M⁺], 505, 491; HRMS m/e cacld. for C₂₉H₂₉N₄O₅Cl₁ 548.1826, found 548.1826.

### EXAMPLE 8

### 7-Chloro-3-(4-hydroxy-phenyl)-3-methyl-5-(4-methyl-piperazin-1-yl)-1-(3-nitro-benzyl)-1H-quinoline-2,4-dione

The title compound was prepared according to the same procedure as for the example 2, using the example 7 (50 [symbol] 9.10 mmol) and BBr(0.03 mmol, in 1M dichloromethane). After normal workup, the crude was purified by a recrystallization from dichloromethane to afford the pure title compound (25 mg, 51%) as a yellow solid:

¹H NMR (200 MHz, DMSO-d₆) δ 1.63 (s, 3H, CH₃), 2.18 (s, 3H, NCH₃), 2.36-2.38 (m, 4H, 2×NCH₂), 2.83-2.87 (m, 4H, 2×NCH₂), 5.43 (m, 2H, NCH₂Ph), 6.64 (d, J=9.0 Hz, 2H, ArH), 6.74 (dd, J=8.4, 1.6 Hz, 1H, ArH), 6.86-6.90 (m, 3H, ArH), 7.59-7.73 (m, 2H, ArH), 8.11-8.15 (m, 2H, ArH), 9.55 (s, 1H, ArH); m.p 262-264° C.; HRMS me cacld. for C₂₈H₂₇N₄O5Cl 534.1669, found 534.1669.

### EXAMPLE 9

### 1-(3-Amino-benzyl)-7-chloro-3-(4-methoxy-phenyl)-3-methyl-5-(4-methyl-piperazin-1-yl)-1H-quinoline-2,4-dione

To a solution of the intermediate III-11 (0.1 0 g, 0.22 mmol) in pyridine (7 [symbol] ) was added to 1-methyl piperazine (61 [symbol] 0.60 mmol). The reaction mixture was heated at reflux temperature for 2 days. The reaction mixture was poured into water (80 [symbol] ) and extracted with ethyl acetate (100 [symbol] ×3). The organic phase was washed with water (200 [symbol] ×2) and brine (200 [symbol] ), dried over anhydrous MgS₄O and evaporated in vacuo. The residue was purified by a flash column chromatography (CH₂Cl₂:MeOH=10:1) to give the pure title compound (0.076 g, 70%) as a yellow solid:

¹H NMR (200 MHz,CDCl₃) δ 1.78 (s, 3H, CH₃), 2.38 (s, 3H, NCH₃), 2.58-2.74 (m, 4H, 2×NCH₂), 2.83-2.93 (m, 2H, NCHH×2), 3.10-3.21 (m, 2H, NCHH×2), 3.74 (s, 3H, OCH₃), 4.89 (d, J=16.6 Hz, 1H, NCHHPh), 5.33 (d, J=16.6 Hz, 1H, NCHH Ph), 6.37 (m, 1H, ArH), 6.54-6.62 (m, 3H, ArH), 6.66 (d, J=1.6 Hz, 1H, ArH), 6.72-6.80 (m, 2H, ArH), 7.01-7.16 (m, 3H, ArH); m.p. 90-93° C.; MS(EI) m/e 518[M³⁰ ], 476, 461; HRMS m/e cacld. for C₂₉H₃₁N₄O₃Cl 518.2085, found 518.2098.

### EXAMPLE 10

### 1-(3-Amino-benzyl)-7-chloro-3-(4-hydroxy-phenyl)-3-methyl-5-(4-methyl-piperazin-1-yl)-1H-quinoline-2,4-dione

The title compound was prepared according to the same procedure as for the example 2, using the example 9 (0.020 g, 0.039 mmol) and BBr (0.12 mmol, in 1M dichloromethane). After normal workup, the crude was purified by by a flash column chromatography (CH₂Cl₂:MeOH=10:1) to afford the pure title compound (0.015 g, 75%) as a yellow solid:

¹H NMR (200 MHz, CDCl₃+CD₃OD) δ 1.69 (s, 3H, CH₃), 2.31 (s, 3H, NCH₃), 2.54-2.59 (m, 4H, 2×NCH₃), 2.74-2.83 (m, 2H, 2×NCHH), 3.00-3.11 (m, 2H 2×NCHH), 3.17 (br, 2H, NH ₂), 4.79 (d, J=16.2 Hz, 1H, NCHHPh), 5.30 (d, J=16.2 Hz, 1H, NCHHPh), 6.26-6.31 (m, 1H, ArH), 6.50-6.64 (m, 6H, ArH), 6.85-6.91 (m, 2H, ArH), 7.01-7.08 (m, 1H, ArH); decomp. 277° C; MS(EI) m/e 505[M⁺+1], 461, 447; HRMS m/e cacld. for C₂₈H₂₉N₄O₃Cl 504.1928, found 504.1937.

### EXAMPLE 11

### 1-Benzyl-7-chloro-3-methyl-5-(4-methyl-piperazin-1-yl)-3-phenyl-1H-quinoline-2,4-dione

The title compound was prepared according to the same procedure as for the example 5, using the intermediate III-9 (0.5 g, 1.2 mmol) and 1-methyl piperazine (0.44 [symbol] 3.7 mmol) and K₂CO₃ (0.84 g, 6.10 mmol). After normal workup, the crude was purified by a flash column chromatography (CH₂Cl₂:MeOH=10:1) to afford the pure title compound (0.41 g, 73%) as a pale yellow solid:

¹H NMR (200 MHz, CDCl₃) δ 1.82 (s, 3H, CH₃), 2.36 (s, 3H, NCH₃), 2.52-2.62 (m, 4H, 2×NCH₂), 2.81-2.91 (m, 2H, NCH₂), 3.06-3.17 (m, 2H, NCH₂), 5.07 (d, J=16.4 Hz, 1H, NCHHPh), 5.38 (d, J=16.4 Hz, 1H, NCHHPh), 6.59 (d, J=2.2 Hz, 1H, ArH), 6.65 (d, J=2.2 Hz, 1H, ArH), 7.10-7.32 (m, 10H, ArH); m.p 138-140° C.; MS(EI) m/e 473[M⁺]; HRMS m/e cacld. for C₂₈H₂₈N₃O₂Cl 473.1870, found 473.1846.

### EXAMPLE 12

### 1-Benzyl-3-(4-benzyloxy-3-bromo-phenyl)-7-chloro-3-methyl-5-(4-methyl-piperazin-1-yl)-1H-quinoline-2,4-dione

The title compound was prepared according to the same procedure as for the example 5, using the intermediate III-25 (0.11 g, 0.19 mmol) and 1-methyl piperazine (0.05 [symbol] 0.46 mmol) and Cs₂CO₃ (0.176 g, 0.54 mmol). After normal workup, the crude was purified by a flash column chromatography (CH₂Cl₂:MeOH=20:1) to afford the pure title compound (79 mg, 68%) as a pale yellow solid:

¹H NMR (200 MHz, CDCl₃) δ 1.74 (s, 3H, CH₃), 2.32 (s, 3H, NCH₃), 2.60-2.65 (m, 4H, 2×NCH₂), 2.83-2.90 (m, 2H, NCH₂), 3.15-3.19 (m, 2H, NCH₂), 4.96-5.05 (d, J=16.4 Hz, 1H, NCHHPh), 5.19 (s, 2H, OCH₂Ph), 5.41-5.49 (d, J=16.4 Hz, 1H, NCHHPh), 6.59 (d, J=2.4 Hz, 1H, ArH), 6.69-6.70 (d, J=1.6 Hz, 1H, ArH), 6.75-6.79 (d, J=8.6 Hz, 1H, ArH), 6.94-6.99 (dd, J=8.6, 2.4 Hz, 1H, ArH), 7.15-7.41 (m, 1H, ArH); m.p 95-97° C.; MS(EI) m/e 659[M⁺+2].

### EXAMPLE 13

### 1-Benzyl-7-chloro-3-(4-methoxy-phenyl)-3-methyl-5-piperazin-1-yl-1H-quinoline-2,4-dione

The title compound was prepared according to the same procedure as for the example 9, using the intermediate III-1 (0.10 g, 0.23 mmol) and piperazine (0.05 g, 0.57 mmol). After normal workup, the crude was purified by a flash column chromatography (CH₂Cl₂:MeOH=10:1) to provide the pure title compound (0.06 g, 55%) as a yellow solid:

¹H NMR (200 MHz, CDCl₃) δ 1.77 (s, 3H, CH₃), 2.21 (br s,1H, NH), 2.83-2.91 (m, 2H, NCH₂), 3.07-3.16 (m, 4H, 2×NCH₂), 3.48-3.49 (m, 2H, NCH₂), 3.75 (s, 3H, OCH₃), 5.09 (d, J=16.6 Hz, 1H, NCHHPh), 5.42 (d, J=16.6 Hz, 1H, NCHHPh), 6.62-6.66 (m, 2H, ArH), 6.73-6.77 (m, 2H, ArH), 7.00-7.05 (m, 2H, ArH), 7.17-7.33 (m, 2H, ArH); m.p 120-123° C.; MS(EI) m/e 489[M+], 447, 433; HRMS m/e cacld. for C₂₈H₂₈N₃O₃Cl 489.1819, found 489.1806.

### EXAMPLE 13-1: CHIRAL 1 of EXAMPLE 13

### (S)-1-Benzyl-7-chloro-3-(4-methoxy-phenyl)-3-methyl-5-piperazin-1-yl-1H-quinoline-2,4-dione

The title compound was prepared according to the same procedure as for the example 5, using the intermediate III-1-chiral 1 (0.11 g, 0.26 mmol), piperazine (0.11 g, 1.3 mmol) and K₂CO₃ (0.16 g, 1.3 mmol). After normal workup, the crude was purified by a flash column chromatography (CH₂Cl₂:MeOH=10:1) to afford the pure title compound (0.07 g, 61%) as a yellow solid:

Analytical data are identical to those of a racemic example 13.

### EXAMPLE-13-2: CHIRAL 2 of EXAMPLE-13

### (R)-1-Benzyl-7-chloro-3-(4-methoxy-phenyl)-3-methyl-5-piperazin-1-yl-1H-quinoline-2,4-dione

The title compound was prepared according to the same procedure as for the example 5, using the intermediate III-1-chiral 2 (0.20 g, 0.39 mmol), piperazine (80 [symbol] 0.96 mmol) and K₂CO₃ (0.16 g, 1.20 mmol). After normal workup, the crude was purified by a flash Column chromatography (CH₂Cl₂:MeOH=5:1) to afford the pure title compound (0.17 g, 73%) as a yellow solid:

Analytical data are identical to those of a racemic example 13.

### EXAMPLE 14

### 1-Benzyl-7-chloro-3-(4-hydroxy-phenyl)-3-methyl-5-piperazin-1-yl-1H-quinoline-2,4-dione

The title compound was prepared according to the same procedure as for the example 2, using the example 13 (50 [symbol] 0.01 mmol) and BBr(0.03 mmol, in 1M dichloromethane). After normal workup, the crude was purified by a flash column chromatography (CH₂Cl₂:MeOH=10:1) to provide the pure title compound (27 [symbol] 56%) as a yellow solid:

¹H NMR (200 MHz, CD₃OD) δ 1.70 (s, 3H, CH₃), 2.88-2.98 (m, 2H, NCH₃), 3.15-3.32 (m, 6H, 3×NCH₂), 5.16-5.24 (d, J=16.6 Hz, 1H, NCHHPh), 5.35-5.44 (d, J=16.6 Hz, 1H, NCHHPh), 6.63-6.69 (m, 2H, ArH), 6.78-6.82 (m, 2H, ArH), 6.89-6.95 (m, 2H, ArH), 7.14-7.31 (m, 5H, ArH); m.p 197-200° C; MS(EI) m/e 476[M⁺+1]; HRMS m/e cacld. for C₂₇H₂₆N₃O₃Cl 475.1663, found 475.1656.

### EXAMPLE 14-1: CHIRAL 1 OF EXAMPLE 14

### (S)-1-Benzyl-7-chloro-3-(4-hydroxy-phenyl)-3-methyl-5-piperazin-1-yl-1H-quinoline-2,4-dione

The title compound was prepared according to the same procedure as for the example 2, using the example 13-chiral 1 (0.090 g, 0.19 mmol) and BBr (0.56 mmol, in 1M dichloromethane). After normal workup, the crude was purified by a flash column chromatography (CH₂Cl₂:MeOH=5:1) to provide the pure title compound (56 mg, 62%) as a pale yellow solid:

Analytical data are identical to those of a racemic example 14.

### EXAMPLE 14-2: CHIRAL 2 OF EXAMPLE 14

### (R)-1-Benzyl-7-chloro-3-(4-hydroxy-phenyl)-3-methyl-5-piperazin-1-yl-1H-quinoline-2,4-dione

The title compound was prepared according to the same procedure as for the example 2, using the example 13-chiral 2 (0.10 g, 0.21 mmol) and BBr₃ (0.63 mmol, in 1M dichloromethane). After normal workup, the crude was purified by a flash column chromatography (CH₂ Cl₂:MeOH=5:1) to provide the pure title compound (68 [symbol] 68%) as a pale yellow solid:

Analytical data are identical to those of a racemic example 14.

### EXAMPLE 15

### 1-Benzyl-7-chloro-3-methyl-3-(4-nitro-phenyl)-5-piperazin-1-yl-1H-quinoline-2,4-dione

The title compound was prepared according to the same procedure as for the example 5, using the intermediate III-2 (1.00 g, 2.20 mmol), piperazine (0.95 g, 10.9 mmol) and K₂CO₃ (0.91 g, 6.59 mmol). After normal workup, the crude was purified by a flash column chromatography (CH₂Cl₂:MeOH=10:1) to afford the pure title compound (0.83 g, 75%) as a yellow solid:

¹H NMR (200 MHz, CDCl₃) δ 1.81 (s, 3H, CH₃), 2.87 2.96 (m, 2H, NCH₂), 3.01-3.27 (m, 6H, 3×NCH₂), 5.05 (d, J=16.1 Hz, 1H, NCHHPh), 5.36 (d, J=16.1 Hz, 1H, NCHHPh), ArH), 6.67 (dd, J=1.8 Hz, 10.9 Hz, 2H, ArH), 7.15-7.37 (m, 5H, ArH), 8.09 (d, J=8.79 Hz, 2H, ArH); m.p 145-146° C.; MS(EI) m/e 504[M⁺]; HRMS m/e cacld. for C₂₇H₂₅N₄O₄Cl 504.1564, found 504.1566.

### EXAMPLE 16

### 3-(4-Amino-phenyl)-1-benzyl-7-chloro-3-methyl-5-piperazin-1-yl-1H-quinolin-2,4-dione

The title compound was prepared according to the same procedure as for the example 5, using the intermediate III-3 (200 [symbol] 0.47 mmol), piperazine (141 [symbol] 1.41 mmol) and K₂CO₃ (0.19 g, 1.41 mmol). After normal workup, the crude was purified by a flash column chromatography (CH₂Cl₂:MeOH=10:1) to afford the pure title compound (174 [symbol] 78%) as a yellow solid:

¹H NMR (200 MHz, CDCl₃) δ 1.74 (s, 3H, CH₃), 2.54-2.62 (m, 4H, NCH), 2.78-2.82 (m, 2H, NCH₂), 3.12-3.18 (m, 2H, NCH₂), 3.62 (br s, 2H, NH₂), 5.14 (d, J=16.4 Hz, 1H, NCHHPh), 5.39 (d, J=16.4 Hz, 1H, NCHHPh), 6.48-6.62 (m, 4H, ArH), 6.79 (d, J=8.6 Hz, 2H, ArH), 7.20-7.34 (m, 5H, ArH); MS(EI) m/e 474[M⁺].

### EXAMPLE 17

### 1-Benzyl-7-chloro-3-(4-diethylamino-phenyl)-3-methyl-5-piperazin-1-yl-1H-quinoline-2,4-dione

The title compound was prepared according to the same procedure as for the example 5, using the intermediate III-4 (1.00 g, 2.07 mmol), piperazine (0.53 g, 6.23 mmol) and K₂CO₃ (0.34 g, 2.48 mmol). After normal workup, the crude was purified by a flash column chromatography (CH₂Cl₂:MeOH=10:1) to afford the pure title compound (0.82 g, 75%) as a pale yellow solid:

¹H NMR (200 MHz, CDCl₃) δ1.07 (t, J=6.9 Hz, 6H, 2×NCH₂CH₃), 1.76 (s, 3H, CH₃), 2.85-2.93 (m, 2H, NCH₂), 3.07-3.18 (m, 6H, 3×NCH₂), 3.22 (q, J=7.3 Hz, 4H, 2×NCH₂CH₃), 5.06 (d, J=16.5 Hz, 1H, NCHHPh), 5.39 (d, J=16.5 Hz, 1H, NCHHPh), 6.46-6.52 (m, 2H, ArH), 6.60-6.64 (m, 2H, ArH), 6.87-6.95 (m, 2H, ArH), 7.19-7.36 (m, 5H, ArH); decomp. 195° C.; MS(EI) m/e 530[M⁺], 501, 487;

HRMS m/e cacld. for C₃₁H₃₅N₄O₂Cl 530.2448, found 530.2445.

### EXAMPLE 18

### 1-Benzyl-7-chloro-3-(4-ethylamino-phenyl)-3-methyl-5-piperazin-1-yl-1H-quinoline-2,4-dione

The title compound was prepared according to the same procedure as for the example 5, using the intermediate III-5 (1.00 g, 2.20 mmol), piperazine (0.57 g, 6.62 mmol) and K ₂CO₃ (0.36 g, 2.64 mmol). After normal workup, the crude was purified by a flash column chromatography (CH₂Cl₂:MeOH=10:1) to afford the pure title compound (0.83 g, 76%) as a pale yellow solid:

1H NMR (200 MHz, CDCl₃) δ1.17 (t, J=6.9 Hz, 3H, NCH₂CH₃), 1.76 (s, 3H, CH₃), 2.87-2.91 (m, 4H, 2×NCH₂), 3.02-3.15 (m, 7H, 3×NCH₂ & NH), 5.06 (d, J=16.5 Hz, 1H, NCHHPh), 5.37 (d, J=16.5 Hz, 1H, NCHHPh), 6.40-6.46 (m, 2H, ArH), 6.62-6.65 (m, 2H, ArH), 6.85-6.90 (m, 2H, ArH), 7.18-7.37 (m, 5H, ArH); m.p 236 238° C.; MS(EI) m/e 502[M⁺], 472, 459, 368; HRMS m/e cacld. for C₂₉H₃₁N₄O₂ Cl 502.2135, found 502.2149.

### EXAMPLE 19

### 1-Benzyl-7-chloro-3-(4-chloro-phenyl)-3-methyl-5-piperazin-1-yl-1H-quinoline-2,4-dione

The title compound was prepared according to the same procedure as for the example 5, using the intermediate III-6 (1.00 g, 2.25 mmol), piperazine (0.97 g, 11.24 mmol) and K₂CO₃ (0.93 g, 6.74 mmol). After normal workup, the crude was purified by a flash column chromatography (CH₂Cl₂:MeOH=10:1) to afford the pure title compound (0.83 g, 75%) as a pale yellow solid:

¹H NMR (200 MHz, CDCl₃) δ81.77 (s, 3H, CH₃), 2.85-2.93 (m, 2H, NCH₂), 3.03-3.18 (m, 6H, 3×NCH₂), 5.03 (d, J=16.2 Hz, 1H, NCHHPh), 5.39 (d, J=16.2 Hz, 1H, NCHHPh), 6.63-6.69 (m, 2H, ArH), 7.01-7.05 (m, 2H, ArH), 7.16-7.38 (m, 5H, ArH); m.p 135-136° C.; MS(EI) m/e 493[M⁺]; HRMS m/e cacld. for C₂₇H₂₅N₂O₂Cl₂ 2493.1324, found 493.1325.

### EXAMPLE 20

### 1-Benzyl-3-(4-bromo-phenyl)-7-chloro-3-methyl-5-piperazin-1-yl-1H-quinoline-2,4-dione

The title compound was prepared according to the same procedure as for the example 5, using the intermediate III-7 (1.00 g, 2.04 mmol), piperazine (0.88 g, 10.22 mmol) and K₂CO₃ (0.85 g, 6.13 mmol). After normal workup, the crude was purified by a flash column chromatography (CH₂Cl₂:MeOH=10:1) to afford the pure title compound (0.79 g, 72%) as a yellow solid:

¹H NMR (200MHz, CDCl₃) δ1.77 (s, 3H, CH₃), 2.80 2.92 (m, 2H, NCH₂), 3.02-3.20 (m, 6H, 3×NCH₂), 5.02 (d, J=16.2 Hz, 1H, NCHHPh), 5.37 (d, J=16.2 Hz, 1H, NCHHPh), 6.63 (m, 2H, ArH), 6.95 (m, 2H, ArH), 7.15-7.38 (m, 7H, ArH); m.p 116-117° C.; MS(EI) m/e 538[M⁺+1], 537[M⁺]; HRMS m/e cacld. for C₂₇H₂₅N₃O₂ClBr 537.0818, found 537.0818.

### EXAMPLE 21

### 1-Benzyl-7-chloro-3-(4-iodo-phenyl)-3-methyl-5-piperazin-1-yl-1H-quinoline-2,4-dione

The title compound was prepared according to the same procedure as for the example 5, using the intermediate III-8 (1.00 g, 1.87 mmol), piperazine (0.80 g, 9.35 mmol) and K₂CO₃ (0.77 g, 5.61 mmol). After normal workup, the crude was purified by a flash column chromatography (CH₂Cl₂:MeOH=10:1) to afford the pure title compound (0.85 g, 78%) as a yellow solid:

¹H NMR (200MHz, CDCl₃) δ 1.76 (s, 3H, CH₃), 2.29 (s, 1H, NH), 2.78-2.89 (m, 2H, NCH₂), 2.96-3.17 (m, 6H, 3×NCH₂), 5.02 (d, J=16.2 Hz, 1H, NCHHPh), 5.36 (d, J=16.2 Hz, 1H, NCHHPh), 6.62 (d, J=1.6 Hz, 1H, ArH), 6.68 (d, J=1.6 Hz, 1H, ArH), 6.83 (m, 2H, ArH), 7.15-7.19 (m, 2H, ArH), 7.21-7.37 (m, 3H, ArH), 7.54 (m, 2H, ArH); m.p 176-177° C.; MS(EI) m/e 585[M⁺], 555, 543; HRMS m/e cacld. for C₂₇ H₂₅N₃O₂ClI 585.0680, found 585.0675.

### EXAMPLE 22

### 1-Benzyl-7-chloro-3-methyl-3-phenyl-5-piperazin-1-yl-1H-quinoline-2,4-dione

The title compound was prepared according to the same procedure as for the example 5, using the intermediate III-9 (0.5 g, 1.20 mmol), piperazine (0.31 g, 3.70 mmol) and K₂CO₃ (0.84 g, 6.10 mmol). After normal workup, the crude was purified by a flash column chromatography (CH₂Cl₂:MeOH=10:1) to provide the pure title compound (0.41 g, 73%) as a yellow solid:

¹H NMR (200 MHz, CDCl₃) δ 1.84 (s, 3H, CH₃), 2.78-2.88 (m, 2H, NCH₂), 3.06-3.12 (m, 6H, 3×NCH₂), 5.10 (d, J=16.2 Hz, 1H, NCHHPh), 5.41 (d, J=16.2 Hz, 1H, NCHHPh), 6.62 (d, J=1.6 Hz, 1H, ArH), 6.67 (d, J=1.6 Hz, 1H, ArH), 7.13-7.35 (m, 10H, ArH); MS(EI) m/e 459[M⁺]; HRMS m/e cacld. for C₂₇H₂₆N₃O₂Cl 459.1714, found 459.1704.

### EXAMPLE 23

### 7-Chloro-3-(4-methoxy-phenyl)-3-methyl-1-(3-nitro-benzyl)-5-piperazin-1-yl-1H quinoline-2,4-dione

The title compound was prepared according to the same procedure as for the example 5, using the intermediate III-10 (1.00 g, 2.06 mmol), piperazine (0.89 g, 10.3 mmol) and K₂CO ₃(0.85 g, 6.18 mmol). After normal workup, the crude was purified by a flash column chromatography (CH₂Cl₂:MeOH=10:1) to provide the pure title compound (0.83 g, 75%) as a yellow solid:

¹H NMR (200 MHz, CDCl₃) δ 1.77 (s, 3H, CH₃), 2.85-2.93 (m, 2H, NCH₂), 303-3.23 (m, 6H, 3×NCH₂), 4.96 (d, J=16.8 Hz, 1H, NCHHPh), 5.59 (d, J=16.8 Hz, 1H, NCHHPh), 6.71 (d, J=1.8 Hz, 1H, ArH), 6.76-6.82 (m, 3H, ArH), 7.01 (m, 2H, ArH), 7.51 (m, 2H, ArH), 8.05 (s, 1H, ArH), 8.13-8.19 (m, 1H, ArH); m.p 153-154° C.; MS(EI) m/e 534[M⁺], 517, 504, 492; HRMS m/e cacld. for C₂₈H₂₇N₄O₅Cl 534.1670, found 534.1675.

### EXAMPLE 24

### 7-Chloro-3-(4-hydroxy-phenyl)-3-methyl-1-(3-nitro-benzyl)-5-piperazin-1-yl-1H-quinoline-2,4-dione

The title compound was prepared according to the same procedure as for the example 2, using the example 23 (0.20 g, 0.04 mmol) and BBr (0.12 mmol, in 1M dichloromethane). After normal workup, the crude was purified by a flash column chromatography (CH₂Cl₂:MeOH=10:1) to provide the pure title compound (0.16 g, 81%) as a yellow solid:

¹H NMR (200 MHz, CD₃OD) δ 1.73 (s, 3H, CH₃), 2.85-2.93 (m, 2H, NCH₂), 3.07-3.21 (m, 6H, 3×CH₂), 5.25 (d, J=16.6 Hz, 1H, NCHHPh), 5.53 (d, J=16.6 Hz, 1H, NCHHPh), 6.65-6.75 (m, 3H, ArH), 6.85 (d, J=1.6 Hz, 1H, ArH), 6.91-6.99 (m, 2H, ArH), 7.56-7.68 (m, 2H, ArH), 8.15-8.20 (m, 2H, ArH); m.p 252-253° C.; MS(EI) m/e 520[M⁺], 496, 478; HRMS m/e cacld. for C₂₇H₂₅N₄O₅Cl 520.1513, found 520.1510.

### EXAMPLE 25

### 1-(3-Amino-benzyl)-7-chloro-3-(4-methoxy-phenyl)-3-methyl-5-piperazin-1yl-1H-quinoline-2,4-dione

The title compound was prepared according to the same procedure as for the example 5, using the intermediate III-11 (1.00 g, 2.20 mmol), piperazine (0.95 g, 10.98 mmol) and K₂CO₃ (0.91 g, 6.59 mmol). After normal workup, the crude was purified by a flash column chromatography (CH₂Cl₂:MeOH=10:1) to provide the pure title compound (0.81 g, 73%) as a yellow solid:

¹H NMR (200 MHz, CDCl₃) δ 1.76 (s, 3H, CH₃), 2.87-2.96 (m, 2H, NCH₂), 3.16-3.27 (m, 6H, 3×NCH₂), 3.59 (br s, 2H, NH₂), 3.74 (s, 3H, OCH₃), 4.89 (d, J=16.1 Hz, 1H, NCHHPh), 5.30 (d, J=16.1 Hz, 1H, NCHHPh), 6.37 (d, J=1.8 Hz, 1H, ArH), 6.53-6.61 (m, 2H, ArH), 6.65 (m, 2H, ArH), 6.74 (m, 2H, ArH), 7.01-7.14 (m, 3H, ArH); m.p 183-184° C.; MS(EI) m/e 504[M⁺]; HRMS m/e cacld. for C₂₈H₂₉N₄ O₃Cl 504.1928, found 504.1925.

### EXAMPLE 26

### 1-(3-Amino-benzyl)-7-chloro-3-(4-hydroxy-phenyl)-3-methyl-5-piperazin-1yl-1H-quinoline-2,4-dione

The title compound was prepared according to the same procedure as for the example 2, using the example 25 (0.20 g, 0.39 mmol) and BBr (1.19 mmol, in 1M dichloromethane). After normal workup, the crude was purified by a flash column chromatography (CH₂Cl₂:MeOH=8:1) to provide the pure title compound (0.17 g, 87%) as a yellow solid:

¹H NMR (200 MHz, CDCl₃) δ 1.68 (s, 3H, CH₃), 2.80-2.94 (m, 2H, NCH₂), 3.10-3.2 (m, 6H, 3×NCH₂), 3.54 (br s, 2H, NH₂), 4.79 (d, J=16.2 Hz, 1H, NCHHPh), 5.30 (d, J=16.2 Hz, NCHHPh), 6.31 (d, J=1.8 Hz, 1H, ArH), 6.50-6.62 (m, 2H, ArH), 6.65-6.85 (m, 2H, ArH), 6.94 (m, 2H, ArH), 7.01-7.14 (m, 3H, ArH); MS(EI) m/e 490[M⁺].

### EXAMPLE 27

### 7-Chloro-1-(3-methoxy-benzyl)-3-(4-methoxy-phenyl)-3-methyl-5-piperazin-1-yl-1H-quinoline-2,4-dione

The title compound was prepared according to the same procedure as for the example 9, using the intermediate III-12 (0.39 g, 0.83 mmol) and piperazine (0.11 g, 1.1 mmol). After normal workup, the crude was purified by a flash column chromatography (CH₂Cl₂:MeOH=10:1) to provide the pure title compound (0.24 g, 56%) as a yellow solid:

¹H NMR(200 MHz, CDCl₃) δ 1.76 (s, 3H, CH₃), 2.70 (br s, 1H, NH), 2.85-2.93 (m, 1H, NCHH), 3.05-3.20 (m, 7H, 3 NCH₂, NCHH), 3.73 (s, 3H, OCH₃), 3.74 (s, 3H, OCH₃), 4.96 (d, J=16.2 Hz, 1H, NCHHPh), 5.39 (d, J=16.2 Hz, 1H, NCHHPh), 6.63-6.68 (m, 2H, ArH), 6.70-6.83 (m, 5H, ArH), 6.99-7.07 (m, 2H, ArH), 7.19-7.27 (m, 1H, ArH); m.p 110-112° C.; MS(EI) m/e 519[M⁺], 490, 477; HRMS m/e cacld. for C₂₉H₃₀N₃O₄Cl 519.1925, found 519.1947.

### EXAMPLE 28

### 7-Chloro-1-(3-hydroxy-benzyl)-3-(4-hydroxy-phenyl)-3-methyl-5-piperazin-1-yl-1H-quinoline-2,4-dione

The title compound was prepared according to the same procedure as for the example 2, using the example 27 (0.030 g, 0.058 mmol) and BBr (0.15 mmol, in 1M dichloromethane). After normal workup, the crude was purified by a flash column chromatography (CH₂Cl₂:MeOH=5:1) to provide the pure title compound (15 mg, 52%) as a pale yellow solid:

¹H NMR(200 MHz, CDCl₃+CD₃OD) δ 1.59 (s, 3H, CH₃), 2.75-2.86 (m, 2H, NCH₂), 3.02-3.18 (m, 5H, 2 NCH₂ & NCHH), 3.21-3.23 (m, 1H, NCHH), 4.98 (d, J=16.0 Hz, 1H, NCHHPh), 5.13 (d, J=16.0 Hz, 1H, NCHHPh), 6.52-6.61 (m, 7H, ArH), 6.75-6.80 (m, 2H, ArH), 6.97-7.30 (m, 2H, ArH); m.p 229-230° C.; MS(EI) m/e 491 [M⁺], 461, 449; HRMS m/e cacld. for C₂₇H₂₆N₃O₄Cl 491.1611, found 491.1615.

### EXAMPLE 29

### 7-Chloro-1-(2-methoxy-benzyl)-3-(4-methoxy-phenyl)-3-methyl-5-piperazin-1-yl-1H-quinoline-2,4-dione

The title compound was prepared according to the same procedure as for the example 5, using the intermediate III-13 (0.22 g, 0.47 mmol), piperazine (0.10 g, 1.20 mmol) and K₂CO₃(0.33 g, 2.40 mmol). After normal workup, the crude was purified by a flash column chromatography (CH₂Cl₂:MeOH=10:1) to afford the pure title compound (0.18 g, 74%) as a yellow solid:

¹H NMR (200 MHz,CDCl₃) δ 1.81 (s, 3H, CH₃), 2.82-2.90 (m, 2H, NCH₂), 3.06-3.09 (m, 6H, NCH₂×3), 3.77 (s, 3H, OCH₃), 3.95 (s, 3H, OCH₃), 5.12 (d, J=16.4 Hz, 1H, NCHHPh), 5.38 (d, J=16.4 Hz, 1H, NCHHPh), 6.65 (d, J=2.4 Hz, 1H, ArH), 6.73 (d, J=2.4 Hz, 1H, ArH), 6.75-6.80 (m, 2H, ArH), 6.85-6.98 (m, 3H, ArH), 7.04-7.10 (m, 2H, ArH), 7.22-7.31 (m, 1H, ArH); m.p. 153-155° C.; MS(EI) m/e 519 [M⁺], 502, 489, 477; HRMS m/e cacld. for C₂₉H₃₀N₃O₄Cl 519.1925, found 519.1930.

### EXAMPLE 30

### 7-Chloro-1-(2-hydroxy-benzyl)-3-(4-hydroxy-phenyl)-3-methyl-5-piperazin-1-yl-1H-quinoline-2,4-dione

The title compound was prepared according to the same procedure as for the example 2, using the example 29 (0.074 g, 0.14 mmol) and BBr (0.43 mmol, in 1M dichloromethane). After normal workup, the crude was purified by a flash column chromatography (CH₂Cl₂:MeOH=5:1) to provide the pure title compound (37 mg, 52%) as a pale yellow solid:

¹H NMR (200 MHz, CDCl₃+CD₃OD) δ 1.77 (s, 3H, CH₃), 2.78-2.84 (m, 2H, NCH₂), 2.99-3.02 (m, 6H, 3×NCH₂), 5.14 (d, J=16.4 Hz, 1H, NCHHPh), 5.30 (d, J=16.4 Hz, 1H, NCHHPh), 6.65-6.69 (m, 2H, ArH), 6.75-6.84 (m, 2H, ArH), 6.88-6.97 (m, 3H, ArH), 7.02-7.18 (m, 2H, ArH); m.p. 165-167° C.

### EXAMPLE 31

### 7-Chloro-1-(4-methoxy-benzyl)-3-(4-methoxy-phenyl)-3-methyl-5-piperazin-1-yl-1H-quinoline-2,4-dione

The title compound was prepared according to the same procedure as for the example 5, using the intermediate III-14 (1.00 g, 2.12 mmol), piperazine (0.55 g, 6.37 mmol) and K₂CO₃(0.88 g, 6.37 mmol). After normal workup, the crude was purified by a flash column chromatography (CH₂Cl₂:MeOH=10:1) to afford the pure title compound (0.86 g, 78%) as a pale yellow solid:

¹H NMR (200 MHz, CDCl₃) δ1.78 (s, 3H, CH₃), 2.74-2.90 (m, 4H, 2×NCH₂), 3.02-3.13 (m, 4H, 2×NCH₂), 3.75 (s, 3H, OCH₃) 3.80 (s, 3H, OCH₃), 5.01 (d, J=16.4 Hz, 1H, NCHHPh), 5.31 (d, J=16.4 Hz, 1H, NCHHPh), 6.66-6.67 (m, 2H, ArH), 6.72-6.79 (m, 2H, ArH), 6.82-6.89 (m, 2H, ArH), 6.99-7.07 (m, 2H, ArH), 7.13-7.28 (m, 2H, ArH); m.p. 182-183° C.; MS(EI) m/e 519[M⁺], 489, 477, 357; HRMS m/e cacld. for C₂₉H₃₀N₃O₄Cl 519.1924, found 519.1926.

### EXAMPLE 32

### 7-Chloro-1-(4-hydroxy-benzyl)-3-(4-hydroxy-phenyl)-3-methyl-5-piperazin-1-yl-1H-quinoline-2,4-dione

The title compound was prepared according to the same procedure as for the example 2, using the example 31 (1.00 g, 1.92 mmol) and BBr₃ (5.77 mmol, in 1M dichloromethane). After normal workup, the crude was purified by a flash column chromatography (CH₂Cl₂:MeOH=5:1) to provide the pure title compound (0.75 g, 79%) as a pale yellow solid:

¹H NMR (200 MHz, CD₃OD) δ1.72 (s, 3H, CH₃), 2.77-2.92 (m, 2H, NCH₂), 2.97-3.10 (m, 6H, 3×NCH₂), 5.08 (d, J=16.1 Hz, 1H, NCHHPh), 5.25 (d, J=16.1 Hz, 1H, NCHHPh), 6.63-6.82 (m, 6H, ArH), 6.90-6.98 (m, 2H, ArH), 7.05-7.10 (m, 2H, ArH); m.p 241-242° C.; MS(EI) m/e 491[M⁺]; HRMS m/e cacld. for C₂₇H₂₆N₃O₄Cl 491.1612, found 491.1612.

### EXAMPLE 33

### 1-(3-Bromo-benzyl)-7-chloro-3-(4-methoxy-phenyl)-3-methyl-5-piperazin-1-yl-1H-quinoline-2,4-dione

The title compound was prepared according to the same procedure as for the example 5, using the intermediate III-15 (0.2 g, 0.39 mmol), piperazine (80 [symbol] 0.96 mmol) and K₂CO₃ (0.16 g, 1.20 mmol). After normal workup, the crude was purified by a flash column chromatography (CH₂Cl₂:MeOH=10:1) to afford the pure title compound (0.15 g, 65%) as a pale yellow solid:

¹H NMR (200 MHz,CDCl₃) δ 1.79 (s, 3H, CH₃), 2.83-2.92 (m, 2H, NCH₂), 3.09-3.22 (m, 6H, 3×NCH₂), 3.77 (s, 3H, OCH₃), 4.89 (d, J=16.4 Hz, 1H, NCHHPh), 5.48 (d, J=16.4 Hz, 1H, NCHHPh), 6.52 (d, J=1.6 Hz, 1H, ArH), 6.71 (d, J=1.6 Hz, 1H, ArH), 6.76-6.84 (m, 2H, ArH), 7.00-7.06 (m, 2H, ArH), 7.12-7.31 (m, 3H, ArH), 7.41-7.45 (m, 1H, ArH) ; m.p. 113-114° C.; HRMS m/e cacld. for C₂₈H₂₇N₃O₃ BrCl 567.0924, found 567.0933.

### EXAMPLE 34

### 1-(3-Bromo-benzyl)-7-chloro-3-(4-hydroxy-phenyl)-3-methyl-5-piperazin-1-yl-1H-quinoline-2,4-dione

The title compound was prepared according to the same procedure as for the example 2, using the example 33 (106 mg, 0.19 mmol) and BBr₃ (0.56 mmol, in 1M dichloromethane). After normal workup, the crude was purified by a flash column chromatography (CH₂Cl₂:MeOH=10:1) to provide the pure title compound (84 [symbol] 76%) as a pale yellow solid:

¹H NMR (200 MHz, CDCl₃+CD₃OD) δ 1.73 (s, 3H, CH₃), 2.82-2.87 (m, 2H, NCH₂), 3.00-3.18 (m, 6H, 3×NCH₂), 4.82 (d, J=16.2 Hz, 1H, NCHHPh), 5.46 (d, J=16.2 Hz, 1H, NCHHPh), 6.49 (d, J=1.6 Hz, 1H, ArH), 6.70 (d, J=1.6 Hz, 1H, ArH), 6.76-6.86 (m, 2H, ArH), 7.04-7.12 (m, 2H, ArH), 7.14-7.28 (m, 3H, ArH), 7.36-7.40 (m, 1H, ArH); decomp. 270° C.; MS(EI) m/e 553[M⁺]; HRMS m/e cacld. for C₂₇H₂₅N₃O₃ClBr 553.0768, found 553.0789.

### EXAMPLE 35

### 1-(2-Bromo-benzyl)-7-chloro-3-(4-methoxy-phenyl)-3-methyl-5-piperazin-1-yl-1H-quinoline-2,4-dione

The title compound was prepared according to the same procedure as for the example 5, using the intermediate III-16 (1.00 g, 1.92 mmol), piperazine (0.49 g, 5.77 mmol) and K₂CO₃(0.87 g, 80%). After normal workup, the crude was purified by a flash column chromatography (CH₂Cl₂:MeOH=10:1) to afford the pure title compound (0.15 g, 65%) as a pale yellow solid:

¹H NMR (200MHz, CDCl₃) δ1.78 (s, 3H, C_{H3}), 2.88-2.93 (m, 2H, NCH₂), 3.11-3.20 (m, 6H, 3×NCH₂), 3.76 (s, 3H, OCH₃), 5.03 (d, J=16.4 Hz, 1H, NCHHPh), 5.45 (d, J=16.4 Hz, 1H, NCHHPh), 6.41 (d, J=1.8 Hz, 1H, ArH), 6.71-6.81 (m, 4H, ArH), 7.03-7.17 (m, 4H, ArH), 7.60-7.64 (m, 1H, ArH); m.p. 150-151° C.; MS(EI) m/e 569[M⁺], 539, 527; HRMS m/e cacld. for C₂₈H₂₇N₃O₃ClBr 567.0924, found 567.0934.

### EXAMPLE 36

### 1-(2-Bromo-benzyl)-7-chloro-3-(4-hydroxy-phenyl)-3-methyl-5-piperazin-1-yl-1H-quinoline-2,4-dione

The title compound was prepared according to the same procedure as for the example 2, using the example 35 (1.00 g, 1.76 mmol) and BBr (5.27 mmol, in 1M dichloromethane). After normal workup, the crude was purified by a flash column chromatography (CH₂Cl₂:MeOH=5:1) to provide the pure title compound (0.75 g, 77%) as a pale yellow solid:

¹H NMR (200MHz, CD₃OD) δ1.72 (s, 3H, CH₃), 2.94-3.03 (m, 2H, NCH₂), 3.22-3.37 (m, 6H, 3×NCH₂), 5.08 (d, J=16.4 Hz, 1H, NCHHPh), 5.45 (d, J=16.4 Hz, 1H, NCHHPh), 6.52 (d, J=1.8 Hz, 1H, ArH), 6.68-6.73 (m, 2H, ArH), 6.80-7.00 (m, 4H, ArH), 7.18-7.27 (m, 2H, ArH), 7.65-7.69 (m, 1H, ArH); m.p 264-265° C.; MS(EI) m/e 553[M⁺], 525, 513; HRMS m/e cacld. for C₂₇H₂₅N₃O₃ClBr 553.0768, found 553.0746.

### EXAMPLE 37

### 7-Chloro-3-(4-methoxy-phenyl)-3-methyl-5-piperazin-1-yl-1-pyridin-3-ylmethyl-1H-quinoline-2,4-dione

The title compound was prepared according to the same procedure as for the example 1, using the intermediate III-17 (0.12 g, 0.27 mmol) and piperazine (0.06 g, 0.68 mmol). After normal workup, the crude was purified by a flash column chromatography (CH₂Cl₂:MeOH=10:1) to afford the pure title compound (0.03 g, 23%) as a yellow solid:

¹H NMR (200 MHz, CDCl₃) δ 1.76 (s, 3H, CH₃), 2.581 (br s, 1H, NH), 2.83-2.94 (m, 2H, NCH₂), 3.09-3.24 (m, 6H, 3×NCH₂), 3.74 (s, 3H, OCH₃), 5.12 (d, J=16.4 Hz, 1H, NCHHPh), 5.37-5.45 (d, J=16.4 Hz, 1H, NCHHPh), 6.56 (d, J=1.6 Hz, 1H, ArH), 6.68-6.69 (d, J=1.6 Hz, 1H, ArH), 6.73-6.77 (m, 2H, ArH), 6.94-7.00 (m, 2H, ArH), 7.21-7.28 (m, 1H, ArH), 7.47-7.51 (m, 1H, ArH), 8.53-8.56 (m, 2H, ArH); m.p. 160-162° C.; MS(EI) m/e 490[M⁺], 460, 448; HRMS m/e cacld. for C₂₇H₂₇ N₄O₃Cl 490.1772, found 490.1779.

### EXAMPLE 38

### 7-Chloro-3-(4-hydroxy-phenyl)-3-methyl-5-piperazin-1-yl-1-pyridin-3-ylmethyl-1H-quinoline-2,4-dione

The title compound was prepared according to the same procedure as for the example 2, using the example 37 (30 mg, 0.061 mmol) and BBr (0.18 mmol, in 1M dichloromethane). After normal workup, the crude was purified by a flash column chromatography (CH₂Cl₂:MeOH=5:1) to provide the pure title compound (12 mg, 41%) as a pale yellow solid:

¹H NMR(200 MHz, CDCl₃+CD₃OD) δ 1.74 (s, 3H, CH₃), 2.90-2.97 (m, 3H, NCH₂, NCHH), 3.16-3.24 (m, 5H, 2×NCH₂, NCHH), 4.97 (d, J=16.2 Hz, 1H, NCH HPh), 5.48 (d, J=16.2 Hz, 1H, NCHHPh), 6.56 (d, J=2.4 Hz, 1H, ArH), 6.66-6.71 (m, 2H, ArH), 6.74 (d, J=2.4 Hz, 1H, ArH), 7.31-7.37 (m, 1H, ArH), 7.58-7.62 (m, 1H, ArH), 8.42 (m, 1H, ArH), 8.49-8.52 (m, 1H, ArH); decomp. 270° C.; MS(EI) m/e 476[M⁺], 446, 434; HRMS m/e cacld. for C₂₆H₂₅N₄O₃Cl 476.1615, found 476.1615.

### EXAMPLE 39

### 7-Chloro-3-(4-methoxy-phenyl)-3-methyl-1-naphthalen-2-ylmethyl-5-piperazin-1-yl-1H-quinoline-2,4-dione

The title compound was prepared according to the same procedure as for the example 5, using the intermediate III-18 (0.1 g, 0.20 mmol), piperazine (44 [symbol] 0.51 mmol) and triethylamine (0.15 [symbol] 1.00 mmol) as a base. After normal workup, the crude was purified by a flash column chromatography (CH₂Cl₂:MeOH=10:1) to afford the pure title compound (67 mg, 61%) as a yellow solid:

¹H NMR (200 MHz,CDCl₃) δ 1.79 (s, 3H, CH₃), 2.86-2.90 (m, 2H, NCH₂), 3.08-3.17 (m, 6H, NCH×3), 3.76 (s, 3H, OCH₃), 5.08 (d, J=16.8 Hz, 1H, NCHHPh), 5.63 (d, J=16.8 Hz, 1H, NCHHPh), 6.64 (d, J=1.8 Hz, 1H, ArH), 6.67 (d, J=1.8 Hz, 1H, ArH), 6.75-6.80 (m, 2H, ArH), 7.03-7.09 (m, 2H, ArH), 7.33 (dd, J=8.6, 1.6 Hz, ArH), 7.43-7.50 (m, 3H, ArH), 7.63-7.68 (m, 1H, ArH), 7.81-7.85 (m, 2H, ArH); m.p. 169-170° C.; MS(EI) m/e 539[M⁺], 509, 497; HRMS m/e cacld. for C₃₂H₃₀ N₃O₃Cl 539.1976, found 539.1957.

### EXAMPLE 40

### 7-Chloro-3-(4-hydroxy-phenyl)-3-methyl-1-naphthalen-2-ylmethyl-5-piperazin-1-yl-1H-quinoline-2,4-dione

The title compound was prepared according to the same procedure as for the example 2, using the example 39 (50 mg, 0.094 mmol) and BBr (0.28 mmol, in 1M dichloromethane). After normal workup, the crude was purified by a flash column chromatography (CH₂Cl₂:MeOH=10:1) to provide the pure title compound (30 [symbol] 61%) as a pale yellow solid:

¹H NMR (200 MHz, CDCl₃+CD₃OD) δ 1.78 (s, 3H, CH₃), 2.86-2.98 (m, 2H, NCH₂), 3.10-3.27 (m, 6H, 3×NCH₂), 5.11 (d, J=16.8 Hz, 1H, NCHHPh), 5.63 (d, J=16.8 Hz, 1H, NCHHPh), 6.70-6.76 (m, 4H, ArH), 6.94-6.99 (m, 2H, ArH), 7.34-7.39 (m, 1H, ArH), 7.43-7.51 (m, 3H, ArH), 7.66-7.71 (m, 1H, ArH), 7.81-7.86 (m, 2H, ArH); decomp. 265° C.; HRMS m/e cacld. for C₃₁H₂₈N₃O₃Cl 525.1819, found 525.1794.

### EXAMPLE 41

### 1-Biphenyl-4-ylmethyl-7-chloro-3-(4-methoxy-phenyl)-3-methyl-5-piperazin-1-yl-1H-quinoline-2,4-dione

The title compound was prepared according to the same procedure as for the example 9, using the intermediate III-19 (0.23 g, 0.45 mmol), piperazine (0.1 g, 1.1 mmol) in pyridine (5 [symbol] ). After normal workup, the crude was purified by a flash column chromatography (CH₂Cl₂:MeOH=10:1) to afford the pure title compound (0.15 g, 60%) as a yellow solid:

¹H NMR (200 MHz, CDCl₃) δ 1.78 (s, 3H, CH₃), 2.90-2.93 (m, 3H, NCH₂, NCH H), 3.13-3.22 (m, 5H, 2×NCH₂, NCHH, NH), 3.74 (s, 3H), 5.06 (d, J=16.0 Hz, 1H, NCHHPh), 5.44 (d, J=16.0 Hz, 1H, NCHHPh), 6.68 (m, 2H, ArH), 6.73-6.78 (m, 2H, ArH), 7.01-7.06 (m, 2H, ArH), 7.25-7.29 (m, 2H, ArH), 7.34-7.59 (m, 7H, ArH); m.p. 142-144° C.; MS(EI) m/e 565[M⁺], 548, 523; HRMS m/e cacld. for C₃₄H₃₂ N₃O₃Cl 565.2132, found 565.2136.

### EXAMPLE 42

### 1-Biphenyl-4-ylmethyl-7-chloro-3-(4-hydroxy-phenyl)-3-methyl-5-piperazin-1-yl-1H-quinoline-2,4-dione

The title compound was prepared according to the same procedure as for the example 2, using the example 41 (70 [symbol] 0.12 mmol) and BBr(0.37 mmol, in 1M dichloromethane). After normal workup, the crude was purified by a flash column chromatography (CH₂Cl₂:MeOH=5:1) to provide the pure title compound (35 [symbol] 53%) as a pale yellow solid:

¹H NMR (200 MHz, CDCl₃+CD₃OD) δ 1.78 (s, 3H, CH₃), 2.86-2.95 (m, 2H, NCH₂), 3.12-3.21 (m, 5H, 2×NCH₂, NCHH), 3.35-3.38 (m, 1H, NCHH), 5.12 (d, J=16.2 Hz, 1H, NCHHPh), 5.43 (d, J=16.2 Hz, 1H, NCHHPh), 6.69-6.73 (m, 2H, ArH) 6.92-6.98(m, 2H, ArH), 7.26-7.30 (m, 2H, ArH), 7.34-7.48 (m, 4H, ArH), 7.55-7.61 (m, 5H, ArH); m.p. 240-241° C.; MS(EI) m/e 551[M⁺], 521, 509; HRMS m/e cacld. for C₃₃H₃₀N₃O₃Cl 551.1976, found 551.1963.

### EXAMPLE 43

### 1-(1H-Benzoimidazol-2-ylmethyl)-7-chloro-3-(4-methoxy-phenyl)-3-methyl-5-piperazin-1-yl-1H-quinoline-2,4-dione

The title compound was prepared according to the same procedure as for the example 5, using the intermediate III-20 (1.00 g, 2.08 mmol), piperazine (0.54 g, 6.24 mmol) and K₂CO₃ (0.86 g, 6.24 mmol). After normal workup, the crude was purified by a flash column chromatography (CH₂Cl₂:MeOH=10:1) to afford the pure title compound (0.80 g, 73%) as a pale yellow solid:

¹H NMR (200 MHz, CD₃OD) δ1.78 (s, 3H, CH₃), 2.83-2.89 (m, 2H, NCH₂), 3.02-3.15 (m, 6H, 3×NCH₂), 3.72 (s, 3H, OCH₃), 5.27 (d, J=16.4 Hz, 1H, NCHHPh), 5.87 (d, J=16.4 Hz, 1H, NCHHPh), 6.77-6.83 (m, 3H, ArH), 6.98-7.09 (m, 3H, ArH), 7.22-7.27 (m, 2H, ArH), 7.50-7.58 (m, 2H, ArH); m.p 178-179° C.; MS(EI) m/e 529[M⁺], 495, 465, 439; HRMS m/e cacld. for C₂₉H₂₈N₅O₃Cl 529.1881, found 529.1875.

### EXAMPLE 44

### 7-Chloro-3-(4-methoxy-phenyl)-3-methyl-5-piperazin-1-yl-1-quinolin-2-ylmethyl-1H-quinoline-2,4-dione

The title compound was prepared according to the same procedure as for the example 5, using the intermediate III-21 (1.00 g, 2.03 mmol), piperazine (0.52 g, 6.10 mmol) and K₂CO₃ (0.84 g, 6.10 mmol). After normal workup, the crude was purified by a flash column chromatography (CH₂Cl₂:MeOH=10:1) to afford the pure title compound (0.87 g, 79%) as a pale yellow solid:

¹H NMR (200 MHz,CDCl₃) δ 1.85 (s, 3H, CH₃), 2.78-2.86 (m, 2H, NCH₂), 2.99-3.07 (m, 6H, 3×NCH₂), 3.77 (s, 3H, OCH₃), 5.21 (d, J=16.4 Hz, 1H, NCHHPh), 5.82 (d, J=16.4 Hz, 1H, NCHHPh), 6.63 (d, J=1.8 Hz, 1H, ArH), 6.77-6.83 (m, 2H, ArH), 6.94 (d, J=1.8 Hz, 1H, ArH), 7.23-7.29 (m, 2H, ArH), 7.38 (d, J=8.4 Hz, 1H, ArH), 7.52-7.60 (m, 1H, ArH), 7.70-7.85 (m, 2H, ArH), 8.06-8.18 (m, 2H, ArH); m.p 200-201° C.; MS(EI) m/e 540[M⁺], 523, 510, 498, 484, 464; HRMS m/e cacld. for C₃₁H₂₉N₄O₃Cl 1 540.1928, found 540.1930.

### EXAMPLE 45

### 7-Chloro-3-(4-hydroxy-phenyl)-3-methyl-5-piperazin-1-yl-1-quinolin-2-ylmethyl-1H-quinoline-2,4-dione

The title compound was prepared according to the same procedure as for the example 2, using the example 44 (1.00 g, 1.85 mmol) and BBr (5.54 mmol, in 1M dichloromethane). After normal workup, the crude was purified by a flash column chromatography (CH₂Cl₂:MeOH=5:1) to provide the pure title compound (0.83 g, 85%) as a pale yellow solid:

¹H NMR (200 MHz, CD₃OD) δ 1.78 (s, 3H, CH₃), 2.83-2.92 (m, 2H, NCH₂), 3.11-3.19 (m, 6H, 3×NCH₂), 5.37 (d, J=17.2 Hz, 1H, NCHHPh), 5.74 (d, J=17.2 Hz, 1H, NCHHPh), 6.69-6.78 (m, 2H, ArH), 6.87 (dd, J=9.5, 1.8 Hz, 1H, ArH), 7.04 (d, J=1.8 Hz, 1H, ArH), 7.20-7.26 (m, 2H, ArH), 7.47-7.63 (m, 2H, ArH), 7.72 (td, J=7.3, 1.8 Hz, 1H, ArH), 7.95 (t, J=8.4 Hz, 2H, ArH), 7.38 (d, J=8.4 Hz, 1H, ArH); m.p 278-279° C.; MS(EI) m/e 526[M⁺], 510, 497.

### EXAMPLE 46

### 7-Chloro-1-ethyl-3-(4-methoxy-phenyl)-3-methyl-5-piperazin-1-yl-1H-quinoline-2,4-dione

The title compound was prepared according to the same procedure as for the example 5, using the intermediate III-22 (0.14 g, 0.37 mmol), piperazine (80 [symbol] 0.93 mmol) and triethylamine (0.3 [symbol] 1.9 mmol) as a base. After normal workup, the crude was purified by a flash column chromatography (CH₂Cl₂:MeOH=10:1) to afford the pure title compound (90 [symbol] 56%) as a pale yellow solid:

¹H NMR (200 MHz,CDCl₃) δ 1.32 (t, J=6.8 Hz, 3H, CH₂CH₃), 1.73 (s, 3H, CH₃), 2.22 (br s, 1H, NH), 2.80-2.88 (m, 2H, NCH₂), 3.04-3.11 (m, 6H, 3×NCH₂), 3.73 (s, 3H, OCH₃), 3.90-4.00 (m, 1H, NCHHMe), 4.21-4.31 (m, 1H, NCHHMe), 6.65-6.68 (m, 2H, ArH), 6.72-6.77 (m, 2H, ArH), 6.98-7.02 (m, 2H, ArH); m.p. 127-129° C.; MS(EI) m/e 427[M⁺], 397, 385; HRMS m/e cacld. for C₂₃H₂₆N₃O₃Cl 427.1663, found 427.1667.

### EXAMPLE 47

### 5-Chloro-1-ethyl-3-(4-hydroxy-phenyl)-3-methyl-7-piperazin-1-yl-1H-quinoline-2,4-dione

The title compound was prepared according to the same procedure as for the example 2, using the example 46 (0.09 g, 0.25 mmol) and BBr₃ (0.74 mmol, in 1M dichloromethane). After normal workup, the crude was purified by a flash column chromatography (CH₂Cl₂:MeOH=5:1) to provide the pure title compound (0.06 g, 68%) as a pale yellow solid:

¹H NMR (200 MHz, CDCl₃+CD₃OD) δ 1.14 (t, 3H, J=6.8 Hz, CH₂CH₃), 1.50 (s, 3H, CH₃), 2.68-2.79 (m, 2H, NCH₂), 2.99-3.10 (m, 6H, 3×NCH₂), 3.71-3.82 (m, 1H, NCHHCH₃), 6.47-6.60 (m, 4H, ArH), 6.67-6.71 (m, 2H, ArH); m.p. 290-292° C.; MS(EI) m/e 413[M⁺], 383, 371 HRMS m/e cacld. for C₂₂H₂₄N₃O₃Cl 413.1506, found 413.1507.

### EXAMPLE 48

### 7-Chloro-1-cyclohexylmethyl-3-(4-methoxy-phenyl)-3-methyl-5-piperazin-1-yl-1H-quinoline-2,4-dione

The title compound was prepared according to the same procedure as for the example 5, using the intermediate III-23 (0.18 g, 0.40 mmol), piperazine (90 mg, 1.00 mmol) and triethylamine (0.30 [symbol] 2.00 mmol) as a base. After normal workup, the crude was purified by a flash column chromatography (CH₂Cl₂:MeOH=10:1) to afford the pure title compound (0.13 g, 65%) as a pale yellow solid:

¹H NMR (200 MHz,CDCl₃) δ 1.01-1.25 (m, 5H, cyclohexyl), 1.53-1.71 (m, 5H, CH₃, cyclohexyl), 2.03-2.09 (m, 4H, cyclohexyl), 2.83-2.89 (m, 2H, NCH₂), 3.08-3.10 (m, 6H, 3×NCH₂), 3.67-3.77 (m, 4H, OCH₃ & NCHH-cyclohexyl), 4.06-4.18 (m, 1H, NCHH-cyclohexyl), 6.66 (br s, 2H, ArH), 6.70-6.75 (m, 2H, ArH), 6.96-7.00 (m, 2H, ArH); m.p. 127-128° C.; MS(EI) m/e 495[M+], 465, 453; HRMS m/e cacld. for C₂₈H₄₃N₃O₃Cl 495.2289, found 495.2284.

### EXAMPLE 49

### 7-Chloro-1-cyclohexylmethyl-3-(4-hydroxy-phenyl)-3-methyl-5-piperazin-1-yl-1H-quinoline-2,4-dione

The title compound was prepared according to the same procedure as for the example 2, using the example 48 (128 [symbol] 0.26 mmol) and BBr(0.77 mmol, in 1M dichloromethane). After normal workup, the crude was purified by a flash column chromatography (CH₂Cl₂:MeOH=10:1) to provide the pure title compound (98 [symbol] 75%) as a pale yellow solid:

¹H NMR (200 MHz, CDCl₃+CD₃OD) δ 1.07-1.26 (m, 6H, cyclohexyl), 1.58-1.70 (m, 8H, cyclohexyl, CH₃), 2.85-2.93 (m, 2H, NCH₂), 3.13-3.20 (m, 6H, 3×NCH₂), 3.67-3.77 (m, 1H, NCHH-cyclohexyl), 4.08-4.19 (m, 1H, NCHH-cyclohexyl), 6.63-6.71 (m, 4H, ArH), 6.85-6.90 (m, 2H, ArH); m.p. 175-176° C.; MS(EI) m/e 481 [M⁺], 451, 439; HRMS m/e cacld. for C₂₇H₃₂N₃O₃Cl 481.2232, found 481.2137.

### EXAMPLE 50

### 1-Benzyl-7-chloro-3-(3-methoxy-phenyl)-3-methyl-5-piperazin-1-yl-1H-quinoline-2,4-dione

The title compound was prepared according to the same procedure as for the example 5, using the intermediate III-24 (0.47 g, 1.10 mmol), piperazine (0.28 g, 3.22 mmol) and K₂CO ₃(0.76 g, 5.50 mmol). After normal workup, the crude was purified by a flash column chromatography (CH₂ Cl₂:MeOH=10:1) to afford the pure title compound (0.39 g, 72%) as a pale yellow solid:

1H NMR (200 MHz, CDCl₃) δ 1.79 (s, 3H, CH₃), 2.80-2.90 (m, 2H, NCH₂), 2.99-3.17 (m, 6H, 3×NCH₂), 3.68 (s, 3H, OCH₃), 5.02 (d, J=17.4 Hz, 1H, NCHHPh), 5.41 (d, J=17.4 Hz, 1H, NCHHPh), 6.61 (d, J=1.4 Hz, 1H, ArH), 6.66-6.68 (m, 3H, ArH), 6.72-6.78 (m, 2H, ArH), 7.11-7.33 (m, 6H, ArH); m.p. 110-112° C.; MS(EI) m/e 489[M⁺], 459, 447; HRMS m/e cacld. for C₂₈H₂₈N₃₃Cl 489.1819, found 48 9.1831.

### EXAMPLE 51

### 1-Benzyl-7-chloro-3-(3-hydroxy-phenyl)-3-methyl-5-piperazin-1-yl-1H-quinoline-2,4-dione

The title compound was prepared according to the same procedure as for the example 2, using the example 50 (0.27 g, 0.55 mmol) and BBr (1.60 mmol, in 1M dichloromethane). After normal workup, the crude was purified by a flash column chromatography (CH₂Cl₂:MeOH=5:1) to provide the pure title compound (0.20 g, 77%), as a pale yellow solid:

¹H NMR (200 MHz, CDCl₃) δ 1.79 (s, 3H, CH₃), 2.80-2.84 (m, 2H, NCH₂), 3.02 (m, 6H, 3×NCH₂), 5.08 (d, J=16.2 Hz, 1H, NCHHPh), 5.39 (d, J=16.2 Hz, 1H, NCHHPh), 6.60-6.69 (m, 4H, ArH), 7.04-7.12 (dd, J=8.2 Hz, 8.1 Hz, 1H, ArH), 7.21-7.35 (m, 6H, ArH); m.p. 238-240° C.; MS(EI) m/e 475[M⁺], 91, 56; HRMS m/e cacld. for C₂₇H₂₆N₃O₃Cl 475.1663, found 475.1665.

Structures of compounds prepared by the above examples are listed in Table 1.

TABLE 1

| Ex- | |
|---|---|
| ample | Formula |
| 1 | 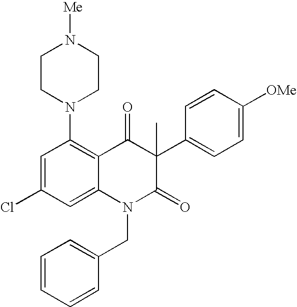 |
| 2 | 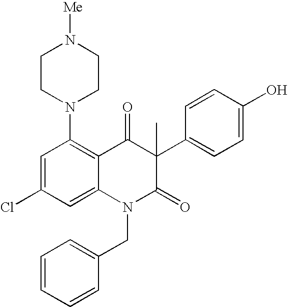 |
| 3 | 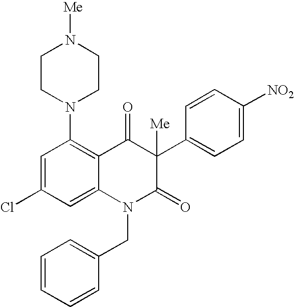 |
| 4 | 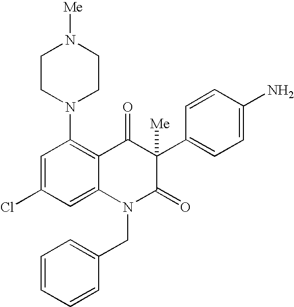 |
| 5 | 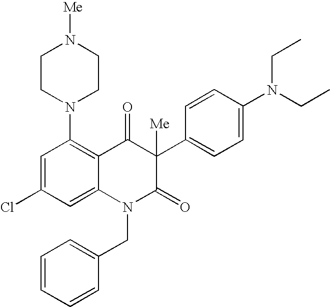 |
| 6 | 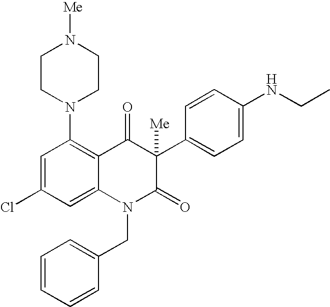 |
| 7 | 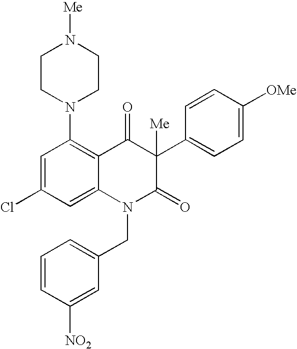 |
| 8 | 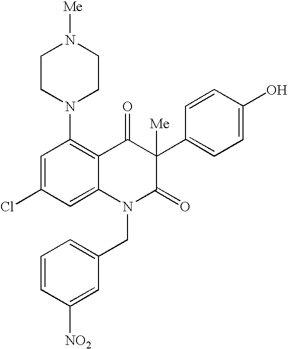 |
| 9 | 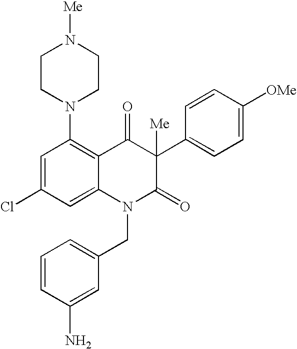 |
| 10 | 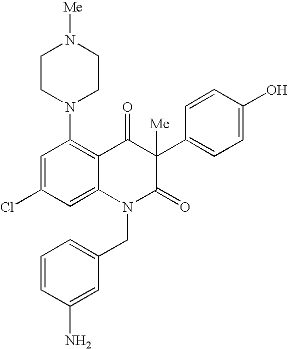 |
| 11 | 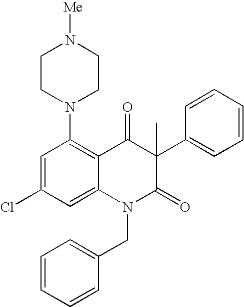 |
| 12 | 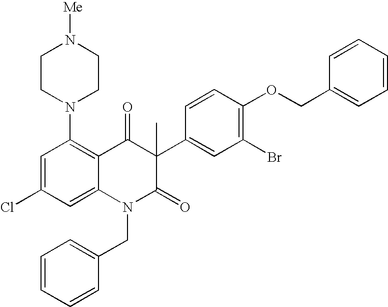 |
| 13 | 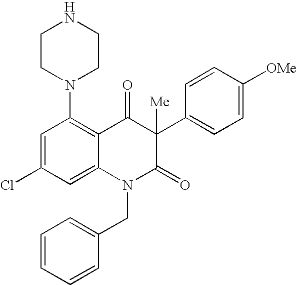 |
| 13-1 | 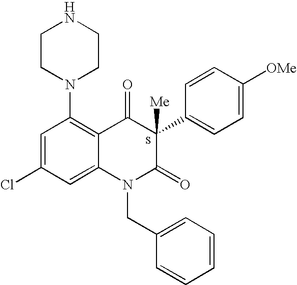 |
| 13-2 | 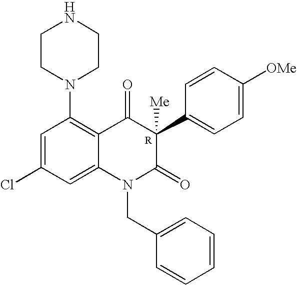 |
| 14 | 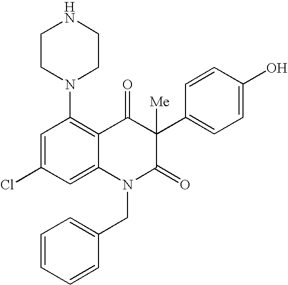 |
| 14-1 | 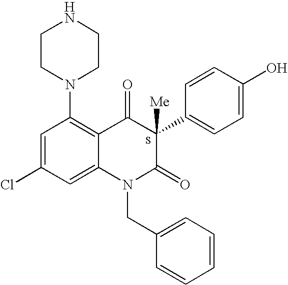 |
| 14-2 | 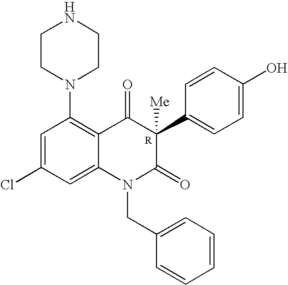 |
| 15 | 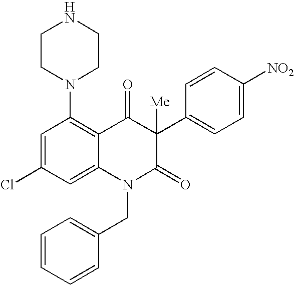 |
| 16 | 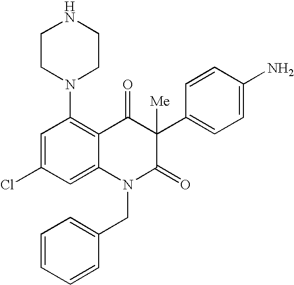 |
| 17 | 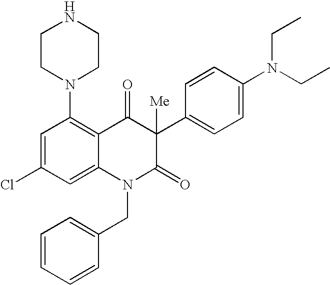 |
| 18 | 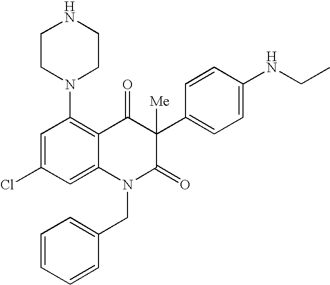 |
| 19 | 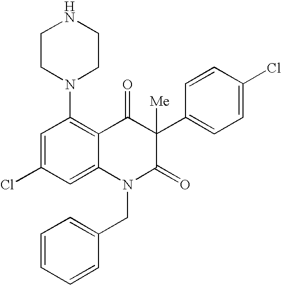 |
| 20 | 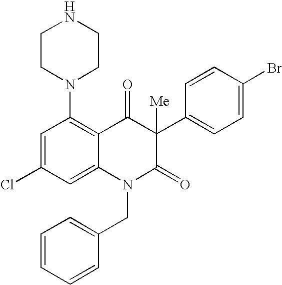 |
| 21 | 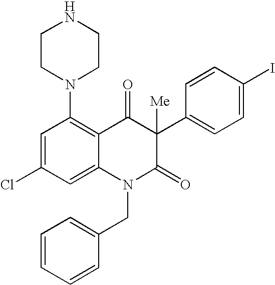 |
| 22 | 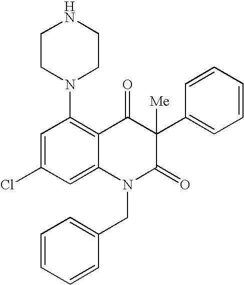 |
| 23 | 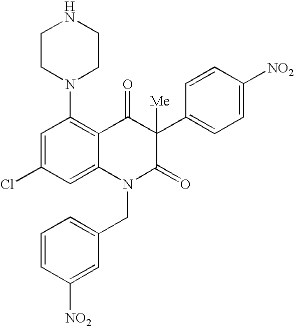 |
| 24 | 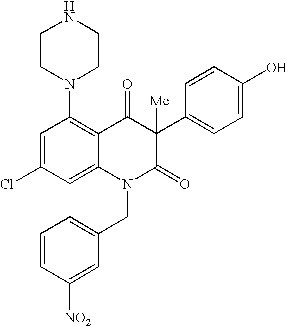 |
| 25 | 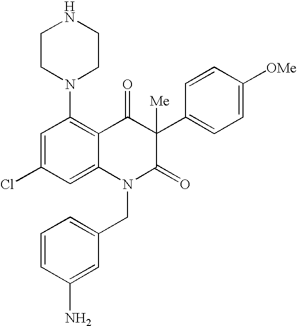 |
| 26 | 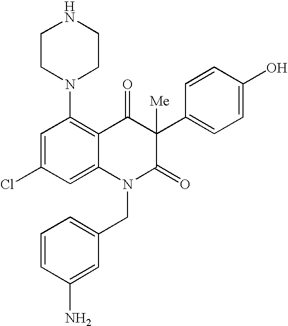 |
| 27 | 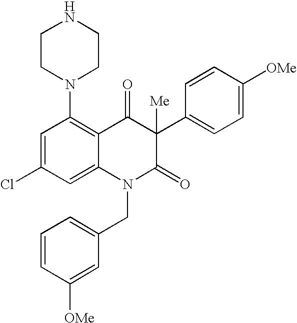 |
| 28 | 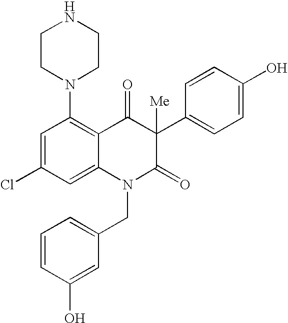 |
| 29 | 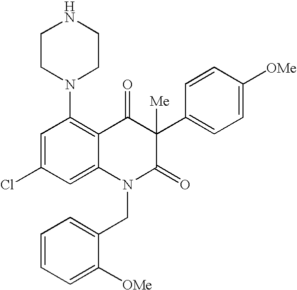 |
| 30 | 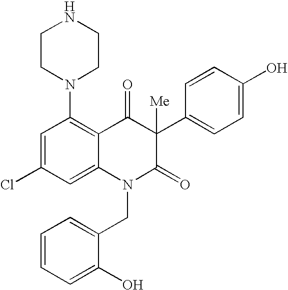 |
| 31 | 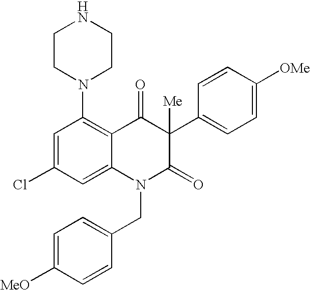 |
| 32 | 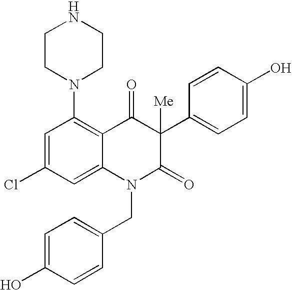 |
| 33 | 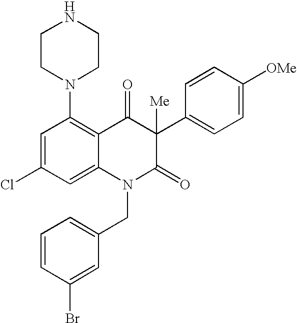 |
| 34 | 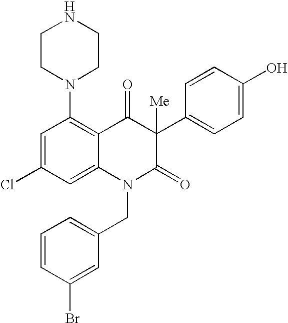 |
| 35 | 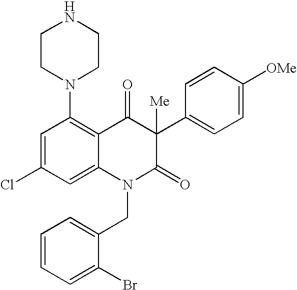 |
| 36 | 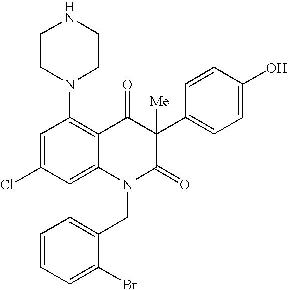 |
| 37 | 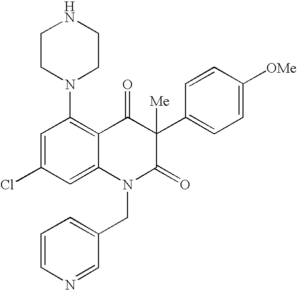 |
| 38 | 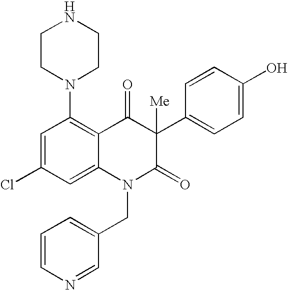 |
| 39 | 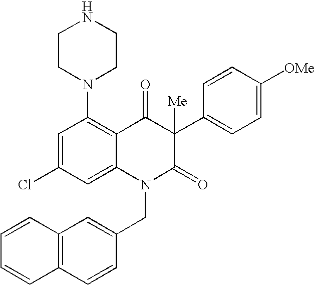 |
| 40 | 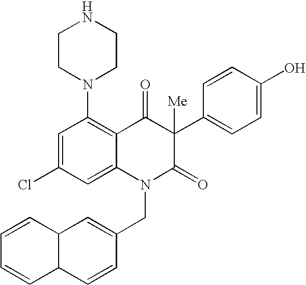 |
| 41 | 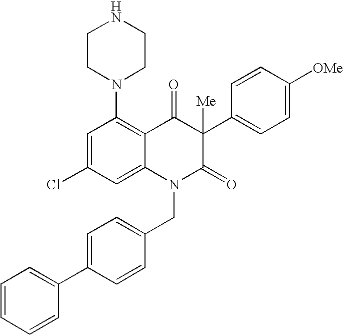 |
| 42 | 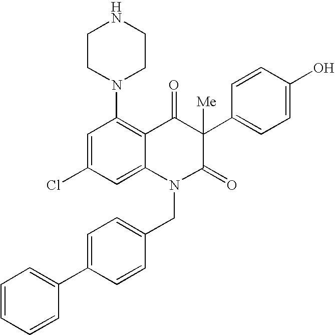 |
| 43 | 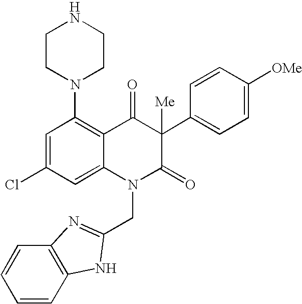 |
| 44 | 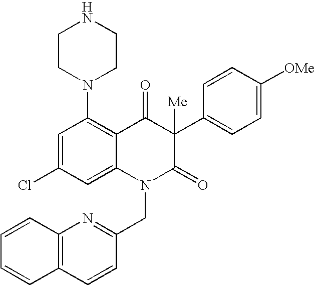 |
| 45 | 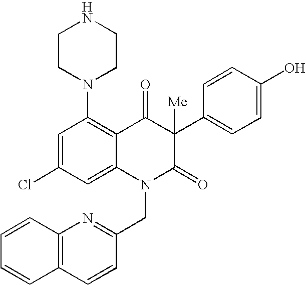 |
| 46 | 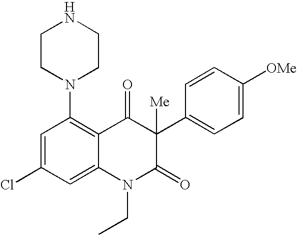 |
| 47 | 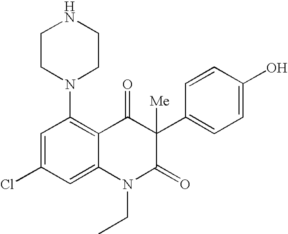 |
| 48 | 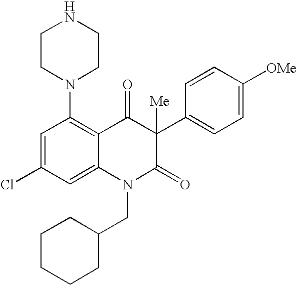 |
| 49 | 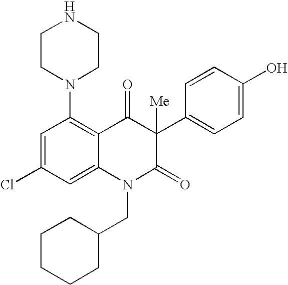 |
| 50 | 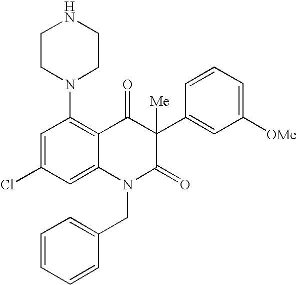 |
| 51 | 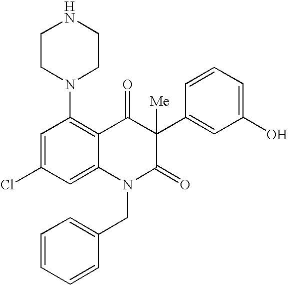 |

### EXPERIMENTAL EXAMPLE 1

### Binding Affinity of the Compounds According to the Present Invention to 5-HT6 Receptors

1-1: Expression of Human Serotonin 5-HT6 Receptor

Human serotonin 5-HT6 receptor protein was expressed in insect cell as described below. Human 5-HT6 cDNA was cloned from human brain cDNA library (Clontech, Palo Alto, USA) by PCR amplification using 5′-TCATCTGCTTTCCCGCCACCCTAT-3′ for forward and 5′-TCAGGGTCTGGGTTCTGCTCAATC-3′ for reverse. Amplified cDNA fragments were introduced into pGEMT easy vector (Promega, Madison, USA) and then DNA sequencing was performed to confirm receptor DNA sequence. Serotonin 5-HT6 clone was subcloned into insect cell expression vector BacPAK8 (Clontech). pBacPAK8/5-HT6 was transfected into insect Sf21 cell (Clontech) and protein expression of 5-HT6 receptor was confirmed by SDS PAGE and receptor binding assay. Cell lysis was performed by sonication for 2 minutes at 4° C. and cell debris was discarded by centrifugation for 10 min at 3,000×g. Membrane fraction was purified partially from supernatant above by centrifugation for 1 hr at 100,000×g.

1-2: Measurement of Binding Affinity to the Cloned 5-HT6 Receptors

The binding affinity of the compound according to the present invention to 5-HT6 receptor using the cloned 5-HT6 receptor as following.

[³H]LSD(lysergic acid diethylamide) binding assay was performed in 96-well plate to test the binding affinities of the compounds according to the present invention on 5-HT6 receptor. The cloned receptor membranes (9 [symbol] well) were used in a final volume of 0.25 [symbol] reaction mixture and incubated at 37° C. for 60 min with 50 mM Tris-HCl buffer (pH 7.4) involving 10 mM MgCl₂ and 0.5 mM EDTA. For drug screening, testing compounds were incubated as described above, in a reaction mixture containing 1.87 nM of [³H]LSD. After incubation, the reaction was terminated by the rapid filtration and washed with ice-cold 50 mM Tris-HCl buffer using a Inotech harvester (Inotech, Switzerland) through Wallac GF/C glass fiber filter (Wallac, Finland) which was presoaked in 0.5% PEI. The filter was covered with MeltiLex, sealed in a sample bag followed by drying in the oven, and counted by MicroBeta Plus (Wallac, Finland). Competition binding studies were carried out with 7-8 concentrations of the compound according to the present invention run in duplicate tubes, and isotherms from three assays were calculated by computerized nonlinear regression analysis (GraphPad Prism Program, San Diego, Canada) to yield median inhibitory concentration (IC₅₀) values. Non-specific binding was determined in the presence of 10 μM methiothepin. All testing compounds were dissolved in dimethylsulfoxide (DMSO), and serially diluted to various concentrations for binding assays. 5-HT6 receptor binding affinities of the the compounds according to the present invention were shown in Table 2.

TABLE 2
Binding affinity of the compounds according to the
present invention to the 5-HT6 receptor

| Example | IC₅₀ (nM) |
|---|---|
| 1 | 1.9 |
| 2 | 2.5 |
| 3 | 4.7 |
| 4 | 7.4 |
| 5 | 37.8 |
| 6 | 3.4 |
| 7 | 16.6 |
| 8 | 23.8 |
| 9 | 5.2 |
| 10 | 6.2 |
| 11 | 1.4 |
| 12 | 9.2 |
| 13 | 0.9 |
| 13-1 | 1.4 |
| 13-2 | 9.0 |
| 14 | 1.0 |
| 14-1 | 1.9 |
| 14-2 | 13.3 |
| 15 | 12.1 |
| 16 | 3.2 |
| 17 | 41.5 |
| 18 | 14.6 |
| 19 | 2.3 |
| 20 | 1.9 |
| 21 | 6.9 |
| 22 | 2.7 |
| 23 | 2.7 |
| 24 | 14.5 |
| 25 | 6.2 |
| 26 | 8.7 |
| 27 | 2.3 |
| 28 | 3.0 |
| 29 | 34.2 |
| 30 | 15.9 |
| 31 | 26.5 |
| 32 | 1.7 |
| 33 | 1.8 |
| 34 | 3.6 |
| 35 | 9.2 |
| 36 | 29.0 |
| 37 | 21.4 |
| 38 | 30.6 |
| 39 | 4.7 |
| 40 | 55.7 |
| 41 | 351.2 |
| 42 | 163.7 |
| 43 | 136.7 |
| 44 | 6.9 |
| 45 | 11.4 |
| 46 | 49.1 |
| 47 | 151.4 |
| 48 | 250.9 |
| 49 | 11.7 |
| 50 | 1.8 |
| 51 | 6.2 |

As shown in the Table 2, the compounds prepared by Example 1 to 51 of the present invention had good binding affinities at 5-HT6 receptor labeled by [³H]LSD, and particularly the compounds of Example 1, 11, 13, 13-1, 14, 14-1, 20, 32, 33 and 50 showed significant affinities.

### EXPERIMENTAL EXAMPLE 2

### Radioligand Binding Studies for 5-HT6 Receptor Selectivity

The following tests were performed to survey how much the compound showing excellent affinity to 5-HT6 receptor in the above experimental example 1 has selectivity for 5-HT6 receptor, compared to other 5-HT receptors and dopamine receptors.

2-1: Binding assays of 5-HT Receptor Family

Radioligand bindings were performed according to the test method provided by the supplier of receptor membrane (Euroscreen/BioSignal Packard Inc.). The detailed assay conditions and the results were shown in the following Table 3 and Table 4, respectively.

TABLE 3
Assay condition

| | 5-HT1a | 5-HT2a | 5-HT2c | 5-HT7 |
|---|---|---|---|---|
| Origin | Stable CHO-K1 cell strain expressing human | | | |
| | recombinant receptors (Euroscreen/BioSignal) | | | |
| Binding buffer | 50 mM Tris- | 50 mM Tris- | 50 mM Tris- | 50 mM Tris- |
| solution | HCl(pH 7.4) 10 mM | HCl(pH 7.4) | HCl(pH 7.7) 0.1% | HCl(pH 7.4) 10 mM |
| | MgSO₄ 0.5 mM | | ascorbic acid 10 μM | MgSO₄ 0.5 mM |
| | EDTA 0.1% | | Pargyline | EDTA |
| | ascorbic acid | | | |
| Final volume | 250 [symbol] | 250 [symbol] | 250 [symbol] | 250 [symbol] |
| Membrane | 40 [symbol] | 15 [symbol] | 4 [symbol] | 10 [symbol] |
| content | | | | |
| Radioligand | [³H]8-OH-DPAT | [³H]Ketanserin | [³H]Mesulergine 1 nM | [³H] LSD 3 nM |
| | 0.5 nM | 1 nM | | |
| Non-specific | methiothepin | Mianserin 1 M | methiothepin 10 M | methiothepin 10 M |
| Binding | 0.5 M | | | |
| Incubation | 27° C., 60 min | 37° C., 15 min | 37° C., 30 min | 27° C., 120 min |
| Filtration | GF/C, | GF/C, 0.05% | GF/C, 1% BSA | GF/C, 0.3% |
| | 0.3% PEI | Brij | | PEI |

2-2: Binding assays of Dopamine Receptor Family

The radioligands used were [³H] spiperone (for hD_{2L} and hD₃ receptors, 1 nM) and [³H] YM-09151-2 (for hD_{4.2} receptor, 0.06 nM). Radioligand bindings were performed by the protocols provided by the supplier of receptor membranes (BioSignal Packard Inc., Montreal, Canada). Briefly, the buffer used in D₂ or D₃ receptor binding assay was 50 mM Tris-HCl (pH 7.4), 10 mM MgCl₂, 1 mM EDTA, or 50 mM Tris-HCl (pH 7.4), 5 mM MgCl₂, 5 mM EDTA, 5 mM KCl, 1.5 mM CaCl₂, 120 mM NaCl, respectively. In [³H] YM-09151-2 receptor binding assays, the buffer containing 50 mM Tris-HCl (pH 7.4), 5 mM MgCl₂, 5 mM EDTA, 5 mM KCl and 1.5 mM CaCl₂ was used. Nonspecific binding was determined with haloperidol (10 μM) or clozapine (10 μM) for D₂ and D₃, and D₄ receptors, respectively. Competition binding studies were carried out with 7-8 concentrations of the test compound run in duplicate tubes, and isotherms from three assays were calculated by computerized nonlinear regression analysis (GraphPad Prism Program, San Diego, Canada) to yield median inhibitory concentration (IC₅₀) values.

The other serotonin receptor subtypes and dopamine selectivity of compounds according to the present invention was shown in Table 4.

TABLE 4
The other serotonin receptor subtypes and dopamine selectivity of compounds
according to the present invention

| | Binding affinity, IC₅₀ (nM) | | | | | | | | |
|---|---|---|---|---|---|---|---|---|---|
| Example | 5-HT6 | 5-HT7 | 5-HT1 a | 5-HT2 a | 5-HT2 c | D₁ | D₂ | D₃ | D₄ |
| 1 | 1.9 | 7486 | 4411 | 1120 | >10000 | 174 | >10000 | 2277 | 8017 |
| 2 | 2.5 | 920 | >10000 | 2621 | 487 | 4971 | >10000 | 589 | >10000 |
| 3 | 4.7 | 4568 | 5728 | 1544 | >10000 | 814 | >10000 | 360 | >10000 |
| 4 | 15.0 | 5046 | 5801 | 7011 | 5780 | 7089 | 6546 | 8115 | 8190 |
| 5 | 16.8 | 7615 | 6451 | 7540 | 4657 | 7701 | 7054 | 7084 | 8745 |
| 6 | 3.4 | >1000 | >10000 | >1000 | >1000 | >1000 | >1000 | 981 | >10000 |
| 7 | 20.0 | 8456 | 6554 | 6538 | 9405 | 8456 | 7148 | 8967 | 9087 |
| 8 | 16.8 | 9445 | 7513 | 6947 | 8812 | 7040 | 7289 | 9015 | 9154 |
| 9 | 5.2 | >10000 | >10000 | >10000 | >10000 | >10000 | >10000 | 821 | >10000 |
| 10 | 6.2 | >10000 | >10000 | >10000 | >10000 | >10000 | >10000 | 5374 | >10000 |
| 11 | 1.4 | 2205 | 2594 | 1232 | 514 | 4668 | 844 | 678 | >10000 |
| 12 | 9.2 | 136 | 2516 | 95 | 1287 | 1239 | 6446 | 153 | >10000 |
| 13 | 0.9 | 9003 | 1363 | 543 | 437 | 3586 | >10000 | 345 | >10000 |
| 13-1 | 1.4 | 5845 | 5433 | 2505 | 7742 | 6153 | >10000 | 8933 | >10000 |
| 13-2 | 9.0 | 9525 | 3280 | 2841 | 9741 | 5214 | >10000 | 2575 | >10000 |
| 14 | 1.0 | 3575 | >10000 | 2144 | >10000 | 6821 | >10000 | 1504 | >10000 |
| 14-1 | 1.9 | 9864 | 7953 | 2003 | >10000 | 7065 | >10000 | 7648 | >10000 |
| 14-2 | 13.3 | 9236 | 5894 | 6377 | >10000 | 1545 | >10000 | 1757 | >10000 |
| 15 | 15.2 | 9512 | 6345 | 8125 | 9954 | 5805 | 6659 | 9744 | 9456 |
| 16 | 17.6 | 9351 | 6023 | 8083 | 9876 | 9012 | 6740 | 9065 | 8906 |
| 17 | 21.5 | >10000 | 8546 | 7549 | 9812 | 8415 | 9047 | 8197 | 9115 |
| 18 | 15.5 | 6008 | 8045 | 7013 | 8450 | >10000 | >1000 | 8900 | 8990 |
| 19 | 15.3 | 7573 | 6548 | 9105 | 6914 | 7045 | 8451 | 9143 | 8091 |
| 20 | 18.2 | 6841 | 6357 | 5705 | >10000 | 8544 | 8253 | 9378 | 8987 |
| 21 | 6.9 | 3338 | 468 | 553 | 1808 | 1617 | 1641 | 1048 | 5871 |
| 22 | 2.3 | 5066 | 3910 | 2895 | 1832 | 5139 | 2454 | 1811 | >10000 |
| 23 | 16.7 | 8455 | 9012 | 6480 | 8405 | 7640 | 6931 | 9651 | 9091 |
| 24 | 14.5 | >10000 | >10000 | 5041 | 6085 | 4933 | 3001 | 2330 | >10000 |
| 25 | 15.9 | 8051 | 5894 | 6612 | 7603 | 6520 | 7650 | 9660 | 9753 |
| 26 | 19.0 | 5634 | 5900 | 5746 | 7334 | 6951 | 8415 | 9120 | 9412 |
| 27 | 2.3 | >1000 | >10000 | >1000 | >1000 | >1000 | >1000 | 2636 | >10000 |
| 28 | 3.0 | >10000 | >10000 | >10000 | >10000 | >10000 | >10000 | >1000 | >10000 |
| 29 | 15.0 | 7500 | 7581 | 9154 | 8045 | 7812 | 9170 | 9413 | 9003 |
| 30 | 18.1 | 7345 | 7236 | 8405 | 9450 | 8004 | 7653 | 9784 | 8760 |
| 31 | 21.3 | 8439 | 6952 | 8336 | 7946 | 7545 | 6431 | 8945 | 8707 |
| 32 | 15.2 | 8312 | 6584 | 7450 | 7891 | 9013 | 6956 | 9107 | 9451 |
| 33 | 1.8 | >10000 | >1000 | >1000 | >1000 | >1000 | >1000 | >1000 | >10000 |
| 34 | 16.8 | 9066 | 7546 | 6960 | 7031 | 9158 | 8045 | 8999 | 9354 |
| 35 | 16.4 | 7819 | 9512 | 7640 | 7716 | 9754 | 7149 | 9450 | 8884 |
| 36 | 15.1 | >10000 | 9324 | 6213 | 7164 | 6031 | 7987 | 9611 | 9000 |
| 37 | 19.8 | 5994 | 9056 | 6015 | 8045 | 7680 | 8045 | >10000 | 9378 |
| 38 | 20.4 | 6412 | 8453 | 8405 | 9144 | 9410 | 7689 | 9310 | 9238 |
| 39 | 4.7 | >1000 | >10000 | >1000 | >1000 | >1000 | >1000 | >1000 | >10000 |
| 40 | 18.2 | 7814 | 7514 | 9475 | 8512 | 7508 | 8540 | 9308 | 8980 |
| 41 | 15.5 | 7010 | 7806 | 8467 | 8095 | 6004 | 7680 | 8997 | 9111 |
| 42 | 16.1 | 6732 | 6640 | 8195 | 7601 | 6120 | 6849 | 9207 | 9413 |
| 43 | 19.3 | 9522 | 8022 | 5801 | 7885 | 6355 | 9007 | 9438 | 9465 |
| 44 | 19.0 | 7654 | 7532 | 6105 | 7688 | 7651 | >1000 | 9840 | 9271 |
| 45 | 16.4 | 9451 | >10000 | >10000 | >1000 | 8407 | 7680 | 9165 | 8506 |
| 46 | 15.8 | 7950 | 6705 | 6356 | 9110 | 7894 | 8574 | 9408 | 9569 |
| 47 | 17.4 | >1000 | 9546 | 6405 | >10000 | >10000 | 8634 | 9144 | >10000 |
| 48 | 16.0 | 8520 | 7544 | 5906 | 8665 | 7650 | 7506 | 8987 | 9008 |
| 49 | 17.5 | 9513 | 6301 | 5812 | 7532 | 8142 | >10000 | 8679 | 8779 |
| 50 | 18.4 | 7643 | >1000 | 9154 | 7472 | 7743 | 7185 | 9756 | 9044 |
| 51 | 20.7 | 7580 | 8455 | 7550 | 8744 | 6389 | 8647 | 9884 | 9458 |
| SB-271046 | 0.8 | 3498 | 313 | 4651 | 3963 | 9138 | >10000 | 4119 | >10000 |

As shown in Table 4, the compounds according to the present invention had much lower IC₅₀ levels for 5-HT6 receptor than other 5-HT receptors and dopamine receptors, and it was confirmed that the compounds had very excellent binding affinities to 5-HT6 receptor compared to other 5-HT receptors and other family receptors.

### EXPERIMENTAL EXAMPLE 3

### In vitro Functional Studies

By a method (2000) disclosed by Rutledge et al. of MDS Pharma Service (Bothell, Wash., USA, MDSPS PT#1037161), activity of adenylil cyclase in HeLa cell having transfected with human 5-HT6 receptor was measured.

Details of the assay conditions were shown in Table 5. The assay mixture consisted of Hanks' balanced salt solution(HBSS, pH 7.4) containing: 1 mM MgCl₂, 1 mM CaCl₂, 100 mM 1-methyl-3-isobutylxanthine. Incubation was started by addition of membrane suspension and compounds according to the present invention. Following the a 20 minutes incubation at 37° C., intracellular cAMP levels were measured by EIA (enzyme-immunoassay), and a compound showing inhibitory effects on serotonin(5-HT)-stimulated cAMP accumulation was classified into an antagonist. And methiothepin was used as reference 5-HT antagonist for comparison.

TABLE 5
Assay conditions of adenylyl cyclase activity in HeLa cells
transfected with human 5-HT6 receptor

| | |
|---|---|
| Target | Human HeLa cells |
| Vehicle | 0.4% DMSO |
| Incubation time/temp | 20 min at 37° C. |
| Incubation buffer | HBSS (pH. 7.4), 1 mM MgCl₂, 1 mM CaCl₂, |
| | 100 mM IBMX |
| Quantitation method | EIA quantitation of cAMP accumulation |
| Significance criteria- | ≧50% inhibition of serotonin (0.3 μM)-induced |
| Antagonist | cAMP increase |
| Significance criteria- | ≧50% increase in cAMP relative to |
| Agonist | serotonin response |

The results were shown in FIG. 1.

As shown in FIG. 1, the 5-HT concentration-dependent increase in cAMP levels with an 8.7 nM of EC₅₀, and the increase in cAMP level was inhibited by Example 13, 14 or methiothepin, a reference 5-HT6 antagonist. Particularly, Example 14 of 0.001, 0.01, 0.1, 1 and 10 μM potently inhibited the 0.3 μM serotonin (5-HT)-induced increase in cAMP levels by 10, 22, 81, 100 and 100%, respectively. And the IC₅₀ of Example 14 was 28.7 nM, which was lower than that of methiothepin(IC₅₀=60.9 nM), demonstrating significant antagonist activity. In addition, Example 14 did not show any cytotoxicity at the concentrations tested in HeLa cells transfected with the human 5-HT6 receptor.

### EXPERIMENTAL EXAMPLE 4

### In vivo Study of the Effect on Methamphetamine-Induced Disruption of Prepulse Inhibition (PPI) in Rats

To assay antipsychotic properties of the compounds according to the invention, prepulse inhibition (PPI) of acoustic startle in animals was performed.

Startle response was measured using SR-LAB startle chamber (San Diego Instruments, San Diego, USA).

The animal enclosure was housed in a ventilated and sound-attenuated startle chamber with 60 dB ambient noise level, and consisted of a Plexiglas cylinder 40 mm in diameter on a platform, connected to a piezoelectric accelerometer which detects and transducer motion within the cylinder. Acoustic noise bursts were presented through a loudspeaker mounted 24 [symbol] above the animal.

Behavioral testing was performed between 10 a.m. and 5 p.m., during the light phase by a modified Mansbach et al's method [Mansbach R S, Brooks E W, Sanner M A, Zorn S H, Selective dopamine D4 receptor antagonists reverse apomorphine-induced blockade of prepulse inhibition., Psychopharmacology(Berl), 135:194-200, 1998]. Each startle session began with a 5-min acclimatization period in the chamber to 68 dB background noises. The test session consisting of the following four different trial types was carried for all experiments: a 40 ms broadband 120 dB burst (P; pulse alone trial), P preceded 100 ms earlier by a 20 ms noise burst 10 dB above background (pP; prepulse+pulse trial), a 40 ms broadband 78 dB burst (prepulse alone trial), and a no stimulus trial (background). Eight trials of each type were presented in a pseudorandom order (total32 trials) with an average interval of 15 sec. separating each trial. An extra 5 pulse-alone trials were presented at the beginning and end of each test session, but were not used in the calculation of PPI values. PPI was defined as the percent reduction in startle amplitude in the presence of prepulse compared to the amplitude in the absence of the prepulse using the following Math Equation 1.

PPI (%)=[100−(100×startle amplitude on pP trial/startle amplitude on P trial)] <Math Equation 1>

The rats were administered (i.p.) with the compounds according to the invention(25 or 50 [symbol] , SB-271046(positive control, 50 [symbol] ) or vehicle, 30 min before the injection of methamphetamine (3 [symbol] i.p.), and were placed in the startle chamber 30 min after the methamphetamine injection for testing. The compounds according to the invention or SB-271046 were suspended in 3% Tween 80 solution.

Statistical significance of the results was evaluated by one-way analysis of variance (ANOVA) with Dunnett's post-hoc tests for comparing control to treatment. Differences were considered significant at P<0.05. Statistical analyses were conducted using SigmaStat software (SigmaStat, Jandel Co., San Rafael, Calif.). The data were expressed as means±SEM.

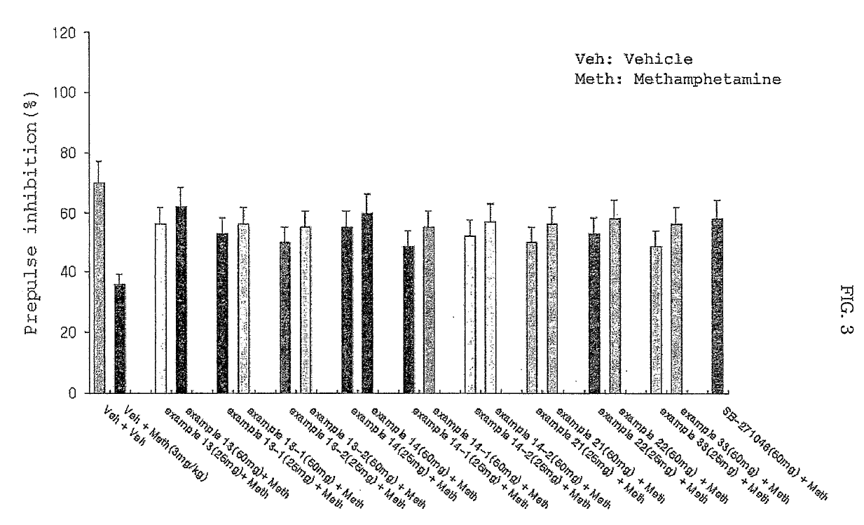

The results were shown in FIGS. 2 and 3.

As shown in FIG. 2 and 3, the compounds according to the invention(25 or 50 [symbol] i.p.) alone had no significant effect on PPI when compared to vehicle (negative control) in rats. However, the disruption of PPI by methamphetamine (3 [symbol] i.p.) was reversed significantly by pretreatment with the compounds according to the invention (P<0.05) and SB-271046 (P<0.05), indicating significant antipsychotic activity. Also, there were no significant differences in mean startle amplitude of the compounds according to the invention including Example 13 and 14, or SB-271046 administered 30 min before methamphetamine when compared with that of methamphetamine group.

### EXPERIMENTAL EXAMPLE 5

### Effect on Rotarod Deficit in Mice

The mouse was placed on a 1 inch diameter knurled plastic rod rotating at 6 rpm (Ugo-Basile, Milano, Italy), and the rotarod deficit (%) was obtained by counting the number of animals fallen from the rotating rod within 1 min [Dunham et al., 1957] at 60, 90 and 120 min after the injection of the compound according to the invention(200, 300 or 400 [symbol] ). The median neurotoxic dose (TD₅₀) was determined as the dose at which 50% of animals showed rotarod deficit. The compounds of the examples were suspended in 3% Tween 80 solution, and were administered (p.o.) 60 min before the testing.

The result was shown in Table 6.

TABLE 6
Effect on rotarod deficit in mice of the compounds
according to the present invention

| | Rotarod deficit | | | |
|---|---|---|---|---|
| | (%)( [symbol] p.o.) | | | |
| Example | 200 | 300 | 400 | TD₅₀ ( [symbol] p.o.) |
| 1 | 0 | 0 | 0 | >>400 |
| 2 | 0 | 0 | 0 | >>400 |
| 3 | 0 | 0 | 0 | >>400 |
| 6 | 0 | 0 | 0 | >>400 |
| 9 | 0 | 0 | 0 | >>400 |
| 10 | 0 | 0 | 0 | >>400 |
| 11 | 0 | 0 | 0 | >>400 |
| 12 | 0 | 0 | 0 | >>400 |
| 13 | 0 | 0 | 0 | >>400 |
| 13-1 | 0 | 0 | 0 | >>400 |
| 13-2 | 0 | 0 | 0 | >>400 |
| 14 | 0 | 0 | 0 | >>400 |
| 14-1 | 0 | 0 | 0 | >>400 |
| 14-2 | 0 | 0 | 0 | >>400 |
| 21 | 0 | 0 | 0 | >>400 |
| 22 | 0 | 0 | 0 | >>400 |
| 24 | 0 | 0 | 0 | >>400 |
| 27 | 0 | 0 | 0 | >>400 |
| 28 | 0 | 0 | 0 | >>400 |
| 33 | 0 | 0 | 0 | >>400 |
| 39 | 0 | 0 | 0 | >>400 |

As shown in Table 6, a single administration (p.o.) of compounds according to the invention did not show any rotarod ataxia at the doses up to 400 [symbol] for 120 min after the treatment. Thus, their median neurotoxic dose (TD₅₀) was calculated to more than 400 [symbol] (p.o.) each in mice, demonstrating that compounds according to the invention have much lower liability to induce extrapyramidal side effects.

### FORMULATION EXAMPLE 1

### Pharmaceutical Formulations

1-1. Preparation of Powder

The compound according to the present invention, a pharmaceutically acceptable salt or a rodrug thereof 2 g

Lactose 1 g

Powder product was prepared by mixing the above ingredients and filling an airtight package therewith.

1-2. Preparation of Tablet

The compound according to the present invention, a pharmaceutically acceptable salt or a rodrug thereof 100

[symbol]

Corn starch 100

[symbol]

Lactose 100

[symbol]

Magnesium stearate 2

[symbol]

Tablets were prepared by mixing the above ingredients and tabletting by a conventional method.

1-3. Preparation of Capsule

The compound according to the present invention, a pharmaceutically acceptable salt or a rodrug thereof 100

[symbol]

Corn starch 100

[symbol]

Lactose 100

[symbol]

Magnesium stearate 2

[symbol]

Capsules were prepared by mixing the above ingredients and filling a gelatin capsule by a conventional method.

### INDUSTRIAL APPLICABILITY

The compounds of N-substituted-1H-quinoline-2,4-diones according to the present invention have excellent binding affinity to the 5HT6 receptor, excellent selectivity for the 5HT6 receptor over other receptors, the inhibitory effect of the serotonin(5-HT)-stimulated cAMP accumulation and an effect on methamphetamine(2 [symbol] i.p.)-induced disruption of prepulse inhibition (PPI) in rats. Also, the compounds of the present invention below 400 [symbol] don't show any rotarod deficits in mice. Therefore, The compounds of N-substituted-1H-quinoline-2,4-diones according to the present invention may be useful to composition for treatment of a 5HT6 receptor relating disorders such as cognitive disorders, Alzheimers disease, anxiety, depression, schizophrenia, stress disorder, panic disorder, phobic disorder, obsessive compulsive disorder, post traumatic stress disorder, immune system depression, psychosis, paraphrenia, mania, convulsive disorder, personality disorder, migraine, drug addiction, alcoholism, obesity, eating disorder, and sleep disorder.

1. A compound of N-substituted-1H-quinoline-2,4-dione represented by the following formula 1 or a pharmaceutically acceptable salt thereof. 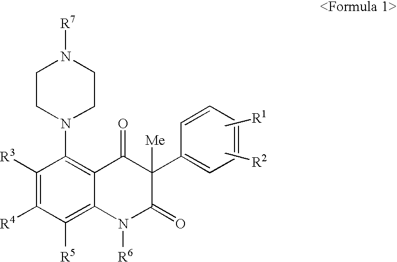
wherein,
R¹ and R² independently represent a hydrogen, halogen, nitro, amino, amino substituted by one or two alkyl, cyclic amino, carboxylic acid, thiol, cyano, alkyl, aryl, heteroaryl, alkoxy, aryloxy, acyloxy, acylamino, arylsulfonylamino, arylsulfonylureido, alkylthio, arylthio, alkylcarboxylate, arylcarboxylate, aralkylcarboxylate, alkylureido, arylureido, alkylamidino or arylamidino;
R³, R⁴ and R⁵ independently represent a hydrogen, halogen, amino, cyclic amino, nitro, cyano, alkyl, haloalkyl, alkoxy, haloalkoxy, piperidinyl, or N-methyl piperidinyl;
R⁶ represents alkyl, aryl, cycloalkyl, arylalkyl, heteroaryl or heteroarylalkyl; and
R⁷ represents hydrogen, alkyl or aryl.

2. The compound of N-substituted-1H-quinoline-2,4-dione or a pharmaceutically acceptable salt thereof according to claim 1, wherein
R¹ and R² are independently a hydrogen, halogen, C₁˜C₄ alkoxy, amino, amino substituted by one or two C₁˜C₄ alkyl, nitro or benzyloxy;
R³, R⁴ and R⁵ are independently a hydrogen, halogen or C₁˜C₄ alkoxy;
R⁶ represents a C₁˜C₄ alkyl; C₃˜C₇ cycloalkyl C₁˜C₂ alkyl; benzyl substituted by a substituent selected from the group consisting of hydrogen, nitro, amino, halogen and C₁˜C₄ alkoxyphenyl; naphthalenylmethyl; or heteroaryl C₁˜C₂ alkyl substituted by a substituent selected from a the group consisting of pyridine, quinoline and benzoimidazole; and
R⁷ is a hydrogen or C₁˜C₄ alkyl.

3. The compound of N-substituted-1H-quinoline-2,4-dione or a pharmaceutically acceptable salt thereof according to claim 2, wherein
R¹ is a hydrogen, fluorine, chlorine, bromine, iodine, methoxy, ethoxy, amino, methylamino, ethylamino, dimethylamino, diethylamino, nitro or benzyloxy;
R² is a hydrogen, fluorine, chlorine, bromine, iodine, methoxy, nitro, amino or benzyloxy;
R³, R⁴ and R⁵ are independently a hydrogen, chlorine, bromine or methoxy;
R⁶ represents a methyl, ethyl, cyclohexylmethyl, benzyl, nitrobenzyl, aminobenzyl, methoxybenzyl, bromobenzyl, biphenylmethyl, naphthalenylmethyl, pyridinylmethyl, quinolinylmethyl or benzoimidazolylmethyl; and
R⁷ is a hydrogen, methyl or ethyl.

4. The compound of N-substituted-1H-quinoline-2,4-dione or a pharmaceutically acceptable salt thereof according to claim 1, selected from the group consisting of:
1-Benzyl-7-chloro-3-(4-methoxy-phenyl)-3-methyl-5-(4-methyl-piperazin-1-yl)-1H-quinoline-2,4-dione;
1-Benzyl-7-chloro-3-(4-hydroxy-phenyl)-3-methyl-5-(4-methyl-piperazin-1-yl)-1H-quinoline-2,4-dione;
1-Benzyl-7-chloro-3-methyl-5-(4-methyl-piperazin-1-yl)-3-(4-nitro-phenyl)-1H-quinoline-2,4-dione;
3-(4-Amino-phenyl)-1-benzyl-7-chloro-3-methyl-5-(4-methyl-piperazin-1-yl)-1H-quinoline-2,4-dione;
1-Benzyl-7-chloro-3-(4-diethylamino-phenyl)-3-methyl-5-(4-methyl-piperazin-1-yl)-1H-quinoline-2,4-dione;
1-Benzyl-7-chloro-3-(4-ethylamino-phenyl)-3-methyl-5-(4-methyl-piperazin-1-yl)-1H-quinoline-2,4-dione;
7-Chloro-3-(4-methoxy-phenyl)-3-methyl-5-(4-methyl-piperazin-1-yl)-1-(3-nitro-benzyl)-1H-quinioline-2,4-dione;
7-Chloro-3-(4-hydroxy-phenyl)-3-methyl-5-(4-methyl-piperazin-1-yl)-1-(3-nitro-benzyl)-1H-quinoline-2,4-dione;
1-(3-Amino-benzyl)-7-chloro-3-(4-methoxy-phenyl)-3-methyl-5-(4-methyl-piperazin-1-yl)-1H-quinoline-2,4-dione;
1-(3-Amino-benzyl)-7-chloro-3-(4-hydroxy-phenyl)-3-methyl-5-(4-methyl-piperazin-1-yl)-1H-quinoline-2,4-dione;
1-Benzyl-7-chloro-3-methyl-5-(4-methyl-piperazin-1-yl)-3-phenyl-1H-quinoline-2,4-dione;
1-Benzyl-3-(4-benzyloxy-3-bromo-phenyl)-7-chloro-3-methyl-5-(4-methyl-piperazin-1-yl)-1H-quinoline-2,4-dione
1-Benzyl -7-chloro-3-(4-methoxy-phenyl)-3-methyl-5-piperazin-1-yl-1H-quinoline-2,4-dione;
(S)-1-Benzyl -7-chloro-3-(4-methoxy-phenyl)-3-methyl-5-piperazin-1-yl-1H-quinoline-2,4-dione;
(R)-1-Benzyl-7-chloro-3-(4-methoxy-phenyl)-3-methyl-5-piperazin-1-yl-1H-quinoline-2,4-dione;
1-Benzyl-7-chloro-3-(4-hydroxy-phenyl)-3-methyl-5-piperazin-1-yl-1H-quinoline-2,4-dione;
(S)-1-Benzyl-7-chloro-3-(4-hydroxy-phenyl)-3-methyl-5-piperazin-1-yl-1H-quinoline-2,4-dione;
(R)-1-Benzyl-7-chloro-3-(4-hydroxy-phenyl)-3-methyl-5-piperazin-1-yl-1H-quinoline-2,4-dione;
1-Benzyl-7-chloro-3-methyl-3-(4-nitro-phenyl)-5-piperazin-1-yl-1H-quinoline-2,4-dione;
3-(4-Amino-phenyl)-1-benzyl-7-chloro-3-methyl-5-piperazin-1-yl-1H-quinoline-2,4-dione;
1-Benzyl-7-chloro-3-(4-diethylamino-phenyl)-3-methyl-5-piperazin-1-yl-1H-quinoline-2,4-dione;
1-Benzyl-7-chloro-3-(4-ethylamino-phenyl)-3-methyl-5-piperazin-1-yl-1H-quinoline-2,4-dione;
1-Benzyl-7-chloro-3-(4-chloro-phenyl)-3-methyl-5-piperazin-1-yl-1H-quinoline-2,4-dione;
1-Benzyl-3-(4-bromo-phenyl)-7-chloro-3-methyl-5-piperazin-1-yl-1H-quinoline-2,4-dione;
1-Benzyl-7-chloro-3-(4-iodo-phenyl)-3-methyl-5-piperazin-1-yl-1H-quinoline-2,4-dione;
1-Benzyl-7-chloro-3-methyl-5-phenyl-5-piperazin-1-yl-1H-quinoline-2,4-dione;
7-Chloro-3-(4-methoxy-phenyl)-3-methyl-1-(3-nitro-benzyl)-5-piperazin-1-yl-1H-quinoline-2,4-dione;
7-Chloro-3-(4-hydroxy-phenyl)-3-methyl-1-(3-nitro-benzyl)-5-piperazin-1-yl-1H-quinoline-2,4-dione;
1-(3-Amino-benzyl)-7-chloro-3-(4-methoxy-phenyl)-3-methyl-5-piperazin-1-yl-1H-quinoline-2,4-dione;
1-(3-Amino-benzyl)-7-chloro-3-(4-hydroxy-phenyl)-3-methyl-5-piperazin-1-yl-1H-quinoline-2,4-dione;
7-Chloro-1-(3-methoxy-benzyl)-3-(4-methoxy-phenyl)-3-methyl-5-piperazin-1-yl-1H-quinoline-2,4-dione;
7-Chloro-1-(3-hydroxy-benzyl)-3-(4-hydroxy-phenyl)-3-methyl-5-piperazin-1-yl-1H-quinoline-2,4-dione;
7-Chloro-1-(2-methoxy-benzyl)-3-(4-methoxy-phenyl)-3-methyl-5-piperazin-1-yl-1H-quinoline-2,4-dione;
7-Chloro-1-(2-hydroxy-benzyl)-3-(4-hydroxy-phenyl)-3-methyl-5-piperazin-1-yl-1H-quinoline-2,4-dione;
7-Chloro-1-(4-methoxy-benzyl)-3-(4-methoxy-phenyl)-3-methyl-5-piperazin-1-yl-1H-quinoline-2,4-dione;
7-Chloro-1-(4-hydroxy-benzyl)-3-(4-hydroxy-phenyl)-3-methyl-5-piperazin-1-yl-1H-quinoline-2,4-dione;
1-(3-Bromo-benzyl)-7-chloro-3-(4-methoxy-phenyl)-3-methyl-5-piperazin-1-yl-1H-quinoline-2,4-dione;
1-(3-Bromo-benzyl)-7-chloro-3-(4-hydroxy-phenyl)-3-methyl-5-piperazin-1-yl-1H-quinoline-2,4-dione;
1-(2-Bromo-benzyl)-7-chloro-3-(4-methoxy-phenyl)-3-methyl-5-piperazin-1-yl-1H-quinoline-2,4-dione;
1-(2-Bromo-benzyl)-7-chloro-3-(4-hydroxy-phenyl)-3-methyl-5-piperazin-1-yl-1H-quinoline-2,4-dione;
7-Chloro-3-(4-methoxy-phenyl) -3-methyl-5-piperazin-1-yl-1-pyridin-3-ylmethyl-1H-quinoline-2,4-dione;
7-Chloro-3-(4-hydroxy-phenyl)-3-methyl-5-piperazin-1-yl-1-pyridin-3-ylmethyl-1H-quinoline-2,4-dione;
7-Chloro-3-(4-methoxy-phenyl)-3-methyl-1-naphthalen-2-ylmethyl-5-piperazin-1-yl-1H-quinoline-2,4-dione;
7-Chloro-3-(4-hydroxy-phenyl)-3-methyl-1-naphthalen-2-ylmethyl-5-piperazin-1-yl-1H-quinoline-2,4-dione;
1-Biphenyl-4-ylmethyl-7-chloro-3-(4-methoxy-phenyl)-3-methyl-5-piperazin-1-yl-1H-quinoline-2,4-dione;
1-Biphenyl-4-ylmethyl-7-chloro-3-(4-hydroxy-phenyl)-3-methyl-5-piperazin-1-yl-1H-quinoline-2,4-dione;
1-(1H-Benzoimidazol-2-ylmethyl)-7-chloro-3-(4-methoxy-phenyl)-3-methyl-3-piperazin-1-yl-1H-quinoline-2,4-dione;
7-Chloro-3-(4-methoxy-phenyl)-3-methyl-5-piperazin-1-yl-1-quinolin-2-ylmethyl-1H-quinoline-2,4-dione;
7-Chloro-3-(4-hydroxy-phenyl)-3-methyl-5-piperazin-1-yl-1-quinolin-2-ylmethyl-1H-quinoline-2,4-dione;
7-Chloro-1-ethyl-3-(4-methloxy-phenyl)-3-methyl-5-piperazin-1-yl-1H-quinoline-2,4-dione;
5-Chloro-1-ethyl-3-(4-hydroxy-phenyl)-3-methyl-7-piperazin-1-yl-1H-quinoline-2,4-dione;
7-Chloro-1-cyclohexylmethyl-3-(4-methoxy-phenyl)-3-methyl-5-piperazin-1-yl-1H-quinoline-2,4-dione;
7-Chloro-1-cyclohexylmethyl-3-(4-hydroxy-phenyl)-3-methyl-5-piperazin-1-yl-1H-quinoline-2,4-dione;
1-Benzyl-7-chloro-3-(3-methoxy-phenyl)-3-methyl-5-piperazin-1-yl-1H-quinoline-2,4-dione; and
1-Benzyl-7-chloro-3-(3-hydroxy-phenyl)-3-methyl-5-piperazin-1-yl-1H-quinoline-2,4-dione.

5. A process of preparing the compound of N-substituted-1H-quinoline-2,4-diones of claim 1 as represented in scheme 1, comprising:
(a) preparing an intermediate I by a coupling reaction of compound 2 and compound 3;
(b) preparing an intermediate II by a cyclization reaction of the intermediate I in the presence of a base;
(c) preparing an intermediate III by a substitution reaction on N(1) of the intermediate II in the presence of a an electrophilic group and a base; and
(d) preparing a compound of Formula 1 by substituting the intermediate III with an amine. 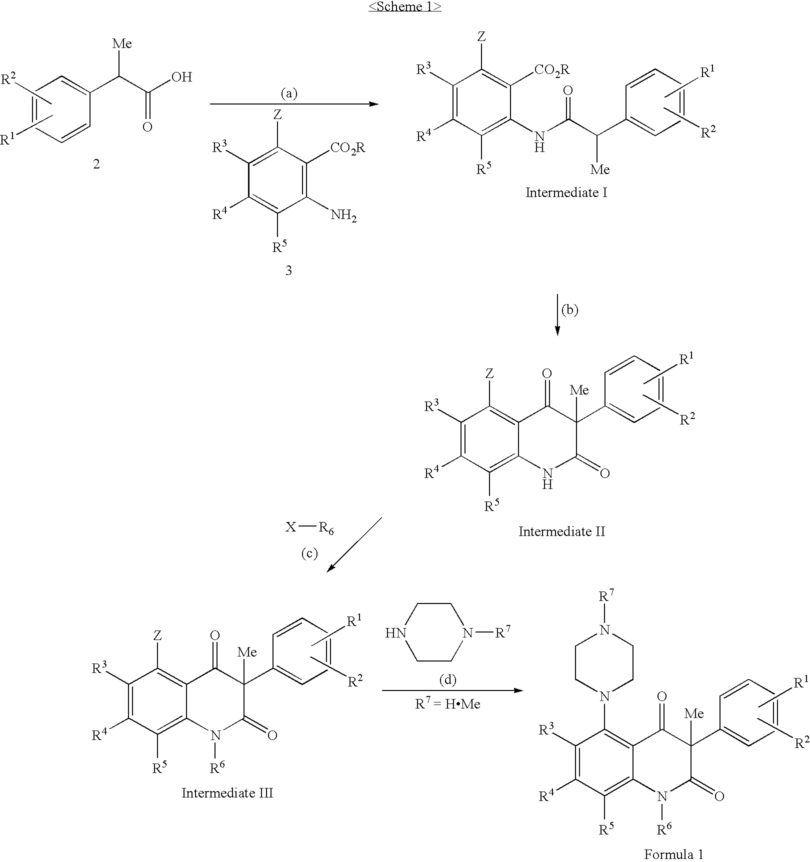
wherein
R¹˜R⁷ are the same as defined in Formula 1 of claim 1;
R is a methyl, ethyl, or propyl group, and
Z represents a halogen as selected from the group consisting of fluorine, chlorine, bromine and iodine, and
X is chlorine, bromine, iodine, o-methlylsulfonyl or o-toluenesulfonyl.)

6. The process according to claim 5, wherein the R¹-, R²- or R⁶-substituents of Formula 1 is further transformed into hydroxy(OH) under the presence of a boron tribromide when the R¹-, R²- or R⁶-substituents are methoxy.

7. The process according to claim 5, wherein the R¹-, R²- or R⁶-substituents of Formula 1 is further transformed into amino under the presence of a tin(II) dihydrate when the R¹-, R²- or R⁶-substituents are nitro(NO₂).

8. The process according to claim 7, wherein the amino is further transformed into mono- or di-alkylamino under the presence of a sodium cyanoborohydride, and formaldehyde or acetaldehyde.

9. The process according to claim 5, wherein the coupling reaction comprises:
(1) forming an acid chloride by reacting compound 2 with chlorinating agent selected from the group consisting of SOCl₂, (COCl)₂, PCl₅, and BOP-Cl (bis(2-oxo-diazolindinyl)phosphinic chloride) in an inert solvent; and
(2) coupling the acid chloride of compound 2 and compound 3 in an inert solvent by mixing and heating them.

10. The process according to claim 5, wherein the amine is N-methylpiperazine or piperazine.

11. A pharmaceutical composition for a 5-HT6 serotonin receptor antagonist containing the compound of claim 1, a pharmaceutically acceptable salt thereof or a prodrug thereof as an active ingredient.

12. A pharmaceutical composition for treatment of central nervous system disorders containing the compound of claim 1, a pharmaceutically acceptable salt thereof or a prodrug thereof as an active ingredient.

13. The pharmaceutical composition of claim 12, wherein the disorders of the central nervous system are cognitive disorders, Alzheimers disease, anxiety, depression, schizophrenia, stress disorder, panic disorder, phobic disorder, obsessive compulsive disorder, post traumatic stress disorder, immune system depression, psychosis, paraphrenia, mania, convulsive disorder, personality disorder, migraine, drug addiction, alcoholism, obesity, eating disorder, or sleep disorder.

14. A pharmaceutical composition for a 5-HT6 serotonin receptor antagonist containing the compound of claim 2, a pharmaceutically acceptable salt thereof, or a prodrug thereof as an active ingredient.

15. A pharmaceutical composition for a 5-HT6 scrotonin receptor antagonist containing the compound of claim 4, a pharmaceutically acceptable salt thereof, or a prodrug thereof as an active ingredient.

16. A pharmaceutical composition for treatment of central nervous system disorders containing the compound of claim 2, a pharmaceutically acceptable salt thereof, or a prodrug thereof as an active ingredient.

17. A pharmaceutical composition for treatment of central nervous system disorders containing the compound of claim 3, a pharmaceutically acceptable salt thereof or a prodrug thereof as an active ingredient.

18. A pharmaceutical composition for treatment of central nervous system disorders containing the compound of claim 4, a pharmaceutically acceptable salt thereof, or a prodrug thereof as an active ingredient.

19. A method for treating central nervous system disorders in a mammal comprising:
administering an effective amount of the compound of claim 1, a pharmaceutically acceptable salt thereof or a prodrug thereof, to a mammal in need thereof.

20. The method of claim 19, wherein the disorders of the central nervous system are cognitive disorders, Alzheimers disease, anxiety, depression, schizophrenia, stress disorder, panic disorder, phobic disorder, obsessive compulsive disorder, post traumatic stress disorder, immune system depression, psychosis, paraphrenia, mania, convulsive disorder, personality disorder, migraine, drug addiction, alcoholism, obesity, eating disorder, or sleep disorder.
